SEQUENCE LISTING (1 sequence)

SEQ ID NO: 1 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

(19) United States
(12) Patent Application Publication
(10) Pub. No.: US 2016/0074374 A1
(43) Pub. Date: Mar. 17, 2016

(54) CORRECTORS ACTING THROUGH MSD1 OF CFTR PROTEIN

(71) Applicants: VERTEX PHARMACEUTICALS INCORPORATED, Boston, MA (US); THE UNIVERSITY OF NORTH CAROLINA AT CHAPEL HILL, Chapel Hill, NC (US)

(72) Inventors: Fredrick F. Van Goor, San Diego, CA (US); Beth Jennifer Hoffman, San Diego, CA (US); Oxana Adolfovna de la Rosa, San Diego, CA (US); Douglas Cyr, Chapel Hill, NC (US)

(21) Appl. No.: 14/787,061

(22) PCT Filed: Apr. 25, 2014
(86) PCT No.: PCT/US2014/035546

§ 371 Date: Oct. 26, 2015

Related U.S. Application Data
(60) Provisional application No. 61/821,607, filed on May 9, 2013.
(60) Provisional application No. 61/821,611, filed on May 9, 2013.
(60) Provisional application No. 61/816,630, filed on Apr. 26, 2013.
(60) Provisional application No. 61/816,635, filed on Apr. 26, 2013.

(51) Int. Cl. A61K 31/443; C07K 16/28; G01N 33/68; A61K 39/395; A61K 45/06; A61K 38/05; A61K 38/02; A61K 31/47

(52) U.S. Cl. CPC A61K 31/443; A61K 38/02; C07K 16/28; A61K 31/47; A61K 39/3955; A61K 45/06; A61K 38/05; G01N 33/6872; G01N 2500/10; G01N 2800/7052

(57) ABSTRACT

The present disclosure provides methods for treating Cystic Fibrosis in a subject by administering to the subject a corrector agent capable of acting through MSD1 during the biosynthesis of CFTR protein. The disclosure also provides methods of screening for new corrector agents capable of acting through MSD1 during the biosynthesis of a CFTR protein.

### RELATED APPLICATIONS

This application claims the benefit of priority to U.S. provisional applications 61/816,630, filed on Apr. 26, 2013; 61/816,635, filed on Apr. 26, 2013; 61/821,607, filed on May 9, 2013; and 61/821,611, filed on May 9, 2013, which are hereby incorporated herein by reference in their entirety.

### FUNDING

This invention was made with government support under Grant Nos. GM056981 and GM067785 awarded by the National Institutes of Health. The government has certain rights in the invention.

### BACKGROUND OF THE INVENTION

Cystic Fibrosis (CF) is a fatal autosomal recessive disease associated with defective hydration of lung airways due to the loss of function of the CF transmembrane conductance regulator (CFTR) channel at epithelial cell surfaces. CFTR is a 1480 amino acid ABC-transporter protein. It contains 12 transmembrane spanning segments (TM), which are organized in the primary structure into two membrane spanning domains (membrane spanning domain 1 (MSD1) and MSD2), two cytosolic nucleotide-binding domains (NBD1 and NBD2), and a regulatory domain (R) (Riordan et al., 2008, Annu Rev Biochem, 77: 701-26). MSD1 contains transmembrane spanning segments 1 to 6 (TM1-6) and MSD2 contains transmembrane spanning segments 7 to 12 (TM7-12). The TM segments of CFTR assemble into a complex with the NBDs to form an ATP-gated anion channel (Riordan et al., 1989, Science, 245: 1066-73; Riordan et al., 2008, Annu Rev Biochem, 77: 701-26; Anderson, et al., 1991, Science, 253: 202-5).

CFTR loss of function in humans suffering from CF is frequently caused by mutations in the CFTR gene that cause misfolding and premature degradation of the mutant CFTR protein. These mutations result in a loss of functional CFTR protein at the cell surface (Rowe et al., 2005, N Engl J Med, 352: 1992-2001; Denning et al., 1992, Nature, 358: 761-4; Riordan et al., 1989, Science, 245: 1066-73; Cheng, 1990, Cell, 63:827-34). One such mutation is a deletion of phenylalanine at amino acid residue 508 (ΔF508) from NBD1 of human CFTR. In the absence of F508, folding of CFTR's domains initiates, but channel assembly is arrested at an intermediate stage (Rosser et al., 2008, Mol Biol Cell, 19: 4570-79; Younger et al., 2006, Cell, 126:571-82; Serohijos, et al., 2008, PNAS, 105, 3256-61; Lukacs et al., 1994, EMBO Journal, 13: 6076-86).

ΔF508-CFTR function is partially restored in bronchial epithelial cells from CF subjects by lumacaftor (also known as VX-809 or 3-{6-{[1-(2,2-difluoro-1,3-benzodioxol-5-yl)cyclopropanecarbonyl]amino}-3-methylpyridin-2-yl}benzoic acid) (Van Goor et al., 2011, PNAS, 108: 18843-48) and Corr-4a (Rosser et al., 2008, Mol Biol Cell, 19: 4570-79). In primary cultures of human bronchial epithelial cells isolated from subjects with CF who are homozygous for ΔF508, lumacaftor increased chloride transport from a baseline of 3% to 14% of normal (Van Goor et al., 2011, PNAS, 108: 18843-48). In a 28-day clinical study of CF subjects homozygous for ΔF508-CFTR, lumacaftor (200 mg qd) also improved CFTR function as determined by a drop in the sweat chloride concentration (Clancy, et al., 2012, Thorax, 67:12-18). Although lumacaftor and Corr-4a exhibit some effect on ΔF508-CFTR, they nevertheless only partially restore ΔF508-CFTR function. Therefore, agents that increase CFTR activity further are likely to be necessary in CF therapy.

### SUMMARY OF THE DISCLOSURE

The present invention is based on the surprising discovery that a corrector agent may be designed and identified to act through the membrane spanning domain 1 (MSD1) of a CFTR protein having a mutation in the NBD1 domain, i.e., ΔF508, in order to improve mutant CFTR function in the treatment of CF.

In one aspect, the invention relates to a method of treating cystic fibrosis in a patient, comprising the step of: administering to the patient a corrector agent capable of acting through the membrane spanning domain 1 (MSD1) during the biosynthesis of a wildtype or mutant CFTR protein, provided that the corrector agent is not a compound listed in Table 1, wherein the action is characterized in vitro by one or more of the following: (i) an increase in accumulation of fragment CFTR³⁷⁵ in a cell expressing the fragment in the presence of the corrector compared to such accumulation of fragment CFTR³⁷⁵ in a cell expressing the fragment in the absence of the corrector, (ii) an increase in accumulation of fragment CFTR³⁸⁰ in a cell expressing the fragment in the presence of the corrector compared to such accumulation of fragment CFTR³⁸⁰ in a cell expressing the fragment in the absence of the corrector, (iii) an increase in the half-life of fragment CFTR³⁷⁵ in a cell expressing the fragment in the presence of the corrector compared to such half-life of fragment CFTR³⁷⁵ in a cell expressing the fragment in the absence of the corrector, (iv) an increase in the half-life of fragment CFTR³⁸⁰ in a cell expressing the fragment in the presence of the corrector compared to such half-life of fragment CFTR³⁸⁰ in a cell expressing the fragment in the absence of the corrector, (v) an increase in the half-life of fragment CFTR³⁸⁰, CFTR⁴³⁰, and/or CFTR⁶⁵³ in a cell expressing CFTR³⁸⁰, CFTR⁴³⁰, and/or CFTR⁶⁵³ in the presence of said corrector compared to the half-life of CFTR³⁸⁰, CFTR⁴³⁰, and/or CFTR⁶⁵³, respectively, in a cell expressing said fragment in the absence of said corrector, or (vi) an enhanced resistance of fragment CFTR³⁸⁰ to proteolysis with trypsin in the presence of the corrector compared to such proteolysis in the absence of the corrector. In some embodiments, the corrector agent action is characterized by one, two, three, four, five, or six characteristics selected from characteristics (i)-(vi). In some embodiments, the concentration of said corrector agent needed to achieve the maximal accumulation of fragment CFTR³⁸⁰ in a cell expressing said fragment is about the same concentration of said corrector agent needed to achieve the maximal accumulation of full-length CFTR in a cell expressing said full-length CFTR.

In some embodiments, this invention relates to corrector agents, as defined above, to pharmaceutical compositions containing those corrector agents, and to methods of using those corrector agents or compositions.

In one aspect, the invention relates to a pharmaceutical composition comprising a corrector agent capable of acting through the membrane spanning domain 1 (MSD1) during the biosynthesis of a wildtype or mutant CFTR protein, provided that the corrector agent is not a compound listed in Table 1, wherein the action is characterized in vitro by one or more of the following: (i) an increase in accumulation of fragment CFTR³⁷⁵ in a cell expressing the fragment in the presence of the corrector compared to such accumulation of fragment CFTR³⁷⁵ in a cell expressing the fragment in the absence of the corrector, (ii) an increase in accumulation of fragment CFTR³⁸⁰ in a cell expressing the fragment in the presence of the corrector compared to such accumulation of fragment CFTR³⁸⁰ in a cell expressing the fragment in the absence of the corrector, (iii) an increase in the half-life of fragment CFTR³⁷⁵ in a cell expressing the fragment in the presence of the corrector compared to such half-life of fragment CFTR³⁷⁵ in a cell expressing the fragment in the absence of the corrector, (iv) an increase in the half-life of fragment CFTR³⁸⁰ in a cell expressing the fragment in the presence of the corrector compared to such half-life of fragment CFTR³⁸⁰ in a cell expressing the fragment in the absence of the corrector, (v) an increase in the half-life of fragment CFTR³⁸⁰, CFTR⁴³⁰, and/or CFTR⁶⁵³ in a cell expressing CFTR³⁸⁰, CFTR⁴³⁰, and/or CFTR⁶⁵³ in the presence of said corrector compared to the half-life of CFTR³⁸⁰, CFTR⁴³⁰, and/or CFTR⁶⁵³, respectively, in a cell expressing said fragment in the absence of said corrector, or (vi) an enhanced resistance of fragment CFTR³⁸⁰ to proteolysis with trypsin in the presence of the corrector compared to such proteolysis in the absence of the corrector, and a pharmaceutically acceptable acceptable carrier, adjuvant or vehicle. In some embodiments, the corrector agent action is characterized by one, two, three, four, five, six or seven characteristics selected from characteristics (i)-(vi). In some embodiments, the concentration of said corrector agent needed to achieve the maximal accumulation of fragment CFTR³⁸⁰ in a cell expressing said fragment is about the same concentration of said corrector agent needed to achieve the maximal accumulation of full-length CFTR in a cell expressing said full-length CFTR.

In some embodiments, the increases in half-life values for fragments CFTR³⁸⁰, CFTR⁴³⁰, and/or CFTR⁶⁵³ in a cell expressing said fragment CFTR³⁸⁰, CFTR⁴³⁰, and/or CFTR⁶⁵³ in the presence of said corrector are comparable to the increases in half-life values for fragments CFTR³⁸⁰, CFTR⁴³⁰, and/or CFTR⁶⁵³ in a cell expressing said fragment CFTR³⁸⁰, CFTR⁴³⁰, and/or CFTR⁶⁵³ in the absence of said corrector,

In some embodiments, the corrector agent used in the methods and compositions of the invention acts through at least one amino acid residue selected from an amino acid residue corresponding to amino acid residues 362-380 of CFTR (SEQ ID NO: 1). In some embodiments, the corrector agent used in the methods and compositions of the invention acts through at least one amino acid residue selected from an amino acid residue corresponding to amino acid residues 371-375 of CFTR (SEQ ID NO: 1).

In some embodiments, the corrector agent used in the methods and compositions of the invention is characterized in vitro by an at least 2-fold, at least 4-fold or at least 6-fold increase in accumulation of fragment CFTR³⁷⁵ in a cell expressing the fragment in the presence of the corrector compared to such accumulation of fragment CFTR³⁷⁵ in a cell expressing the fragment in the absence of the corrector. In some embodiments, the corrector agent used in the methods and compositions of the invention is characterized in vitro by an at least 2-fold, at least 4-fold or at least 6-fold increase in accumulation of fragment CFTR³⁸⁰ in a cell expressing the fragment the presence of the corrector compared to such accumulation of fragment CFTR³⁸⁰ in a cell expressing the fragment in the absence of the corrector.

In some embodiments, the corrector agent used in the methods and compositions of the invention is characterized in vitro by an at least 2-fold, at least 4-fold or at least 6-fold increase in the half-life of fragment CFTR³⁷⁵ in a cell expressing the fragment in the presence of the corrector compared to such half-life of fragment CFTR³⁷⁵ in a cell expressing the fragment in the absence of the corrector. In some embodiments, the corrector agent used in the methods and compositions of the invention is characterized in vitro by an at least 2-fold, at least 4-fold or at least 6-fold increase in the half-life of fragment CFTR³⁸⁰ in a cell expressing the fragment in the presence of the corrector compared to such half-life of fragment CFTR³⁸⁰ in a cell expressing the fragment in the absence of the corrector. In some embodiments, the accumulation of NBD1 fragment, ΔF508-NBD1 fragment, fragment CFTR³⁷⁵ and/or fragment CFTR³⁸⁰ is determined by Western Blot.

In some embodiments, the corrector agent used in the methods and compositions of the invention is characterized in vitro by an ability to increase chloride transport in the presence of the corrector in one or more of the following CFTR mutations: E56K, P67L, E92K, L206W and/or ΔF508.

In some embodiments, the corrector agent used in the methods and compositions of the invention is characterized in vitro by a similar increase in accumulation of fragment CFTR³⁷⁰ or half-life of fragment CFTR³⁷⁰ in the presence of the corrector compared to such accumulation of fragment CFTR³⁷⁰ or half-life of fragment CFTR³⁷⁰, respectively, in the absence of the corrector.

In some embodiments, the corrector agent used in the methods and compositions of the invention does not increase accumulation of a C-form in a fragment CFTR³⁸⁰ containing a mutation or deletion between residues 362-380.

In some embodiments, proteolysis of fragment CFTR³⁸⁰ by trypsin in the presence of a corrector agent of this invention produces an increased amount of a 22 kD protease resistant fragment. In some embodiments, the corrector agent is capable of increasing the amount of a protease resistant 22 kD fragment produced by the proteolysis of full-length ΔF508 CFTR in the presence of the corrector agent. In some embodiments, a wildtype or mutant CFTR protein in the presence of the corrector agent in vitro is at least 100%, 200% or 250% more resistant to proteolysis than the wildtype or mutant CFTR protein in the absence of the corrector agent in vitro. In some embodiments, the proteolysis resistance observed is the proteolysis resistance of NBD2 in the wildtype or mutant CFTR protein. In some embodiments, the proteolysis resistance is trypsin resistance. In some embodiments, the proteolysis resistance is V8 protease resistance.

In some embodiments, the corrector agent used in the methods and compositions of the invention is capable of promoting interaction between MSD1 and NBD1 in a wildtype or mutant CFTR protein. In some embodiments, the interaction between MSD1 and NBD1 is between intracellular loop 1 (ICL1) and NBD1. In some embodiments, the corrector agent is capable of interacting with MSD1 prior to the synthesis of NBD1. In some embodiments, the corrector agent does not bind MSD2. In some embodiments, the corrector agent is capable of promoting interaction between ICL4 and NBD1. In some embodiments, the corrector agent is capable promoting the interaction in vitro.

In some embodiments, the corrector agent used in the methods and compositions of the invention is capable of selectively interacting with a full-length CFTR protein or a fragment thereof, wherein the fragment thereof comprises MSD1. In some embodiments, the corrector agent is not capable of interacting with any of an ion channel other than CFTR, an ABC transporter other than CFTR, a misfolded protein other than mutant CFTR, a G-protein coupled receptor, a kinase, a molecular chaperone, an ER stress marker and activation marker.

In some embodiments, a wildtype or mutant CFTR protein in the presence of the corrector agent in vitro is less susceptible to ER associated degradation (ERAD) than is the wildtype or mutant CFTR protein in the absence of the corrector agent in vitro. In some embodiments, the wildtype or mutant CFTR protein in the presence of the corrector agent in vitro is less susceptible to degradation by a proteasome than is the wildtype or mutant CFTR protein in the absence of the corrector agent in vitro. In some embodiments, the susceptibility to ER associated degradation (ERAD) of a mutant CFTR protein in the presence of the corrector agent in vitro is more similar to the susceptibility to ERAD of a wildtype CFTR than to the susceptibility to ERAD of the mutant CFTR protein in the absence of the corrector agent in vitro. In some embodiments, the susceptibility to degradation by a proteasome of a mutant CFTR protein in the presence of the corrector agent in vitro is more similar to the susceptibility to degradation by a proteasome of a wildtype CFTR protein than to the susceptibility to degradation by a proteasome of the mutant CFTR protein in the absence of the corrector agent in vitro.

In some embodiments, the method of the invention further comprises the step of administering to the patient one or more additional therapeutic agents, wherein the additional therapeutic agent is a CFTR potentiator. In some embodiments, the CFTR potentiator is ivacaftor or a pharmaceutically acceptable salt thereof. In some embodiments, the wildtype or mutant CFTR protein is capable of being potentiated by ivacaftor. In some embodiments, ivacaftor and the corrector agent are administered to the patient orally.

In some embodiments, the method further comprises the step of administering to the patient one or more additional therapeutic agents, wherein the additional therapeutic agent is selected from the group consisting of a bronchodilator, an antibiotic, a mucolytic agent, a nutritional agent and an agent that blocks ubiquitin-mediated proteolysis. In some embodiments, the additional therapeutic agent is an agent that blocks ubiquitin-mediated proteolysis. In some embodiments, the agent that blocks ubiquitin-mediated proteolysis is a proteasome inhibitor. In some embodiments, the agent that blocks ubiquitin-mediated proteolysis is selected from the group consisting of a peptide aldehyde, a peptide boronate, a peptide α′β′-epoxyketone, a peptide ketoaldehyde or a β-lactone. In some embodiments, the agent that blocks ubiquitin-mediated proteolysis is selected from the group consisting of bortezomib, carfilzomib, marizomib, CEP-18770, MLN-9708 and ONX-0912.

In some embodiments, the corrector agent and the one or more additional therapeutic agents are concurrently administered to the patient. In some embodiments, the corrector agent and the one or more additional therapeutic agents are administered consecutively to the patient. In some embodiments, the corrector agent and the one or more additional therapeutic agents are administered sequentially to the patient. In some embodiments, the corrector agent and the one or more additional therapeutic agents are administered to the patient in a single formulation. In some embodiments, the corrector agent and the one or more additional therapeutic agents are administered to the patient in separate formulations.

In some embodiments, the patient treated with the method of the invention has a mutant CFTR protein, wherein the mutant CFTR protein comprises a mutation in the MSD1 domain of the CFTR protein.

In some embodiments, the mutant CFTR protein comprises a mutation in any one of or combination of the transmembrane 1 (TM1), TM2, TM3, TM4, TM5 or TM6 domains. In some embodiments, the mutant CFTR protein comprises a mutation at an amino acid position corresponding to amino acid residue 92 of SEQ ID NO: 1. In some embodiments, the mutant CFTR protein comprises a mutation selected from the group consisting of a substitution of lysine, glutamine, arginine, valine or aspartic acid for glutamic acid at amino acid residue 92 of SEQ ID NO: 1. In some embodiments, the mutant CFTR protein comprises a mutation at an amino acid position corresponding to amino acid residue 139 of SEQ ID NO: 1. In some embodiments, the mutant CFTR protein comprises a substitution of arginine for histidine at amino acid residue 139 of SEQ ID NO: 1. In some embodiments, the mutant CFTR protein comprises a mutation at the amino acid position corresponding to amino acid residue 206 of SEQ ID NO: 1. In some embodiments, the mutant CFTR protein comprises a substitution of leucine for tryptophan at amino acid residue 206 of SEQ ID NO:1.

In some embodiments, the patient has a mutant CFTR protein, wherein the mutant CFTR protein comprises a mutation in a coupling helix extending from transmembrane 2 (TM2) region or transmembrane 3 (TM3) region of the CFTR protein. In some embodiments, the mutant CFTR protein comprises a mutation at an amino acid position corresponding to amino acid residue 149 or 192 of SEQ ID NO: 1.

In some embodiments, the patient has a mutant CFTR protein, wherein the mutant CFTR protein comprises a mutation in the nuclear binding domain 1 (NBD1) domain of CFTR protein. In some embodiments, the mutant CFTR protein comprises a deletion of phenylalanine at amino acid residue 508 of SEQ ID NO: 1.

In some embodiments, the corrector agent used in the methods and compositions of the invention is a non-naturally occurring agent. In some embodiments, the corrector agent is a polypeptide corrector agent. In some embodiments, the corrector agent is an antibody or antibody fragment. In other embodiments, the corrector agent is a small molecule.

In some embodiments, the corrector agent used in the methods and compositions of the invention is formulated with a pharmaceutically acceptable carrier. In some embodiments, the corrector agent is administered to the patient orally, sublingually, intravenously, intranasally, subcutaneously or intra-muscularly. In some embodiments, the corrector agent is orally administered to the patient.

In another aspect, the invention relates to a method of screening for a candidate corrector agent comprising the steps of: a) contacting a test agent with a cell expressing a CFTR fragment, wherein the CFTR fragment is a fragment CFTR³⁷⁵ or a fragment CFTR³⁸⁰, b) measuring the accumulation of the CFTR fragment in the cell, and c) comparing the accumulation of the CFTR fragment in the cell with the accumulation of the CFTR fragment in a cell not contacted with the test agent, wherein if the accumulation of CFTR fragment in the cell contacted with the test agent is greater than the accumulation of CFTR fragment in the cell not contacted with the test agent, the test agent is a candidate corrector agent. In some embodiments, the candidate corrector agent is a corrector agent.

In some embodiments, the method of screening for a candidate corrector agent comprises the steps of: a) contacting a test agent with a cell expressing a CFTR fragment, wherein the CFTR fragment is an NBD1 fragment, a ΔF508-NBD1 fragment, a CFTR³⁷³ fragment, or a CFTR³⁷⁰ fragment, b) measuring the accumulation of the CFTR fragment in the cell, and c) comparing the accumulation of the CFTR fragment in the cell with the accumulation of the CFTR fragment in a cell not contacted with the test agent, wherein if the accumulation of CFTR fragment in the cell contacted with the test agent is greater than the accumulation of CFTR fragment in the cell not contacted with the test agent, the test agent is a candidate corrector agent. In some embodiments, the accumulation of CFTR fragment is determined by Western Blot.

In some embodiments, the method of screening for a candidate corrector agent comprises the steps of: a) contacting a test agent with a cell expressing a CFTR protein, b) measuring the amount of mature CFTR protein in the cell, c) comparing the amount of mature CFTR protein in the cell with the amount of the CFTR protein in a cell not contacted with the test agent, and, wherein if the amount of mature CFTR in the cell contacted with the test agent is greater than the amount of mature CFTR in the cell not contacted with the test agent, the test agent is a candidate corrector agent. In some embodiments, the amount of the mature CFTR protein is determined by Western Blot. In some embodiments, the candidate corrector agent is a corrector agent.

In some embodiments, the method of screening for a candidate corrector agent comprises the steps of: a) contacting a test agent with a cell expressing a mutant CFTR protein, b) measuring the amount or pattern of ubiquitination of the mutant CFTR protein in the cell, and c) comparing the amount or patterns of ubiquitination of the mutant CFTR protein in the cell with the ubiquitination pattern or amount of the mutant CFTR protein in a cell not contacted with the test agent, wherein if the amount or pattern of ubiquitination of the mutant CFTR protein in the cell contacted with the test agent is different than the amount or pattern of mutant CFTR protein in the cell not contacted with the test agent, the test agent is a candidate corrector agent.

In some embodiments, the method of screening for a candidate corrector agent comprises the steps of: a) contacting a test agent with a cell expressing a CFTR protein, b) measuring the ER export of the CFTR protein in said cell, and c) comparing the ER export of the CFTR protein in the cell contacted with the test agent with the ER export of the CFTR protein in a cell not contacted with the test agent, wherein if the ER export of the CFTR protein in the cell contacted with the test agent is greater than the ER export of the CFTR protein in the cell not contacted with the test agent, the test agent is a candidate corrector agent.

In some embodiments, the method of screening for a candidate corrector agent comprises the steps of: a) contacting a test agent with a cell expressing a CFTR protein, b) measuring the chloride transport of the CFTR protein in the cell, and c) comparing the chloride transport of the CFTR protein in the cell with the chloride transport of the CFTR protein in a cell not contacted with the test agent, wherein if the chloride transport of the CFTR protein in the cell contacted with the test agent is greater than the chloride transport of the CFTR protein in the cell not contacted with the test agent, the test agent is a candidate corrector agent. In some embodiments, the chloride transport is determined by measuring ion flow across cell membranes of cells expressing the CFTR protein. In some embodiments, the measurement of ion flow is performed by utilizing Ussing chamber recording analysis. In some embodiments, the candidate corrector agent is a corrector agent.

In some embodiments, the method of screening for a candidate corrector agent comprises the steps of: a) contacting a test agent with a cell expressing a CFTR protein, b) measuring the CFTR protein channel gating in the cell, and c) comparing the CFTR protein channel gating in the cell with the CFTR protein channel gating in a cell not contacted with the test agent, wherein if the channel gating of the CFTR protein in the cell contacted with the test agent is greater than the channel gating of the CFTR protein in the cell not contacted with the test agent, the test agent is a candidate corrector agent. In some embodiments, the amount of channel gating is determined by single-channel patch clamp recording analysis. In some embodiments, the candidate corrector agent is a corrector agent.

In some embodiments, the method of screening for a candidate corrector agent comprises the steps of: a) contacting a test agent with a cell expressing a CFTR protein, b) measuring the ATPase activity of the CFTR protein in the cell, and c) comparing the ATPase activity of the CFTR protein in the cell with the ATPase activity of the CFTR protein in a cell not contacted with the test agent, wherein if the ATPase activity of the CFTR protein in the cell contacted with the test agent is greater than the ATPase activity of the CFTR protein in the cell not contacted with the test agent, the test agent is a candidate corrector agent. In some embodiments, the candidate corrector agent is a corrector agent.

### BRIEF DESCRIPTION OF THE DRAWINGS

FIG. 1 shows the effect of lumacaftor on the N-terminal regions of CFTR. FIG. 1A is a diagram depicting the domain boundaries of CFTR fragments used in this study. FIG. 1B shows the impact of bortezomib (Bort, 10 μM) or vehicle (DMSO) on the steady state levels of N-terminal CFTR fragments (CFTR³⁷⁰, CFTR⁴³⁰, CFTR⁵³⁰, CFTR⁶⁵³ and ΔF508-CFTR⁶⁵³) expressed in HEK293 cells. Indicated CFTR fragments were generated via introduction of a stop codon after the specified residue. Respective fragments were detected by western blot with an antibody against the N-terminal tail of CFTR. FIGS. 1C and 1D show the impact of lumacaftor (5 μM) on the steady state levels of N-terminal CFTR fragments (CFTR³⁷⁰, CFTR³⁷³, CFTR³⁷⁵, CFTR³⁸⁰, CFTR⁶⁵³ and ΔF508-CFTR⁶⁵³). Tubulin was used as a negative control. FIG. 1E shows the dose dependence of lumacaftor action on CFTR³⁸⁰. Tubulin was used as a negative control. FIG. 1F shows the results of a pulse-chase analysis testing the impact of lumacaftor on the half-life of CFTR³⁸⁰. Levels of ³⁵S-CFTR fragments were measured at 0, 30, 60, 90 and 120 minutes following administration of lumacaftor or vehicle (DMSO). Data are representative of 3 experiments.

FIG. 2 shows the effect of lumacaftor on the accumulation of different N-terminal regions of CFTR. FIG. 2A is a series of Western Blots showing the effect of lumacaftor and vehicle on accumulation levels of several N-terminal CFTR fragments (CFTR²⁶⁰, CFTR³⁷⁰, CFTR⁴⁰⁰, CFTR⁴³⁸, CFTR⁶⁴², CFTR⁸³⁷, CFTR¹¹⁷², ΔF508-CFTR⁶⁴², ΔF508-CFTR⁸³⁷, and ΔF508-CFTR¹¹⁷²). FIG. 2B shows the results of a pulse-chase analysis in which the effect of 5 μM lumacaftor or vehicle on the half-life of several CFTR fragments and variants (i.e., CFTR³⁷⁰, CFTR³⁷⁵, CFTR⁴³⁰, CFTR⁶⁵³ and ΔF508-CFTR⁶⁵³) was assessed.

FIG. 3 shows a comparison of trypsin resistant fragments liberated from CFTR³⁸⁰ and Δ508-CFTR in the presence or absence of lumacaftor. CFTR³⁸⁰ and ΔF508-CFTR were expressed in HEK293 cells in the absence or presence of lumacaftor (5 μM). Cells were incubated 18 hrs in the presence of lumacaftor and lysed with phosphate buffered saline that contained 1% triton X-100. The indicated concentration of trypsin was added and digestions were carried out on ice for 15 minutes. CFTR was detected via western blot with an N-terminal tail antibody. The asterisk indicates a band that corresponds to a protease-resistant species with an apparent molecular weight of 22 Kd.

FIG. 4 shows the role of amino acids 370-380 of CFTR on lumacaftor activity. Amino acids 370-380 of CFTR are required for folding of MSD1 to a conformation that is stabilized by lumacaftor. FIG. 4A is a Western Blot showing the effect of various concentrations of lumacaftor and DMSO on accumulation of B and C forms of CFTR protein lacking amino acid residues 371-375. Tubulin was used as a negative control. FIG. 4B (upper panel) shows the effect of lumacaftor on accumulation of CFTR³⁸⁰ and on CFTR³⁸⁰ lacking amino acid residues 371-375. FIG. 4B (middle panel) shows the effect of lumacaftor (5 μM) on levels of CFTR³⁸⁰ and CFTR³⁸⁰ having an F374A mutation. FIG. 4B (lower panel) shows the effect of lumacaftor (5 μM) on levels of CFTR³⁸⁰ having an F375A mutation. FIG. 4C shows a comparison of trypsin resistant fragments liberated from CFTR³⁷⁰ (upper panel) and CFTR³⁸⁰ (lower panel) at various time points in the presence and absence of (5 μM) lumacaftor. The different trypsin concentrations used in this experiment are indicated. Cells were lysed with phosphate buffered saline that contained 1% Triton X-100 and the indicated concentration of trypsin and digestions were carried out on ice for 15 minutes. A single asterisk indicates either the full-length CFTR³⁷⁰ or CFTR³⁸⁰ fragment, and a double asterisk is the predicted molecular weight of the protease resistant fragment noted in FIG. 3. FIG. 4D shows the effect of F374A and L375A mutations on steady state levels of the C-form of CFTR in the presence of (5 μM) lumacaftor or DMSO vehicle control. Tubulin was used as a negative control. Levels of indicated forms of CFTR expressed in HEK293 cells were detected by Western Blot with an N-terminal tail antibody.

FIG. 5 shows the effect of lumacaftor on thermal stability of isolated NBD1. Lumacaftor (i.e., VX-809) does not influence the thermal stability of isolated NBD1. Purified mouse NBD1 (1.5 μM) was incubated with DMSO (solid lines) or 25 μM lumacaftor (dashed lines) in the absence or presence of 2 mM ATP and 5×SYPRO orange. The temperature was then increased from 10 to 80° C. (speed of increase 0.5° C./min) in a BioRad CFX384 rdPCR machine, and the fluorescence increase of the SYPRO dye was recorded as a readout of protein unfolding. First derivative (dRFU/dT) plots are shown for Tm values, and were approximated by using the inflection point (i.e. the maximum of each 1^{st} derivative plot) of each trace of six independent samples for each condition. Plots show the averaged traces for simplicity. Lumacaftor does not influence the thermal stability of isolated NBD1.

FIG. 6 shows the effect of lumacaftor on the folding and function of various CFTRs having disease-related mutations in the N-terminus. Functional defects in CFTR caused by disease related mutations in MSD1 are suppressed by lumacaftor. FIG. 6A shows the effect of lumacaftor on the levels of B- and C-forms of WT, E56K, P67L, E92K, L206W and V232D CFTR mutants. Tubulin was used as a negative control. FIG. 6B shows the effect of various concentrations of lumacaftor on B- and C-form levels of the E92K CFTR mutant in the presence or absence of the ΔF508 mutation. FIG. 6C shows the effect of lumacaftor on chloride transport of E92K-CFTR and ΔF508-CFTR as measured in forskolin (10 μM) stimulated cells using an USSING chamber (N=3−/+ standard error). FIG. 6D shows that lumacaftor (30 μM) and Corr4a (15 μM) restore chloride transport of E92K-CFTR to different levels, as measured in forskolin (10 μM) stimulated cells using an USSING chamber (N=3−/+ standard error). FIG. 6E shows the effect of lumacaftor or vehicle (DMSO) on the function of different CFTR mutants E56K, P67L, E92K, L206W, V232D and ΔF508 as measured in forskolin (10 μM) stimulated cells using an USSING chamber chloride transport assay.

FIG. 7 shows the effect of a mutation of E92 to a different amino acid on the correction of E92-mutant folding and function by lumacaftor. FIG. 7A shows a dose response of E92K, E92Q, E92D, E92A, E92V, and E92R mutant folding to lumacaftor. The Western Blots show the level of the B- and C-form of CFTR in cell extracts. FIG. 7B shows the effect of lumacaftor on chloride transport of E92K, E92Q, E92D, E92A, E92V, and E92R mutant CFTRs. cAMP stimulated CFTR activity in polarized FRT cells was measured in USSING chambers.

FIG. 8 shows that interdomain interaction between MSD1 and NBD1 is required for lumacaftor to enhance biosynthetic processing of CFTR. FIG. 8A shows that the mutation of F374A hinders the ability of misfolding suppressor mutations S2 and S3 to increase the efficacy of lumacaftor on ΔF508-CFTR. FIG. 8B shows that the mutation of F374A hinders the ability of S2 and S3 to increase accumulation of the folded C-form of CFTR. Data in panels are from Western Blots of cell extracts. Panels are a representation of 3 experiments. Quantitation is normalized to 100% of total C-form for CFTR detected for wild type. Tubulin was used as a negative control.

FIG. 9 shows that the restoration of contact between ΔF508-NBD1 and ICL4 increases the efficacy of lumacaftor in the repair of ΔF508-CFTR misfolding. FIG. 9A shows the effect of lumacaftor on CFTR¹⁻⁸³⁷ and CFTR⁸³⁷⁻¹⁴⁸⁰ fragments, and on fragments having or lacking the ΔF508-CFTR mutation. FIG. 9B shows the effect of the introduction of the V510D suppressor mutation into NBD1 on levels of ΔF508-CFTR in the presence or absence of lumacaftor. Panels A and B are Western Blots using an N-terminal tail CFTR antibody. Lumacaftor was present at 5 μM. Tubulin was used as a negative control.

FIG. 10 shows the effect of lumacaftor and an active photoanalog of lumacaftor on ΔF508-CFTR and on an MSD1 fragment (amino acids 1-437 of SEQ ID NO: 1). FIG. 10A depicts a representative Western Blot showing the effect of increasing dosage levels of lumacaftor, an active photoanalog and an inactive analog of lumacaftor on accumulation levels of the C-form of ΔF508-CFTR. FIG. 10B depicts a representative Western Blot showing the effect of lumacaftor (10 μM), an active photoanalog (10 μM) and an inactive photoanalog (10 μM) on steady state levels of an MSD1 fragment. GAPDH was used as a negative control.

FIG. 11 shows a molecular weight profile of proteins labeled with the active photoanalog of lumacaftor. Sf9 whole cell lysates were separated on 4-12% Bis-Tris gel. The gel (“Gel”) was then either cut into different molecular weight range (“MW Range”) fragments and counted in a liquid scintillation counter, or first transferred to a nitrocellulose membrane (“Membrane”) and then cut from the membrane and counted in a liquid scintillation counter. Counts per minute are indicated for protein samples treated with tritiated active photoanalog (“³H-Act.”), with ³H-Act. plus non-tritiated active photoanalog (“Act.”), or with ³H-Act. plus non-tritiated inactive photoanalog (“Inact.”).

FIG. 12 shows the binding of a tritiated active photoanalog of lumacaftor to MSD1 expressed in HEK293 cell lysates. FIG. 12A shows a diagram of the MSD1 construct used in the binding experiments. The construct included a 2× Hemagluttinin tag (HA) and a histidine tag (His₆) as well as the N-terminal 438 amino acids of the CFTR protein. The 438 amino acids include the full MSD1 domain (including the linker region between TM6 and NBD1) and the regulatory insert (RI), a 32-residue segment within the NBD1 domain. FIG. 12B (upper panel) shows the binding affinity between the active photoanalog and a control polypeptide or the MSD1 construct in the presence or absence of twenty-fold excess non-tritiated (cold) active photoanalog or cold inactive lumacaftor analog. FIG. 12B (lower panel) shows the levels of MSD1 expressed in the HEK293 cell lysates used in the binding experiments (upper panel). The MSD1 construct was immunoprecipitated using an anti-HA antibody and detected by immunoblot using an anti-RI antibody. ***P<0.001, 2-way ANOVA.

FIG. 13 shows the binding of a tritiated active photoanalog of lumacaftor to MSD1 expressed in live Sf9 cells. FIG. 13A shows a diagram of the MSD1 construct used in the binding experiments. The construct included 2× Hemagluttinin tags (HA) and a histidine tag (His₆) as well as the N-terminal 438 amino acids of the CFTR protein. The 438 amino acids include the full MSD1 domain (including the linker region between TM6 and NBD1) and the regulatory insert (RI), a 32-residue segment within the NBD1 domain. FIG. 13B (upper panel) shows the binding affinity between the active photoanalog and a control polypeptide (CFTR amino acids 837-1172 of SEQ ID NO:1) or the MSD1 construct in the presence or absence of twenty-fold excess non-tritiated (cold) active photoanalog or cold inactive lumacaftor analog. FIG. 13B (lower panel) shows the levels of MSD1 expressed in the Sf9 cell lysate used in the binding experiments (upper panel). The MSD1 construct was detected by immunoblot using an anti-RI antibody. *** P<0.001, 2-way ANOVA.

FIG. 14 shows the selective binding of an active photoanalog of lumacaftor to MSD1 in a dose-dependent manner. FIG. 14A is a graph showing the dose-dependent binding of the active photoanalog to the MSD1 construct. FIG. 14B shows a Western Blot that indicates the amount of MSD1 and MSD2 for each of the different concentrations of active photoanalog tested in FIG. 14A.

FIG. 15 shows the selective binding of a lumacaftor active photoanalog to MSD1 in a dose-dependent manner. FIG. 15A shows a diagram of the MSD1 and MSD2 constructs used in the binding experiments. The MSD1 construct contains the N-terminal 438 amino acids of CFTR, which include the full MSD1 domain (including the linker region between TM6 and NBD1) and the regulatory insert (RI), a 32-residue segment within the NBD1 domain. The MSD2 construct contains amino acids 837-1162, which includes the full MSD2 domain. FIG. 15B is a graph showing the dose-dependent binding of the lumacaftor active photoanalog to the MSD1 construct.

FIG. 16 shows that lumacaftor and the active photoanalog of lumacaftor interact with CFTR-MSD1 fragments lacking the RI region. Cells expressing the 438X or 392X MSD1 fragments or a control vector were treated with 1 μM tritiated active photoanalog (high specific activity) and with DMSO, 20 μM non-tritiated (cold) active photoanalog, 20 μM cold inactive photoanalog, or 20 μM cold lumacaftor. FIG. 16A shows the effect of cold active lumacaftor analog or cold lumacaftor on binding between the different MSD1 fragments and the tritiated active lumacaftor photoanalog. FIG. 16B shows Western Blot analysis of the MSD1 fragment expression levels in Sf9 cells from samples tested in FIG. 16A.

FIG. 17 shows that lumacaftor and the active lumacaftor photoanalog interact with CFTR-MSD1 fragments lacking the RI region. FIG. 17A shows a diagram of the MSD1 fragments (CFTR³⁷⁶, CFTR³⁸⁵, CFTR³⁹², CFTR⁴³⁸) used in the binding experiments. Only the CFTR⁴³⁸ fragment includes the RI region. FIG. 17B shows the effect of non-tritiated (cold) active lumacaftor analog or cold lumacaftor on binding between the different MSD1 fragments and the active lumacaftor photoanalog. FIG. 17C shows the effect of lumacaftor on levels of ΔF508-CFTR protein possessing or lacking the RI region. GAPDH was used as a negative control. * P<0.05, *** P<0.001, 2-way ANOVA.

FIG. 18 shows that lumacaftor competes with the active lumacaftor photoanalog for MSD1 binding in a concentration-dependent manner. Mock-transfected Sf9 cells or Sf9 cells expressing the CFTR⁴³⁸ fragment were cultured in the presence of 1 μM active photoanalog plus 3, 10, or 20 μM of non-tritiated (cold) lumacaftor or a cold inactive analog of lumacaftor. Sf9 whole cell lysates were separated on 4-12% Bis-Tris gel, and the specified molecular weight range (either 10-15 kDa or 35-42 kDa) was cut from the gel and counted using a liquid scintillation counter. The 10-15 kDa MW range was included as a negative control.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Each embodiment of the invention described herein may be taken alone or in combination with one or more other embodiments of the invention.

Throughout this specification, the word “comprise” or variations such as “comprises” or “comprising” will be understood to imply the inclusion of a stated integer or groups of integers but not the exclusion of any other integer or group of integers.

Throughout this specification, the word “a” will be understood to imply the inclusion of one or more of the integers modified by the article “a.”

In order to further define the invention, the following terms and definitions are provided herein.

### DEFINITIONS

As used herein, “antibody fragment” is understood to include a bioactive fragment or bioactive variant that exhibits “bioactivity” as described herein. That is, a bioactive fragment act through MSD1 during biosynthesis of a CFTR protein.

As used herein, “B-form” refers to a core-glycosylated CFTR protein or CFTR protein fragment that is endoH-sensitive and corresponds to nascent CFTR that has not been processed by mannosidases in the cis/medial Golgi endoH-resistant oligosaccharide chains.

As used herein, the term “C-form” refers to CFTR protein or CFTR protein fragment that is fully glycosylated and resistant to digestion with endoH and that is presumed to have trafficked at least to the cis/medial cisternae of the Golgi apparatus.

As used herein, the term “CFTR” or “CFTR protein” refers to a protein having at least 50%, 60%, 70%, 80%, 90%, 91%, 92%, 93%, 94%, 95%, 96%, 97%, 98%, 99% or 100% sequence identity to the sequence of SEQ ID NO: 1, or a fragment thereof. Unless specifically stated otherwise, the term “CFTR” or “CFTR protein” encompasses wildtype and mutant CFTR proteins.

As used herein, “ER export” refers to the transport of a protein out of the ER, e.g., by vesicles, to at least the Golgi apparatus.

As used herein, a “non-naturally occurring” corrector agent refers to an agent that is not produced by a cell, organism, animal or plant in the absence of human manipulation.

A “patient,” “subject” or “individual” are used interchangeably and refer to either a human or non-human animal. The term includes mammals such as humans.

The terms “effective dose” or “effective amount” are used interchangeably herein and refer to that amount that produces the desired effect for which it is administered (e.g., improvement in CF or a symptom of CF or lessening the severity of CF or a symptom of CF). The exact amount will depend on the purpose of the treatment, and will be ascertainable by one skilled in the art using known techniques (see, e.g., Lloyd (1999) The Art, Science and Technology of Pharmaceutical Compounding).

As used herein, the term “MSD1,” unless specified otherwise, refers to the portion of the CFTR protein that includes the TM1-TM6 regions (i.e., amino acids 83-358), as well as the linker region between TM6 and NBD1 (i.e., amino acids 359-388 of SEQ ID NO: 1).

As used herein, the term “mutant CFTR” means that the CFTR protein has at least one amino acid mutation as compared to a wildtype CFTR protein. Mutations include amino acid insertions, deletions and substitutions.

As used herein, the terms “treatment,” “treating,” and the like generally mean the improvement of CF or its symptoms or lessening the severity of CF or its symptoms in a subject. “Treatment,” as used herein, includes, but is not limited to, the following: increased growth of the subject, increased weight gain, reduction of mucus in the lungs, improved pancreatic and/or liver function, reduced cases of chest infections, and/or reduced instances of coughing or shortness of breath. Improvements in or lessening the severity of any of these conditions can be readily assessed according to standard methods and techniques known in the art.

As used herein, the term “wildtype CFTR” means a CFTR protein having the sequence of SEQ ID NO: 1.

The invention provides methods of treating CF in a subject, e.g., a human patient, by administering to the subject a corrector agent, as defined herein, capable of acting through MSD1 during the biosynthesis of a CFTR protein. The invention also provides methods of screening for and identifying new corrector agents, as defined herein, capable of acting through MSD1 during the biosynthesis of a CFTR protein. Further, the invention provides pharmaceutical compositions comprising a corrector agent, as defined herein, capable of acting through MSD1 during the biosynthesis of a CFTR protein.

### A. The Corrector Agents

The corrector agent of the present invention is capable of modulating a wildtype or mutant CFTR protein in vitro in each of the following ways: a) increasing chloride transport of the wildtype or mutant CFTR protein, b) decreasing proteolytic sensitivity of the wildtype or mutant CFTR protein, c) increasing trafficking of the wildtype or mutant CFTR protein out of the ER (i.e., increasing ER export), and d) increasing the amount of functional wildtype or mutant CFTR at the cell surface. In addition, a corrector agent of the present invention is capable of acting through the membrane spanning domain 1 (MSD1) during the biosynthesis of a wildtype or mutant CFTR protein (e.g., on nascent CFTR translation intermediates), wherein the action is characterized in vitro by one or more of the following: (i) an increase in accumulation of fragment CFTR³⁷⁵ in a cell expressing the fragment in the presence of the corrector compared to such accumulation of fragment CFTR³⁷⁵ in a cell expressing the fragment in the absence of the corrector, (ii) an increase in accumulation of fragment CFTR³⁸⁰ in a cell expressing the fragment in the presence of the corrector compared to such accumulation of fragment CFTR³⁸⁰ in a cell expressing the fragment in the absence of the corrector, (iii) an increase in the half-life of fragment CFTR³⁷⁵ in a cell expressing the fragment in the presence of the corrector compared to such half-life of fragment CFTR³⁷⁵ in a cell expressing the fragment in the absence of the corrector, (iv) an increase in the half-life of fragment CFTR³⁸⁰ in a cell expressing the fragment in the presence of the corrector compared to such half-life of fragment CFTR³⁸⁰ in a cell expressing the fragment in the absence of the corrector, (v) an increase in the half-life of fragment CFTR³⁸⁰, CFTR⁴³⁰, and/or CFTR⁶⁵³ in a cell expressing CFTR³⁸⁰, CFTR⁴³⁰, and/or CFTR⁶⁵³ in the presence of said corrector compared to the half-life of CFTR³⁸⁰, CFTR⁴³⁰, and/or CFTR⁶⁵³, respectively, in a cell expressing said fragment in the absence of said corrector, or (vi) an enhanced resistance of fragment CFTR³⁸⁰ to proteolysis with trypsin in the presence of the corrector compared to such proteolysis in the absence of the corrector. In some embodiments, the corrector agent is characterized by one, two, three, four, five, six or seven characteristics selected from characteristics (i)-(vi). In some embodiments, the concentration of said corrector agent needed to achieve the maximal accumulation of fragment CFTR³⁸⁰ in a cell expressing said fragment is about the same concentration of said corrector agent needed to achieve the maximal accumulation of full-length CFTR in a cell expressing said full-length CFTR. In some embodiments, the increases in half-life values for fragments CFTR³⁸⁰, CFTR⁴³⁰, and/or CFTR⁶⁵³ in a cell expressing said fragment CFTR³⁸⁰, CFTR⁴³⁰, and/or CFTR⁶⁵³ in the presence of said corrector are comparable to the increases in half-life values for fragments CFTR³⁸⁰, CFTR⁴³⁰, and CFTR⁶⁵³ in a cell expressing said fragment CFTR³⁸⁰, CFTR⁴³⁰, and/or CFTR⁶⁵³ in the absence of said corrector,

The corrector agent of the present invention is not a proteasome inhibitor or any of the compounds disclosed in U.S. Pat. No. 7,407,976; U.S. Pat. No. 7,645,789; U.S. Pat. No. 7,659,268; U.S. Pat. No. 7,671,221; U.S. Pat. No. 7,691,902; U.S. Pat. No. 7,741,321; U.S. Pat. No. 7,754,739; U.S. Pat. No. 7,776,905; U.S. Pat. No. 7,973,169; U.S. Pat. No. 7,977,322; U.S. Pat. No. 7,999,113; U.S. Pat. No. 8,227,615; U.S. Pat. No. 8,299,099; US Published Application No. 2006-0052358; US Published Application No. 2009-0143381; US Published Application No. 2009-0170905; US Published Application No. 2009-0253736; US Published Application No. 2011-0263654; or US Published Application No. 2011-0251253, PCT Application No. WO2008141119, U.S. application Ser. No. 13/672,538 and U.S. application Ser. No. 11/047,361, the disclosure of each of which is incorporated herein by reference.

The corrector agent of the present invention is not any of the compounds disclosed in Table 1.

TABLE 1
Compounds disclosed in U.S. Pat. No. 7,407,976 (Col 6, ln 12-col 66, ln 67; col 138, ln
32-col 145, ln 5; Table 1)
Compounds disclosed in U.S. Pat. No. 7,645,789 (Col 16, ln 52-col 50, ln 22; col 167, ln
64-col 213, ln 50; col 222, ln 1-col 495, ln 43; Table 1)
Compounds disclosed in U.S. Pat. No. 7,659,268 (Col 16, ln 20-col 70, ln 52; col 349, ln
6-col 502, ln 67; Table 1)
Compounds disclosed in U.S. Pat. No. 7,671,221 (Col 16, ln 12-col 54, ln 48; col 710, ln
55-col 774, ln 67; Table 1)
Compounds disclosed in U.S. Pat. No. 7,691,902 (Col 16, ln 11-col 54, ln 29; col 695, ln
17-col 749, ln 36; Table 1)
Compounds disclosed in U.S. Pat. No. 7,741,321 (Col 16, ln 21-col 72, ln 17; col 290, ln
40-col 367, ln 10; Table 1)
Compounds disclosed in U.S. Pat. No. 7,754,739 (Col 16, ln 1-col 22, ln 47; col 30, ln
57-col 34, ln 67)
Compounds disclosed in U.S. Pat. No. 7,776,905 (Col 16, ln 23-col 38, ln 40; col 96, ln
42-col 107, ln 15; col 142, ln 15-col 374, ln 12; Table 1)
Compounds disclosed in U.S. Pat. No. 7,973,169 (Col 5, ln 30-col 7, ln 57; col 9, ln
15-col 40, ln 40; col 118, ln 57-col 152, ln 45; Table 1)
Compounds disclosed in U.S. Pat. No. 7,977,322 (Col 6, ln 26-col 37, ln 47; col 151, ln
10-col 206, ln 20; Table 1)
Compounds disclosed in U.S. Pat. No. 7,999,113 (Col 6, ln 13-col 34, ln 23; col 42, ln
44-col 97, ln 45)
Compounds disclosed in U.S. Pat. No. 8,227,615 (Col 6, ln 10-col 29, ln 66; col 61, ln
35-col 101, ln 41; Table 1)
Compounds disclosed in U.S. Pat. No. 8,299,099 (Col 6, ln 10-col 42, ln 35; col 55, ln
1-col 82, ln 47)
Compounds disclosed in US Published Application No. 2006-0052358 (Paragraphs [0034]-
[0056]; [0077]-[0241]; [0282]-[0421]; Table 1)
Compounds disclosed in US Published Application No. 2009-0143381 (Paragraphs [0101]-
[0264]; [0310]-[0393]; Table 1)
Compounds disclosed in US Published Application No. 2009-0170905 (Paragraphs [0012]-
[0013]; [0030]-[0070]; [0105]-[0148])
Compounds disclosed in US Published Application No. 2009-0253736 (Paragraphs [0031]-
[0163]; [0207]-[0268]; Table 1)
Compounds disclosed in US Published Application No. 2011-0263654 (Paragraphs [0012]-
[0013]; [0066]-[0141]; [0202]-[0250]; Table 1)
Compounds disclosed in US Published Application No. 2011-0251253 (Paragraphs [0012]-
[0013]; [0052]-[0079]; [0156]; [0173]-[0295]; Table 1)
Compounds disclosed in PCT application W02008141119 (Paragraphs [0024]-[0025],
[0100]-[0340]; [0404]-[0891]; Tables 1-3)
Compounds disclosed in US Application No. 11/047,361
Compounds disclosed in US Application No. 13/672,538

The corrector agent of the present invention includes, but is not limited to a small molecule, polypeptide, peptidomimetic, antibody, antibody fragment, antibody-like protein, and nucleic acid. In some embodiments, the corrector agent is a non-naturally occurring agent.

With respect to the corrector agent's ability to increase chloride transport of a CFTR protein, this may be determined by utilizing standard assays known in the art, including, but not limited to, the utilization of Ussing chamber recordings. Ussing chamber assays use electrodes to measure ion flow across the membranes of cells grown into a monolayer with tight junctions. See, e.g., Example 5. In some embodiments, ion flow is increased in a cell contacted with a corrector agent by at least 25%, 50%, 100%, 150%, 200%, 250%, 300%, 350%, 400%, 500%, 600%, 700%, 800%, 900%, or 1000% as compared to a control cell that is not contacted with the corrector agent. In some embodiments, the control cell is the same type of cell as the type of cell treated with the corrector agent.

Without being bound by theory, a corrector agent may increase chloride transport of a CFTR protein in a cell by increasing the CFTR protein channel gating, by increasing the amount of CFTR protein that is trafficked to the cell surface, or a combination thereof. In some embodiments, the corrector agent increases chloride transport by increasing the amount of CFTR protein that is trafficked to the cell surface. In some embodiments, the corrector agent increases chloride transport by both increasing the CFTR protein channel gating and by increasing the amount of CFTR protein that is trafficked to the cell surface. In some embodiments, the corrector agent action is characterized in vitro by an ability to increase chloride transport in the presence of the corrector in a CFTR containing one or more of the following mutations: E56K, P67L, E92K, L206W and/or ΔF508.

In some embodiments, the corrector agent increases chloride transport by increasing the CFTR protein channel gating. In some embodiments, the channel gating of a CFTR protein in the presence of the corrector agent is greater than the channel gating of the CFTR protein in the absence of the corrector agent. As used herein, “increasing CFTR channel gating” means increasing the open probability of a CFTR channel protein. In some embodiments, the channel gating of a mutant CFTR protein in the presence of the corrector agent is more similar to the channel gating of a wildtype CFTR protein than to the channel gating of the mutant CFTR protein in the absence of the corrector agent. Increases in channel gating may be determined by utilizing any one of numerous standard assays known in the art, including, but not limited to, the utilization of single-channel patch-clamp recording assays. Patch clamp recording assays measure the opening and closing rates of single channels, in which patches of the cell membrane are isolated using a micropipette tip and these patches are hooked up to microelectrodes. See, e.g., Example 6 and Devor et al., 2000, Am J Physiol Cell Physiol, 279(2): C461-79 and Dousmanis, et al., 2002, J Gen Physiol, 119(6): 545-59. In some embodiments, the corrector agent increases channel gating in a cell expressing a CFTR protein and contacted with a corrector agent by at least 50%, 100%, 150%, 200%, 250%, 300%, 350%, 400%, 500%, 600%, 700%, 800%, 900% or 1000% as compared to a control cell that expresses the CFTR but not treated with the corrector agent. In some embodiments, the control cell is the same type of cell as the type of cell treated with the corrector agent.

In some embodiments, the amount of CFTR protein trafficked to the cell surface in the presence of the corrector agent is greater than the amount of CFTR protein trafficked to the cell surface in the absence of the corrector agent. Cell surface CFTR may be isolated by a variety of means well-known in the art, including for example, the commercial Pierce Cell Surface Protein Isolation Kit (Thermo Fisher Scientific, Rockford, Ill.). Following isolation of membrane containing cell surface CFTR, the amounts of the cell surface CFTR in the membrane may be assessed by using an anti-CFTR antibody and assays such as, but not limited to, Western Blot or ELISA. Alternatively, cell surface CFTR amounts may be assessed by immunocytochemistry or immunohistochemistry. In some embodiments, a CFTR protein in the presence of the corrector agent is less susceptible to degradation at the cell surface than the CFTR protein in the absence of the corrector agent. In some embodiments, the susceptibility to degradation of a mutant CFTR protein at the cell surface in the presence of the corrector agent is more similar to the susceptibility to degradation of a wildtype CFTR protein at the cell surface than to the susceptibility to degradation of the mutant CFTR protein at the cell surface in the absence of the corrector agent.

With respect to the corrector agent's ability to decrease proteolytic sensitivity of the mutant CFTR protein, this may be determined by utilizing standard assays known in the art, including, but not limited to, an assay that assesses the amount of proteolysis of a mutant CFTR in the presence of carboxypeptidase, trypsin, V8 protease, papain or chymotrypsin and in the presence or absence of a corrector agent. In some embodiments, proteolysis resistance is determined by utilizing a standard proteolysis resistance assay (See, e.g., Example 7). In some embodiments, the amount of proteolysis of a mutant CFTR is determined by Western Blot. As determined by a utilizing a proteolytic resistance assay, a mutant CFTR protein in the presence of the corrector agent is more resistant to proteolysis during biosynthesis than the mutant CFTR protein in the absence of the corrector agent.

In some embodiments, the increased proteolytic resistance is of a nascent mutant CFTR translation intermediate. In some embodiments, the increased proteolytic resistance is of a full-length mutant CFTR protein. In some embodiments, the proteolysis resistance during biosynthesis of a mutant CFTR protein in the presence of the corrector agent is more similar to the proteolysis resistance during biosynthesis of a wildtype CFTR protein than to the proteolysis resistance during biosynthesis of the mutant CFTR protein in the absence of the corrector agent. In some embodiments, the corrector agent increases protease resistance of the full-length CFTR protein. In other embodiments, the corrector agent increases protease resistance of a fragment of the full-length CFTR protein (e.g., a translation intermediate). In some embodiments, the fragment of full-length CFTR protein is a fragment comprising at least MSD1. In some embodiments, the fragment of full-length CFTR protein is MSD1. In some embodiments, proteolysis resistance of a CFTR is increased in a cell contacted with a corrector agent by at least 25%, 50%, 100%, 150%, 200%, 250%, 300%, 350%, 400%, 500%, 600%, 700%, 800%, 900%, or 1000% as compared to a CFTR in a control cell that are not contacted with the corrector agent. In some embodiments, the control cell is the same type of cell as the type of cell treated with the corrector agent.

With respect to the corrector agent's ability to increase trafficking of the CFTR protein out of the ER (i.e., ER export), this may be determined by utilizing standard assays known in the art, including, but not limited to, a CFTR metabolic pulse-chase analysis. In such a pulse-chase analysis, cells expressing wildtype or mutant CFTR are treated with, or without, the test agent in the presence or absence of the ER-transport blocker, brefeldin A. At various intervals following treatment with the corrector agent, cells are harvested and the amount of immature CFTR is assessed. See, e.g., Example 3. In some embodiments, a corrector agent induces an increase in the amount of immature CFTR in a brefeldin A treated cell. In utilizing an ER trafficking assay, a CFTR protein in the presence of the corrector agent will be more endoplasmic reticulum (ER)-trafficking competent than the CFTR protein in the absence of the corrector agent. In some embodiments, ER-trafficking of a mutant CFTR protein in the presence of the corrector agent is more similar to the ER-trafficking of a wildtype CFTR protein than the ER-trafficking of the mutant CFTR protein in the absence of the corrector agent.

Another means by which trafficking of a mutant CFTR protein out of the ER may be assessed is to examine the amount of mature CFTR protein in a cell. Similar to other integral membrane glycoproteins, the initial stages of CFTR biosynthesis begin with the formation in the endoplasmic reticulum (ER) membrane of a core-glycosylated 135- to 140-kDa “immature” form that, if trafficked to the Golgi, is further modified to the “mature” 150- to 160-kDa CFTR that contains complex, endoH-resistant oligosaccharide chains (Kopito, R R, 1999, Physiol Rev, 79(1): S167-S173). As used herein, the term “mature CFTR,” in the context of full-length CFTR, refers to CFTR that migrates as a diffuse, 150- to 160-kDa band that is resistant to digestion with endoH and thus presumed to have trafficked at least to the cis/medial cisternae of the Golgi apparatus. The term “immature CFTR” refers to the 135- to 140-kDa, endoH-sensitive form corresponding to nascent CFTR that has not been processed by mannosidases in the cis/medial Golgi. As such, the amount of mature CFTR in a cell may be determined by performing a routine assay, such as a Western Blot, in order to determine the molecular weight of the CFTR protein present in the cell or sample from the subject. See, e.g., Example 2. In some embodiments, the mature CFTR is endoH-resistant.

In some embodiments, the corrector agent used in the methods and compositions of the invention is capable of increasing the amount of mature CFTR protein in a cell. In some embodiments, the amount of mature CFTR protein is greater in a cell in the presence of the corrector agent than the amount of mature CFTR protein in a cell in the absence of the corrector agent. In some embodiments, the amount of a mature mutant CFTR protein in a cell in the presence of the corrector agent is more similar to the amount of mature wildtype CFTR protein in a cell than to the amount of mature mutant CFTR protein in a cell in the absence of the corrector agent. In some embodiments, the corrector agent is an agent that, upon administration to a subject or upon contacting a cell having a CFTR protein, increases the amount of the mature CFTR protein such that the amount is at least 50%, 75%, 100%, 200%, 300%, 400%, 500%, 600%, 700%, 800%, 900%, or 1000% greater than the amount of mature CFTR protein in a cell prior to, or in the absence of, administration of the corrector agent.

In some embodiments, the corrector agent used in the methods and compositions of the invention is capable of reducing susceptibility of a mutant CFTR protein to ER-associated degradation (ERAD). ERAD is a cellular pathway which targets misfolded proteins of the endoplasmic reticulum for ubiquitination and subsequent degradation by a protein-degrading complex, called the proteasome. In some embodiments, a mutant CFTR protein in the presence of the corrector agent is less susceptible to ERAD than is the mutant CFTR protein in the absence of the corrector agent. In some embodiments, the susceptibility to ER associated degradation (ERAD) of a mutant CFTR protein in the presence of the corrector agent is more similar to the susceptibility to ERAD of a wildtype CFTR than to the susceptibility to ERAD of the mutant CFTR protein in the absence of the corrector agent. In some embodiments, the corrector agent is capable of reducing susceptibility of a mutant CFTR protein to degradation by a proteasome. In some embodiments, the mutant CFTR protein in the presence of the corrector agent is less susceptible to degradation by a proteasome than is the mutant CFTR protein in the absence of the corrector agent. In some embodiments, the susceptibility to degradation by a proteasome of the mutant CFTR protein in the presence of the corrector agent is more similar to the susceptibility to degradation by a proteasome of a wildtype CFTR protein than to the susceptibility to degradation by a proteasome of the mutant CFTR protein in the absence of the corrector agent.

In some embodiments, the corrector agent used in the methods and compositions of the invention is capable in vitro of increasing in accumulation of a fragment of the CFTR protein that includes at least the N-terminal 375 amino acids of a polypeptide having a sequence that is at least 80%, 85%, 90%, 91%, 92%, 93%, 94%, 95%, 96%, 97%, 98%, 99% or 100% identical to SEQ ID NO: 1 (i.e., a “fragment CFTR³⁷⁵⁺”) in a cell expressing the fragment in the presence of the corrector as compared to such accumulation of fragment CFTR³⁷⁵⁺ in a cell expressing the fragment in the absence of the corrector. In some embodiments, the fragment CFTR³⁷⁵⁺ is a “fragment CFTR³⁷⁵” (i.e., a fragment consisting of the N-terminal 375 amino acid residues of the full length CFTR protein—e.g., residues 1-375 of SEQ ID NO: 1), fragment CFTR³⁸⁰ (e.g., residues 1-380 of SEQ ID NO: 1), fragment CFTR³⁹⁰ (e.g., residues 1-390 of SEQ ID NO: 1), fragment CFTR⁴⁰⁰ (e.g., residues 1-400 of SEQ ID NO: 1), fragment CFTR⁴¹⁰ (e.g., residues 1-410 of SEQ ID NO: 1), fragment CFTR⁴²⁰ (e.g., residues 1-420 of SEQ ID NO: 1), fragment CFTR⁴³⁰ (e.g., residues 1-430 of SEQ ID NO: 1) or fragment CFTR⁶⁵³ (e.g., residues 1-653 of SEQ ID NO: 1). In some embodiments, the fragment CFTR³⁷⁵⁺ is a mutant fragment CFTR³⁷⁵⁺ (e.g., a fragment CFTR⁶⁵³ having a ΔF508 mutation). In some embodiments, the accumulation amount of the CFTR³⁷⁵⁺ fragment in a cell contacted in vitro with the corrector agent is at least 1-fold, 1.5-fold, 2-fold, 3-fold, 4-fold, 5-fold, 6-fold, 7-fold, 8-fold, 9-fold or 10-fold greater than the amount of accumulation of the same fragment CFTR³⁷⁵⁺ in a cell not contacted with the corrector agent. In some embodiments, the corrector agent action is further characterized in vitro by a similar increase in accumulation of fragment CFTR³⁷³ (or CFTR³⁷⁰) or half-life of fragment CFTR³⁷³ (or CFTR³⁷⁰) in the presence of the corrector compared to such accumulation of fragment CFTR³⁷³ (or CFTR³⁷⁰) or half-life of fragment CFTR³⁷³ (or CFTR³⁷⁰), respectively, in the absence of the corrector. In some embodiments, a maximal accumulation of fragment CFTR³⁸⁰ in a cell expressing said fragment in the presence of a concentration of said corrector agent is achieved at about the same concentration of said corrector agent needed to achieve the maximal accumulation of full-length CFTR in a cell expressing said full-length CFTR. In some embodiments, the amount of accumulation of the CFTR fragment is determined by Western Blot or ELISA. In some embodiments, the corrector agent used in the methods and compositions of the invention does not increase accumulation of a C-form in a fragment CFTR³⁸⁰ containing a mutation or deletion between residues 362-380. In some embodiments, the corrector agent used in the methods and compositions of the invention is capable in vitro of increasing in accumulation of a fragment of the CFTR protein that includes at least the N-terminal 374 amino acids of a polypeptide having a sequence that is at least 80%, 85%, 90%, 91%, 92%, 93%, 94%, 95%, 96%, 97%, 98%, 99% or 100% identical to SEQ ID NO: 1 (i.e., a “fragment CFTR³⁷⁴⁺”) in a cell expressing the fragment in the presence of the corrector as compared to such accumulation of fragment CFTR³⁷⁴⁺ in a cell expressing the fragment in the absence of the corrector.

In some embodiments, the half-life of the fragment CFTR³⁷⁵⁺ is increased in a cell contacted with the corrector agent in vitro as compared to the half-life of the fragment CFTR³⁷⁵⁺ in a cell not contacted with the corrector agent. In some embodiments, the fragment CFTR³⁷⁵⁺ is a fragment CFTR³⁷⁵, fragment CFTR³⁸⁰, fragment CFTR³⁹⁰, fragment CFTR⁴⁰⁰, fragment CFTR⁴¹⁰, fragment CFTR⁴²⁰, fragment CFTR⁴³⁰ or fragment CFTR⁶⁵³.

In some embodiments, the half-life of the fragment CFTR³⁷⁵⁺ in a cell contacted with the corrector agent is at least 1-fold, 1.5-fold, 2-fold, 3-fold, 4-fold, 5-fold, 6-fold, 7-fold, 8-fold, 9-fold or 10-fold as compared to the half-life of the same CFTR³⁷⁵⁺ fragment in a cell not contacted with the corrector agent. In some embodiments, similar increases in half-life values for fragments CFTR³⁸⁰, CFTR⁴³⁰, and/or CFTR⁶⁵³ are observed in a cell expressing the fragment CFTR³⁸⁰, CFTR⁴³⁰, and/or CFTR⁶⁵³ in the presence of the corrector as compared to such half-life for fragments CFTR³⁸⁰, CFTR⁴³⁰, and/or CFTR⁶⁵³ in a cell expressing the fragment CFTR³⁸⁰, CFTR⁴³⁰, and/or CFTR⁶⁵³ in the absence of the corrector.

In some embodiments, proteolysis of fragment CFTR³⁷⁵⁺ by trypsin in the presence of the corrector produces an increased quantity of a 22 kD protease resistant fragment. In some embodiments, the fragment CFTR³⁷⁵⁺ is a fragment CFTR³⁷⁵ or fragment CFTR³⁸⁰. In some embodiments, the corrector agent is capable of increasing the amount of a protease resistant 22 kD fragment produced by proteolysis ΔF508 CFTR in the presence of the corrector.

In some embodiments, the corrector agent used in the methods and compositions of the invention acts through at least one amino acid residue selected from an amino acid residue corresponding to amino acid residues 362-380 of CFTR (SEQ ID NO: 1). In some embodiments, the corrector agent acts through at least one amino acid residue selected from an amino acid residue corresponding to amino acid residues 371-375 of CFTR (SEQ ID NO: 1). In some embodiments, the corrector agent acts through at least one amino acid residue selected from an amino acid residue corresponding to amino acid residues 375-380 of CFTR (SEQ ID NO: 1).

In some embodiments, the corrector agent used in the methods and compositions of the invention is incapable in vitro of increasing the amount of accumulation of a NBD1 fragment (e.g., amino acids 389-678 of SEQ ID NO: 1), a ΔF508 NBD1 fragment, or a fragment of the CFTR protein that includes no more than the N-terminal 373 amino acids of SEQ ID NO: 1 (i.e., a “fragment CFTR³⁷³⁻”). In some embodiments, the amount of accumulation of the NBD1 fragment, ΔF508 NBD1 fragment, or the fragment CFTR³⁷³⁻ in a cell contacted in vitro with the corrector agent is increased no more than 50%, 40%, 30%, 20%, 10%, 5%, 1%, 0.5%, or 0.1% or not at all as compared to the amount of accumulation of the same NBD1 fragment, ΔF508 NBD1 fragment, or fragment CFTR³⁷³⁻ in a cell not contacted with the corrector agent. In some embodiments, the half-life of the NBD1 fragment, ΔF508 NBD1 fragment, or fragment CFTR³⁷³⁻ is not increased or is minimally increased in a cell contacted with the corrector agent in vitro as compared to the half-life of the NBD1 fragment, ΔF508 NBD1 fragment, or fragment CFTR³⁷³⁻ in a cell not contacted with the corrector agent. In some embodiments, the half-life of the NBD1 fragment, ΔF508 NBD1 fragment, or fragment CFTR³⁷³⁻ in a cell contacted with the corrector agent is increased no more than 50%, 40%, 30%, 20%, 10%, 5%, 1%, 0.5%, or 0.1% or not at all as compared to the half-life of the same NBD1 fragment, ΔF508 NBD1 fragment, or fragment CFTR³⁷³⁻ in a cell not contacted with the corrector agent. In some embodiments, the amount of the CFTR fragment is determined by Western Blot or ELISA.

In some embodiments, the corrector agent selectively binds to or interacts with MSD1 of CFTR protein. In some embodiments, the corrector agent is capable of binding to or interacting with MSD1 prior to the synthesis of NBD1. In some embodiments, the corrector agent does not bind to or interact with NBD1, R, MSD2, or NBD2. In some embodiments, the corrector agent does not bind to or interact with a “fragment CFTR³⁷³” (i.e., a fragment consisting of the N-terminal 373 amino acid residues of the full length CFTR protein). In some embodiments, the corrector agent does not bind to or interact with a fragment CFTR³⁷⁰. In some embodiments, the corrector agent is incapable of binding to or interacting with any of an ion channel other than CFTR, an ABC transporter other than CFTR, a misfolded protein other than mutant CFTR, a G-protein coupled receptor, a kinase, a molecular chaperone, an ER stress marker and activation marker. In some embodiments, the corrector agent is incapable of binding to or interacting with any of the following proteins: misfolded P-glycoprotein (e.g., a misfolded P-glycoprotein having a G268V mutation), a misfolded human ERG protein (e.g., a misfolded human ERG protein having a G601S mutation), a misfolded α1-ATZ protein, a misfolded β-glucosidase (e.g., a misfolded β-glucosidase having an N370S mutation), wildtype P-glycoprotein, multidrug resistance 1 (MDR1), multidrug resistance protein 1 (MRP1), MRP2, wildtype human ERG, beta-epithelial sodium channel (β-ENaC), chloride channel 2 (CLC2), K(Ca) ion channel, glutamate receptor 1 (GLuR1), CD25, CD69, CD80, CD83, CD86, CD40, CD40L, CD56, CD152, CD107a, adenosine A2a Receptor, calnexin (CANX), heat shock protein 90 kDa beta (Grp94), Valosin-containing protein, human DnaJ2 protein (Hdj-2 or DNAJA1), Ezrin (VIL2), syntaxin 1A (STX1A), Arf, N+/H+ exchanger, Regulatory Factor 2, PDZK1, Grp78/BiP (KDEL), heat shock protein 70 (Hsp70), activating transcription factor 6 (ATF6), C/EBP-homologous protein/growth arrest and DNA damage-inducible gene 153 (CHOP/GADD153) and protein kinase A (PKA).

In some embodiments, the corrector agent used in the methods and compositions of the invention is capable of improving folding efficiency of MSD1 of CFTR protein. In some embodiments, the corrector agent used in the methods and compositions of the invention is capable of improving folding efficiency of nascent MSD1 of CFTR protein (i.e., a nascent CFTR translation intermediate). In some embodiments, the corrector agent is capable of improving folding efficiency of MSD1 of CFTR protein as MSD1 is being synthesized by a ribosome. In some embodiments, the corrector agent is capable of improving folding efficiency of MSD1 of CFTR protein after MSD1 has been synthesized by the ribosome but before the full-length CFTR protein has been synthesized.

In some embodiments, the corrector agent used in the methods and compositions of the invention is capable of facilitating folding of the CFTR protein. In some embodiments, the corrector agent used in the methods and compositions of the invention is capable of facilitating folding of a mutant CFTR protein such that the mutant CFTR protein in the presence of the corrector agent has a tertiary structure more similar to the tertiary structure of a wildtype CFTR protein than to the tertiary structure of a mutant CFTR protein. In some embodiments, the facilitation of the folding of the mutant CFTR protein is assessed by, e.g., X-ray crystallography, thermal stability assays, aggregation assays, and or FRET based assays.

In some embodiments, the corrector agent used in the methods and compositions of the invention is capable of promoting interaction between MSD1 and NBD1. In some embodiments, the corrector agent is capable of improving the duration or strength of interaction between MSD1 and NBD1 of a nascent or full-length CFTR protein. In some embodiments, the corrector agent is capable of improving the duration or strength of interaction between MSD1 and NBD1 during the biosynthesis of the CFTR protein. In some embodiments, the corrector agent is capable of improving the duration or strength of interaction between ICL1 and NBD1. In some embodiments, the interaction between MSD1 and NBD1 of a mutant CFTR protein in the presence of a corrector agent is more similar to the interaction between MSD1 and NBD1 of a wildtype CFTR protein than to the interaction between MSD1 and NBD1 of the mutant CFTR protein in the absence of the corrector agent. In some embodiments, the corrector agent is capable of improving the duration or strength of interaction between ICL2 and NBD2.

In some embodiments, the characteristics of the corrector agent are determined by using an in vitro assay. In other embodiments, the characteristics of the corrector agent are determined by using an in vivo assay.

In some embodiments, the corrector agent used in the methods and compositions of the invention is capable of increasing ATPase activity of a CFTR protein. In some embodiments, the ATPase activity of a CFTR protein is increased in a cell contacted with the corrector agent in vitro as compared to the ATPase activity of a CFTR protein in a cell not contacted with the corrector agent. While CFTR's predominant function is to operate as an anion channel, it also demonstrates enzymatic activity through hydrolysis of ATP. CFTR has a slow turnover rate for its ATPase activity, as it is only needed to regulate the open/closed state in support of channel function. Measuring the ATP-ase activity may be done in order to determine whether the protein is in a functional conformation. Representative ATP-ase assays are routinely done in the art. See, e.g., Wellhauser et al., Mol Pharmacol, 2009. 75(6): 1430-8.

In some embodiments, the corrector agent used in the methods and compositions of the invention is capable of acting through MSD1 of a CFTR protein fragment lacking the MSD2 domain. In some embodiments, the corrector agent for use in the methods and compositions of the invention is unable to act through CFTR fragments lacking MSD1. In some embodiments, the corrector agent does not act through NBD1, NBD2, R and/or MSD2 during biosynthesis of CFTR. In some embodiments, the corrector agent has no effect on a CFTR protein having mutations in the NBD2, R and/or MSD2 domains.

In some embodiments, the corrector agent used in the methods and compositions of the invention is capable of acting through MSD1 during biosynthesis of a mutant CFTR protein having one or more mutations in MSD1. In some embodiments, the one or more mutations in MSD1 are in the TM1, TM2, TM3, TM4, TM5 or TM6 regions, or any combination thereof. In some embodiments, the mutation is at an amino acid position corresponding to any one of, or combination of, amino acid residues 56, 67, 92, 126, 130, 132, 137, 138, 139, 140, 141, 145, 146, 165, 166, 170, 175, 177, 178, 179, 206, 232, 241, 243, 244, 248, 258, 277, 279, 281, 285, 287, 353, 355, 356, 357, 360, 361, 364, 365, 360, 373, 375, 378, 379, 383, 388, 392, or 394 of SEQ ID NO: 1. In some embodiments, in addition to the mutations in MSD1, the mutant CFTR protein further comprises a mutation at a position corresponding to 508 of SEQ ID NO: 1. In some embodiments the mutation at a position corresponding to 508 of SEQ ID NO: 1 is ΔF508. In some embodiments, the mutation is selected from the group consisting of a substitution of lysine or leucine for glutamic acid at amino acid residue 56 of SEQ ID NO: 1. In some embodiments, the mutation is the substitution of leucine for proline at amino acid residue 67 of SEQ ID NO: 1. In some embodiments, the mutation is selected from the group consisting of a substitution of lysine, glutamine, arginine, valine or aspartic acid for glutamic acid at amino acid residue 92 of SEQ ID NO: 1. In some embodiments, the mutation is the substitution of aspartic acid for glycine at amino acid residue 126 of SEQ ID NO: 1. In some embodiments, the mutation is a substitution of valine for leucine at amino acid residue 130 of SEQ ID NO: 1. In some embodiments, the mutation is a substitution of methionine for isoleucine at amino acid 132 of SEQ ID NO: 1. In some embodiments, the mutation is a substitution of histidine, proline or arginine for a leucine at amino acid residue 137 of SEQ ID NO: 1. In some embodiments, the mutation is the insertion of a leucine at amino acid residue 138 of SEQ ID NO: 1. In some embodiments, the mutation is a substitution of leucine or arginine for histidine at amino acid residue 139 of SEQ ID NO: 1. In some embodiments, the mutation is a substitution of serine or leucine for proline at amino acid residue 140 of SEQ ID NO: 1. In some embodiments, the mutation is a substitution of aspartic acid for alanine at amino acid residue 141 of SEQ ID NO: 1. In some embodiments, the mutation is a substitution of histidine for leucine at amino acid residue 145 of SEQ ID NO: 1. In some embodiments, the mutation is a substitution of arginine for histidine at amino acid residue 146 of SEQ ID NO: 1. In some embodiments, the mutation is a substitution of serine for leucine at amino acid residue 165 of SEQ ID NO: 1. In some embodiments, the mutation is a substitution of glutamine for lysine at amino acid residue 166 of SEQ ID NO: 1. In some embodiments, the mutation is a substitution of cysteine, glycine, or histidine for arginine at amino acid residue 170 of SEQ ID NO: 1. In some embodiments, the mutation is a substitution of valine for isoleucine at amino acid residue 175 of SEQ ID NO: 1. In some embodiments, the mutation is a substitution of threonine for isoleucine at amino acid residue 177 of SEQ ID NO: 1. In some embodiments, the mutation is a substitution of glutamic acid or arginine for glycine at amino acid residue 178 of SEQ ID NO: 1. In some embodiments, the mutation is a substitution of lysine for glutamine at amino acid residue 179 of SEQ ID NO: 1. In some embodiments, the mutation is a substitution of tryptophan for leucine at amino acid residue 206 of SEQ ID NO:1. In some embodiments, the mutation is the substitution of aspartic acid for valine at amino acid residue 232 of SEQ ID NO: 1. In some embodiments, the mutation is a substitution of arginine for glycine at amino acid residue 241 of SEQ ID NO: 1. In some embodiments, the mutation is a substitution of leucine for methionine at amino acid residue 243 of SEQ ID NO: 1. In some embodiments, the mutation is a substitution of lysine for methionine at amino acid residue 244 of SEQ ID NO: 1. In some embodiments, the mutation is a substitution of threonine for arginine at amino acid residue 248 of SEQ ID NO: 1. In some embodiments, the mutation is a substitution of glycine for arginine at amino acid residue 258 of SEQ ID NO: 1. In some embodiments, the mutation is a substitution of arginine for tryptophan at amino acid residue 277 of SEQ ID NO: 1. In some embodiments, the mutation is a substitution of aspartic acid for glutamic acid at amino acid residue 279 of SEQ ID NO: 1. In some embodiments, the mutation is a substitution of threonine for methionine at amino acid residue 281 of SEQ ID NO: 1. In some embodiments, the mutation is a substitution of phenylalanine for isoleucine at amino acid residue 285 of SEQ ID NO: 1. In some embodiments, the mutation is a substitution of tyrosine for asparagine at amino acid residue 287 of SEQ ID NO: 1. In some embodiments, the mutation is a substitution of lysine for isoleucine at amino acid residue 336 of SEQ ID NO: 1. In some embodiments, the mutation is a substitution of histidine for glutamine at amino acid residue 353 of SEQ ID NO: 1. In some embodiments, the mutation is a substitution of serine for proline at amino acid residue 355 of SEQ ID NO: 1. In some embodiments, the mutation is a substitution of serine for tryptophan at amino acid residue 356 of SEQ ID NO: 1. In some embodiments, the mutation is a substitution of lysine or arginine for glutamine at amino acid residue 359 of SEQ ID NO: 1. In some embodiments, the mutation is a substitution of lysine or arginine for threonine at amino acid residue 360 of SEQ ID NO: 1. In some embodiments, the mutation is a substitution of arginine for tryptophan at amino acid residue 361 of SEQ ID NO: 1. In some embodiments, the mutation is a substitution of serine for proline at amino acid residue 364 of SEQ ID NO: 1. In some embodiments, the mutation is a substitution of leucine for proline at amino acid residue 365 of SEQ ID NO: 1. In some embodiments, the mutation is the insertion of aspartic acid and lysine after amino acid residue 370 of SEQ ID NO: 1. In some embodiments, the mutation is a substitution of glutamic acid for aspartic acid at amino acid residue 373 of SEQ ID NO: 1. In some embodiments, the mutation is a substitution of phenylalanine for leucine at amino acid residue 375 of SEQ ID NO: 1. In some embodiments, the mutation is a substitution of arginine for glutamine at amino acid residue 378 of SEQ ID NO: 1. In some embodiments, the mutation is a substitution of lysine for glutamic acid at amino acid residue 379 of SEQ ID NO: 1. In some embodiments, the mutation is a substitution of serine for leucine at amino acid residue 383 of SEQ ID NO: 1. In some embodiments, the mutation is a substitution of methionine for threonine at amino acid residue 388 of SEQ ID NO: 1. In some embodiments, the mutation is a substitution of alanine or glycine for valine at amino acid residue 392 of SEQ ID NO: 1. In some embodiments, the mutation is a substitution of arginine for methionine at amino acid residue 394 of SEQ ID NO: 1.

In some embodiments, the corrector agent useful in the methods of the invention is capable of acting through MSD1 during biosynthesis of a CFTR protein having a mutation in a region other than MSD1. In some embodiments, the mutation is in the NBD1, NBD2, MSD2, R, ICL1, ICL2, ICL3, ICL4 or N- or C-terminal regions of the CFTR protein. In some embodiments, the mutation is in the coupling helix extending from transmembrane 2 (TM2) region or transmembrane 3 (TM3) region of the CFTR protein. In some embodiments, the mutation is at an amino acid position corresponding to amino acid residue 149 or 192 of SEQ ID NO: 1. In some embodiments, the mutation is in the NBD1 domain of CFTR protein. In some embodiments, the mutation in NBD1 is a deletion of phenylalanine at amino acid residue 508 of SEQ ID NO: 1. In some embodiments, the mutant CFTR protein may have any combination of mutations in MSD1, NBD1, NBD2, MSD2, R, ICL1, ICL2, ICL3, ICL4 or N- or C-terminal regions of the CFTR protein described herein. In some embodiments, the mutation is the substation of glutamic acid for alanine at amino acid residue 455 of SEQ ID NO: 1. In some embodiments, the mutation is the substitution of aspartic acid for histidine at amino acid residue 1054 of SEQ ID NO: 1. In some embodiments, the mutation is the substitution of arginine for glycine at amino acid residue 1061 of SEQ ID NO: 1. In some embodiments, the mutation is the substitution of histidine for arginine at amino acid residue 1066 of SEQ ID NO: 1. In some embodiments, the mutation is the substitution of leucine for phenylalanine at amino acid residue 1074 of SEQ ID NO: 1. In some embodiments, the mutation is the substitution of arginine for histidine at amino acid residue 1085 of SEQ ID NO: 1.

In some embodiments, the corrector agent binds or interacts with nascent MSD1 during biosynthesis of CFTR. In other embodiments, the corrector agent binds to or interacts with MSD1 in a CFTR lacking MSD2. In other embodiments, the corrector agent binds to or interacts with MSD1 in a CFTR lacking MSD2, NBD1 and NBD2. In other embodiments, the corrector agent binds to or interacts with MSD1 in a CFTR lacking MSD2 and NBD2. In some embodiments, the corrector agent interacts with or binds to the CFTR protein during the CFTR's biosynthesis in the ER of a cell. The skilled worker is aware of numerous assays routinely used to determine how a compound, e.g., a corrector agent, binds a target region of a protein, e.g., a specific region of CFTR. For example, once a corrector agent is identified, its binding site may be identified by utilizing routine procedures such as crystallography and/or protein fragment analysis. In certain embodiments, the corrector agent can be chosen on the basis of its selectivity for the CFTR protein, or for a specific region of the CFTR protein (e.g., the MSD1 domain). In other embodiments, the corrector agent can be chosen on the basis of its selectivity for a specific CFTR mutant over another specific CFTR mutant.

In some embodiments, the corrector agent has an ED₅₀ of 1 mM or less, more preferably of 1 μM or less, and even more preferably of 1 nM or less.

In some embodiments, the corrector agent has a molecular weight less than 2500 amu, more preferably less than 1500 amu, and even more preferably less than 750 amu.

In some embodiments, the corrector agent is a small molecule, provided that the small molecule is not a proteasome inhibitor and any of the compounds disclosed in Table 1.

In some embodiments, the corrector agent is a nucleic acid molecule. In some embodiments, the nucleic acid molecule is made up of deoxyribonucleotides, ribonucleotides, modified nucleotides, or any combinations thereof. In some embodiments, the nucleic acid molecule is in a plasmid. In some embodiments, the nucleic acid molecule is delivered in a liposome or a nanoparticle formulation. In some embodiments, the nucleic acid molecule is delivered in a viral vector.

In some embodiments, the corrector agent is a polypeptide, i.e., a “polypeptide corrector agent.” The polypeptide corrector agents described herein may be identified or characterized using any one of, or combination of, the assays described herein. In particular embodiments, the polypeptide corrector agent interacts or binds with the MSD1 domain of a CFTR protein in a cell. In some embodiments, the polypeptide corrector agent is at least 3, 5, 10, 15, 20, 25, 30, 35, 40, 45, 50, 55, 60, 65, 70, 75, 80, 90, 100, 110, 120, 130, 140, 150, 175, 200, 225 or 250 amino acids in length. In some embodiments, the polypeptide corrector agent is 5-10, 5-25, 5-50, 5-75, 5-100, 5-150 or 5-200 amino acids in length. In some embodiments, a polypeptide corrector agent is membrane permeable.

In certain aspects, a polypeptide corrector agent comprises a chimeric polypeptide which further comprises one or more fusion domains. These fusion domains may be used, for example, to purify the polypeptide corrector agent. Well known examples of such fusion domains include, but are not limited to, polyhistidine, Glu-Glu, glutathione S transferase (GST), thioredoxin, protein A, protein G, and an immunoglobulin heavy chain constant region (Fc), maltose binding protein (MBP), which are particularly useful for isolation of the fusion proteins by affinity chromatography. For the purpose of affinity purification, relevant matrices for affinity chromatography, such as glutathione-, amylase-, and nickel- or cobalt-conjugated resins are used. Fusion domains also include “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the polypeptide corrector agents therefrom. The liberated polypeptide corrector agents can then be isolated from the fusion domain by subsequent chromatographic separation.

In some embodiments, the corrector agent comprises a chimeric polypeptide comprising a first portion that is a polypeptide corrector agent, and a second portion that serves as a targeting moiety. A “targeting moiety” is any compound moiety (e.g., a polypeptide, a polynucleotide, a small molecule) that is capable of targeting a tissue or tissues affected in a subject. In some embodiments, the targeting moiety targets a subject's lungs, pancreas, liver, intestines, sinuses, and/or sex organs. In some embodiments, the targeting moiety may be a single chain Fv (scFv) portion of an antibody that targets, e.g., lung tissue, in a subject. In some embodiments, the targeting moiety targets an intracellular compartment, e.g., the ER. In some embodiments, the targeting moiety portion of a chimeric polypeptide is capable of transporting a corrector agent portion to a particular organ, tissue, cell type or intracellular component in a CF patient.

In some embodiments, the corrector agent comprises a chimeric polypeptide that comprises a first portion that is a polypeptide corrector agent, and a second portion that serves as an internalizing moiety. An “internalizing moiety” is any moiety that facilitates the internalization of the corrector agent into a cell. In some embodiments, the internalizing moiety is a TAT-polypeptide, which is capable of transporting a fused polypeptide portion across a cell membrane and to the ER. See, e.g., Kim et al., 2012, PLoS One, 12(e51813): 1-14.

In some embodiments, a polypeptide corrector agent may be a fusion protein with all or a portion of an Fc region of an immunoglobulin. The Fc region, or portion of the Fc region, may serve as either a targeting moiety and/or an internalizing moiety. As is known, each immunoglobulin heavy chain constant region comprises four or five domains. The domains are named sequentially as follows: CH1-hinge-CH2-CH3(-CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE. As used herein, the term, “immunoglobulin Fc region” refers to the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In some embodiments the immunoglobulin Fc region comprises at least an immunoglobulin hinge region of a CH2 domain and a CH3 domain, and preferably lacks the CH1 domain. In some embodiments, the class of immunoglobulin from which the heavy chain constant region is derived is IgG (Igγ) (γ subclasses 1, 2, 3, or 4). Other classes of immunoglobulin, IgA (Igα), IgD (Igδ), IgE (Igε) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fc γ or the homologous domains in any of IgA, IgD, IgE, or IgM. Furthermore, it is contemplated that substitution or deletion of amino acids within the immunoglobulin heavy chain constant regions may be useful in the practice of the disclosure. For example, amino acid substitutions may be introduced in the upper CH2 region to create a Fc variant with reduced affinity for Fc receptors (Cole et al. (1997) J. IMMUNOL. 159:3613). One of ordinary skill in the art can prepare such constructs using well known molecular biology techniques.

In certain embodiments, a polypeptide corrector agent may further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptide corrector agents may contain non-amino acid elements, such as lipids, poly- or mono-saccharide, and phosphates. Effects of such non-amino acid elements on the functionality of a polypeptide corrector agent may be tested for its biological activity, for example, its ability to act through MSD1 of a CFTR protein during the biosynthesis of the CFTR protein.

In some embodiments, a polypeptide corrector agent may be modified with nonproteinaceous polymers. In some embodiments, the polymer is polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337. PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161).

In some embodiments, the polypeptide corrector agent may contain one or more modifications that are capable of stabilizing the polypeptides. For example, such modifications enhance the in vitro half life of the polypeptides, enhance circulatory half life of the polypeptides or reduce proteolytic degradation of the polypeptides.

In some embodiments, the corrector agent is an antibody, antibody-fragment, or antibody-like protein that binds to CFTR in order to act through MSD1 during the biosynthesis of CFTR protein. In some embodiments, the corrector agent is an antibody, antibody-fragment, or antibody-like protein that binds to the MSD1 domain of the CFTR protein, the C-terminal region or the N-terminal region of the CFTR protein. In some embodiments, the corrector agent is an antibody fragment.

In some embodiments, the corrector agent is a humanized antibody, antibody-fragment, or antibody-like protein that binds to CFTR in order to act through MSD1 during the biosynthesis of CFTR protein. “Humanized” refers to an immunoglobulin such as an antibody, wherein the amino acids directly involved in antigen binding, the so-called complementary determining regions (CDR), of the heavy and light chains are not of human origin, while the rest of the immunoglobulin molecule, the so-called framework regions of the variable heavy and light chains, and the constant regions of the heavy and light chains are modified so that they correspondence of more closely correspond to human sequences. Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species.

The corrector agents described herein may be identified or characterized using any one of, or combination of, the assays described herein.

### B. Methods of Treating Cystic Fibrosis Subjects

In one aspect, the invention relates to a method of treating a subject having CF with a corrector agent described herein or a pharmaceutical composition comprising a corrector agent described herein. The corrector agents described herein are for use in treating a subject having CF. A method of treating a CF subject, as defined herein, comprises the administration of a corrector agent or a pharmaceutically acceptable composition comprising a corrector agent to a CF subject. The population of subjects treated by the method of treatment includes subjects suffering from the undesirable condition or disease, as well as subjects at risk for development of the condition or disease. In some embodiments, the CF subject is administered an “effective dose” or “effective amount” of any of the corrector agents described herein. In some embodiments, the corrector agent is a small molecule, a polypeptide, a peptidomimetic, an antibody, an antibody fragment, an antibody-like protein, or a nucleic acid.

In some embodiments, a corrector agent is capable of improving lung function in a CF subject. Improved lung function may be measured by various assays routinely used in the art. For example, improved lung function may be assessed by measuring any improvement in Forced Vital Capacity (FVC). FVC is the volume of air that can forcibly be blown out after full inspiration, measured in liters. In addition, improved lung function may be assessed by measuring any improvement Forced Expiratory Volume in 1 second (FEV1). FEV1 is the volume of air that can forcibly be blown out in one second, after full inspiration. A further test for improved lung function is measuring the FEV1/FVC ratio. In some embodiments, the corrector agent is capable of improving pancreatic function in a CF subject.

In some embodiments, the method of the invention comprises treating a CF subject having misfolded CFTR protein. In some embodiments, the misfolded CFTR protein misfolds as a result of a mutation in the gene encoding the CFTR protein. In some embodiments, the misfolded CFTR protein misfolds as a result of one or more mutations in the CFTR protein's MSD1 domain. In some embodiments, the one or more mutations in the MSD1 domain is in the TM1, TM2, TM3, TM4, TM5 or TM6 regions or any combination thereof. In some embodiments, the one or more mutations is at an amino acid position corresponding to any one of, or combination of, amino acid residues 92, 126, 130, 132, 137, 138, 139, 140, 141, 145, 146, 165, 166, 170, 175, 177, 178, 179, 206, 241, 243, 244, 248, 258, 277, 279, 281, 285, 287, 353, 355, 356, 357, 360, 361, 364, 365, 360, 373, 375, 378, 379, 383, 388, 392, or 394 of SEQ ID NO: 1. In some embodiments, in addition to the mutations in MSD1, the mutant CFTR protein further comprises a mutation at a position corresponding to 508 of SEQ ID NO: 1. In some embodiments the mutation at a position corresponding to 508 of SEQ ID NO: 1 is ΔF508. In some embodiments, the mutation is selected from the group consisting of a substitution of lysine or leucine for glutamic acid at amino acid residue 56 of SEQ ID NO: 1. In some embodiments, the mutation is the substitution of leucine for proline at amino acid residue 67 of SEQ ID NO: 1. In some embodiments, the mutation is selected from the group consisting of a substitution of lysine, glutamine, arginine, valine or aspartic acid for glutamic acid at amino acid residue 92 of SEQ ID NO: 1. In some embodiments, the mutation is the substitution of an aspartic acid for glycine at amino acid residue 126 of SEQ ID NO: 1. In some embodiments, the mutation is a substitution of valine for leucine at amino acid residue 130 of SEQ ID NO: 1. In some embodiments, the mutation is a substitution of methionine for isoleucine at amino acid 132 of SEQ ID NO: 1. In some embodiments, the mutation is a substitution of histidine, proline or arginine for leucine at amino acid 137 residue of SEQ ID NO: 1. In some embodiments, the mutation is an insertion of leucine at amino acid residue 138 of SEQ ID NO: 1. In some embodiments, the mutation is a substitution of leucine or arginine for histidine at amino acid residue 139 of SEQ ID NO: 1. In some embodiments, the mutation is a substitution of serine or leucine for proline at amino acid residue 140 of SEQ ID NO: 1. In some embodiments, the mutation is a substitution of aspartic acid for alanine at amino acid residue 141 of SEQ ID NO: 1. In some embodiments, the mutation is a substitution of histidine for leucine at amino acid residue 145 of SEQ ID NO: 1. In some embodiments, the mutation is a substitution of arginine for histidine at amino acid residue 146 of SEQ ID NO: 1. In some embodiments, the mutation is a substitution of serine for leucine at amino acid residue 165 of SEQ ID NO: 1. In some embodiments, the mutation is a substitution of glutamine for lysine at amino acid residue 166 of SEQ ID NO: 1. In some embodiments, the mutation is a substitution of cysteine, glycine, or histidine for arginine at amino acid residue 170 of SEQ ID NO: 1. In some embodiments, the mutation is a substitution of valine for isoleucine at amino acid residue 175 of SEQ ID NO: 1. In some embodiments, the mutation is a substitution of threonine for isoleucine at amino acid residue 177 of SEQ ID NO: 1. In some embodiments, the mutation is a substitution of glutamic acid or arginine for glycine at amino acid residue 178 of SEQ ID NO: 1. In some embodiments, the mutation is a substitution of lysine for glutamine at amino acid residue 179 of SEQ ID NO: 1. In some embodiments, the mutation is a substitution of tryptophan for leucine at amino acid residue 206 of SEQ ID NO:1. IN some embodiments, the mutation is a substitution of aspartic acid for valine at amino acid residue 232 of SEQ ID NO: 1. In some embodiments, the mutation is a substitution of arginine for glycine at amino acid residue 241 of SEQ ID NO: 1. In some embodiments, the mutation is a substitution of leucine for methionine at amino acid residue 243 of SEQ ID NO: 1. In some embodiments, the mutation is a substitution of lysine for methionine at amino acid residue 244 of SEQ ID NO: 1. In some embodiments, the mutation is a substitution of threonine for arginine at amino acid residue 248 of SEQ ID NO: 1. In some embodiments, the mutation is a substitution of glycine for arginine at amino acid residue 258 of SEQ ID NO: 1. In some embodiments, the mutation is a substitution of arginine for tryptophan at amino acid residue 277 of SEQ ID NO: 1. In some embodiments, the mutation is a substitution of aspartic acid for glutamic acid at amino acid residue 279 of SEQ ID NO: 1. In some embodiments, the mutation is a substitution of threonine for methionine at amino acid residue 281 of SEQ ID NO: 1. In some embodiments, the mutation is a substitution of phenylalanine for isoleucine at amino acid residue 285 of SEQ ID NO: 1. In some embodiments, the mutation is a substitution of tyrosine for asparagine at amino acid residue 287 of SEQ ID NO: 1. In some embodiments, the mutation is a substitution of lysine for isoleucine at amino acid residue 336 of SEQ ID NO: 1. In some embodiments, the mutation is a substitution of histidine for glutamine at amino acid residue 353 of SEQ ID NO: 1. In some embodiments, the mutation is a substitution of serine for proline at amino acid residue 355 of SEQ ID NO: 1. In some embodiments, the mutation is a substitution of serine for tryptophan at amino acid residue 356 of SEQ ID NO: 1. In some embodiments, the mutation is a substitution of lysine or arginine for glutamine at amino acid residue 359 of SEQ ID NO: 1. In some embodiments, the mutation is a substitution of lysine or arginine for threonine at amino acid residue 360 of SEQ ID NO: 1. In some embodiments, the mutation is a substitution of arginine for tryptophan at amino acid residue 361 of SEQ ID NO: 1. In some embodiments, the mutation is a substitution of serine for proline at amino acid residue 364 of SEQ ID NO: 1. In some embodiments, the mutation is a substitution of leucine for proline at amino acid residue 365 of SEQ ID NO: 1. In some embodiments, the mutation is the insertion of aspartic acid and lysine after amino acid residue 370 of SEQ ID NO: 1. In some embodiments, the mutation is a substitution of glutamic acid for aspartic acid at amino acid residue 373 of SEQ ID NO: 1. In some embodiments, the mutation is a substitution of phenylalanine for leucine at amino acid residue 375 of SEQ ID NO: 1. In some embodiments, the mutation is a substitution of arginine for glutamine at amino acid residue 378 of SEQ ID NO: 1. In some embodiments, the mutation is a substitution of lysine for glutamic acid at amino acid residue 379 of SEQ ID NO: 1. In some embodiments, the mutation is a substitution of serine for leucine at amino acid residue 383 of SEQ ID NO: 1. In some embodiments, the mutation is a substitution of methionine for threonine at amino acid residue 388 of SEQ ID NO: 1. In some embodiments, the mutation is a substitution of alanine or glycine for valine at amino acid residue 392 of SEQ ID NO: 1. In some embodiments, the mutation is a substitution of arginine for methionine at amino acid residue 394 of SEQ ID NO: 1.

In some embodiments, the method of the invention comprises treating a CF subject having a CFTR mutation in a region other than MSD1. In some embodiments, the mutation is in the NBD1, NBD2, MSD2, R, ICL1, ICL2, ICL3, ICL4 or N- or C-terminal regions of the CFTR protein. In some embodiments, the mutation is in the coupling helix extending from transmembrane 2 (TM2) region or transmembrane 3 (TM3) region of the CFTR protein. In some embodiments, the mutation is at an amino acid position corresponding to amino acid residue 149 or 192 of SEQ ID NO: 1. In some embodiments, the subject has a mutation in the NBD1 domain of CFTR protein. In some embodiments, the mutation in NBD1 is a deletion of phenylalanine at amino acid residue 508 of SEQ ID NO: 1. In some embodiments, the mutant CFTR protein may have any combination of mutations in MSD1, NBD1, NBD2, MSD2, R, ICL1, ICL2, ICL3, ICL4 or N- or C-terminal regions of the CFTR protein described herein. In some embodiments, the mutation is the substation of glutamic acid for alanine at amino acid residue 455 of SEQ ID NO: 1. In some embodiments, the mutation is the substitution of aspartic acid for histidine at amino acid residue 1054 of SEQ ID NO: 1. In some embodiments, the mutation is the substitution of arginine for glycine at amino acid residue 1061 of SEQ ID NO: 1. In some embodiments, the mutation is the substitution of histidine for arginine at amino acid residue 1066 of SEQ ID NO: 1. In some embodiments, the mutation is the substitution of leucine for phenylalanine at amino acid residue 1074 of SEQ ID NO: 1. In some embodiments, the mutation is the substitution of arginine for histidine at amino acid residue 1085 of SEQ ID NO: 1.

In some embodiments, the method comprises administering a corrector agent to a subject having a mutant CFTR protein that is sensitive to potentiation by ivacaftor. Ivacaftor potentiation sensitivity of a particular mutant CFTR protein may be determined by administering various concentrations of candidate corrector agents to a cell culture monolayer, wherein the cells in the culture are expressing a specific mutant CFTR, and then utilizing an Ussing chamber (MUsE; Vertex Pharmaceuticals Inc.) to record the transepithelial current in the culture. Specifically, forskolin (which elicits CFTR chloride channel currents) is added to the culture in the presence or absence of ivacaftor, and if the ivacaftor potentiates the forskolin induced CFTR chloride channel currents, then the mutant CFTR protein expressed by the cells in the culture is sensitive to potentiation by ivacaftor. See, e.g., Example 8.

### C. Combination Therapies

In some embodiments, the method comprises administering to a CF subject a corrector agent and at least one additional therapeutic agent. In some embodiments, the additional therapeutic agent is a bronchodilator, an antibiotic, a mucolytic agent, a nutritional agent or an agent that blocks ubiquitin-mediated proteolysis.

A bronchodilator for use as an additional therapeutic agent may be a short-acting β2 agonist, a long-acting β2 agonist or an anticholinergic. In some embodiments, the bronchodilator is any one of, or combination of, salbutamol/albuterol, levosalbutamol/levalbuterol, pirbuterol, epinephrine, ephedrine, terbutaline, salmeterol, clenbuterol, formoterol, bambuterol, indacaterol, theophylline, tiotropium or ipratropium bromide.

An antibiotic for use as an additional therapeutic agent may be any antibiotic chosen by a physician for reducing lung infections in a CF subject. In some embodiments, the antibiotic is any one of, or combination of, xicillin, clavulanate potassium, aztreonam, ceftazidime, ciprofloxacin, gentamicin or tobramycin.

A mucolytic agent for use as an additional therapeutic agent may be any agent used for breaking down the gel structure of mucus and therefore decreasing its elasticity and viscosity. In some embodiments, the mucolytic agent is N-acetylcysteine, dornase alpha, hypertonic solution, mannitol, gelsolin or thymosin-β4.

A nutritional agent for use as an additional therapeutic agent may be any agent that may be used to promote adequate growth and weight gain in a CF subject. In some embodiments, the nutritional agent is any one of, or combination of, vitamins A, D, E, or K, sodium chloride, calcium, or pancreatic enzymes. In some embodiments, the nutritional agent is a multivitamin. In some embodiments, the nutritional agent is a high calorie food or food supplement.

An agent that blocks ubiquitin-mediated proteolysis for use as an additional therapeutic agent is any agent that blocks proteasomal degradation of misfolded CFTR. In some embodiments, the agent that blocks ubiquitin-mediated proteolysis is a proteasome inhibitor. In some embodiments, the agent that blocks ubiquitin-mediated proteolysis is selected from the group consisting of a peptide aldehyde, a peptide boronate, a peptide α′β′-epoxyketone, a peptide ketoaldehyde or a β-lactone. In some embodiments, the agent that blocks ubiquitin-mediated proteolysis is selected from the group consisting of bortezomib, carfilzomib, marizomib, CEP-18770, MLN-9708 and ONX-0912. In some embodiments, the method comprises administering to a CF subject any of the corrector agents described herein and at least one additional therapeutic agent, wherein the at least one additional therapeutic agent is at least one additional corrector agent. In some embodiments, the invention provides a formulation or pharmaceutical preparation comprising any of the corrector agents described herein and at least additional therapeutic agent, wherein the at least one additional therapeutic agent is at least one additional corrector agent. In some embodiments, the at least one additional corrector agent also acts on the MSD1 domain. In other embodiments, the at least one additional corrector agent acts on a domain other than the MSD1 domain, e.g., NBD1, MSD2, NBD2 and/or the R domains or any of the regions linking these domains.

In some embodiments, the corrector agent and the at least one additional therapeutic agent are administered to a CF subject concurrently. In some embodiments, the corrector agent and the at least one additional therapeutic agent are administered to a CF subject consecutively. In some embodiments, the corrector agent and the at least one additional therapeutic agent are administered via the same route of administration. In some embodiments, the corrector agent and the at least one additional therapeutic agent are administered on different dosing schedules and/or via different routes of administration. In some embodiments, the first dose of a corrector agent is administered to a CF subject at a point after the administration to the subject of at least a first dose of the at least one additional therapeutic agent. In other embodiments, the first dose of the at least one additional therapeutic agent is administered to a CF subject at a point after the administration to the subject of at least a first dose of a corrector agent.

### D. Screening for and Identifying Corrector Agents

In one aspect, the invention provides a method of screening for and/or identifying a corrector agent. A “test agent,” as used herein, is an agent (e.g., a small molecule, polypeptide, peptidomimetic, antibody, antibody fragment, antibody-like protein, or nucleic acid) used in any of the screening assays described below for the purposes of determining if the agent is a candidate corrector agent. A “candidate corrector agent” is an agent, e.g., a small molecule, polypeptide, peptidomimetic, antibody, antibody fragment, antibody-like protein, or nucleic acid, that has not yet been confirmed to be a corrector agent, but that has at least one characteristic consistent with a corrector agent, e.g., increases accumulation and/or half-life of CFTR³⁷⁵⁺ fragments, increases amount of mature CFTR protein in a cell, does not affect ubiquitination machinery in a cell, increases trafficking of mutant CFTR from the ER, increases chloride transport, improves channel gating of a CFTR protein, increases ATPase activity of a CFTR protein, and increases resistance of a CFTR to proteolytic degradation. A candidate corrector agent is confirmed to be a corrector agent if it is determined to be a candidate corrector agent in at least 1, 2, 3, 4, 5, 6, 7 or 8 of the different assays described below. In some embodiments, a candidate corrector agent is a corrector agent if it is confirmed that the candidate corrector agent: a) increases resistance of CFTR to proteolytic degradation, b) increases chloride transport or improves channel gating of a CFTR protein, c) increases trafficking of CFTR from the ER or increases the amount of mature CFTR protein in a cell, and d) increases accumulation and or half-life of CFTR³⁷⁵⁺ fragments.

Numerous assays are available for screening and identifying a corrector agent. A wide range of techniques are known in the art for screening agents (e.g., polypeptides or small molecules) to determine if the test agents have a desired property. For example, screening techniques are well known for screening gene products of combinatorial libraries made by point mutations and truncations, and, for that matter, for screening cDNA libraries for gene products having a certain property. The most widely used techniques for screening large gene libraries typically comprise cloning the gene library into replicable expression vectors, transforming appropriate cells with the resulting library of vectors, and expressing the combinatorial genes under conditions in which detection of a desired activity (e.g., acting through MSD1 of CFTR protein during the biosynthesis of the CFTR protein) facilitates relatively easy isolation of the vector encoding the gene whose product was detected.

Each of the illustrative assays described below are amenable to rapid screening or high through-put analysis as necessary to screen large numbers of degenerate sequences created by combinatorial mutagenesis techniques. The assays described below are likewise amenable to rapid screening or high through-put analysis of other types of agents, e.g., small molecules.

i. Effects on MSD1 CFTR Fragments

In certain embodiments, the method assesses the amount of accumulation of a CFTR fragment in a cell. In some embodiments, the CFTR fragment is a CFTR³⁷³⁻ fragment or a CFTR³⁷⁵⁺ fragment. In some embodiments, the method comprises the steps of: a) contacting a test agent with a cell expressing a CFTR³⁷⁵⁺ fragment, b) measuring the amount of the CFTR³⁷⁵⁺ fragment in the cell, and c) comparing the amount of the CFTR³⁷⁵⁺ fragment in the cell with the amount of the CFTR³⁷⁵⁺ fragment in a cell not contacted with the test agent, wherein if the amount of the CFTR³⁷⁵⁺ fragment in cell contacted with the test agent is greater than the amount of CFTR³⁷⁵⁺ fragment in cell not contacted with the test agent, the test agent is a candidate corrector agent. In certain embodiments, the method of screening for a candidate corrector agent comprises the steps of: a) administering a test agent to a subject expressing a CFTR³⁷⁵⁺ fragment, b) measuring the amount of the CFTR³⁷⁵⁺ fragment in the subject, and c) comparing the amount of the CFTR³⁷⁵⁺ fragment in the subject with the amount of the CFTR³⁷⁵⁺ fragment in a subject not administered the test agent, wherein if the amount of the CFTR³⁷⁵⁺ fragment in cell of the subject administered the test agent is greater than the amount of CFTR³⁷⁵⁺ fragment in cell of the subject not administered the test agent, the test agent is a candidate corrector agent. In certain embodiments, the method of screening for a candidate corrector agent comprises the steps of: a) contacting a test agent with a cell expressing a CFTR³⁷³⁻ fragment, b) measuring the amount of the CFTR³⁷³⁻ fragment in the cell, and c) comparing the amount of the CFTR³⁷³⁻ fragment in the cell with the amount of the CFTR³⁷³⁻ fragment in a cell not contacted with the test agent, wherein if the amount of the CFTR³⁷³⁻ fragment in cell contacted with the test agent is greater than the amount of the CFTR³⁷³⁻ fragment in cell not contacted with the test agent, the test agent is a candidate corrector agent. In certain embodiments, the method of screening for a candidate corrector agent comprises the steps of: a) administering a test agent to a subject expressing a CFTR³⁷³⁻ fragment, b) measuring the amount of the CFTR³⁷³⁻ fragment in the subject, and c) comparing the amount of the CFTR³⁷³⁻ fragment in the subject with the amount of the CFTR³⁷³⁻ fragment in a subject not administered the test agent, wherein if the amount of the CFTR³⁷³⁻ fragment in cell of the subject administered the test agent is greater than the amount of CFTR³⁷³⁻ fragment in cell of the subject not administered the test agent, the test agent is a candidate corrector agent. In some embodiments, the CFTR³⁷⁵⁺ fragment is a CFTR³⁷⁵ fragment or CFTR³⁸⁰ fragment. In some embodiments, the CFTR³⁷³⁻ fragment is a CFTR³⁷³ fragment or a CFTR³⁷⁰ fragment. In some embodiments, the amount of CFTR protein fragments are measured in a subject by measuring the amount of CFTR protein fragment in a sample taken from a subject. In some embodiments, the CFTR protein fragment is a fragment that does not include the NBD1, R, NDB2, or MSD2 domains. In some embodiments, the CFTR protein fragment is the result of a CFTR gene mutation that results in a truncated CFTR protein. In some embodiments, the CFTR gene mutation is a mutation associated with causing CF in human subjects. In some embodiments, in place of the CFTR³⁷³⁻ fragments, the method tests accumulation of a CFTR protein fragment that does not comprise the MSD1 domain, but that comprises the NBD1, R, NBD2 and/or MSD2 domains. In some embodiments, the amount of accumulation of the CFTR protein in the cells or the subject are determined by utilizing Western Blot or ELISA analysis. See, e.g., Example 1. In some embodiments, the candidate corrector agent is an agent that, upon administration to a subject expressing a CFTR³⁷⁵⁺ fragment or upon contacting a cell expressing a CFTR³⁷⁵⁺ fragment increases accumulation of the CFTR³⁷⁵ fragment such that the amount of the CFTR³⁷⁵ fragment are at least 50%, 75%, 100%, 200%, 300%, 400%, 500%, 600%, 700%, 800%, 900%, or 1000% greater than the amount of the CFTR³⁷⁵ fragment in the subject or cell prior to administration, or in the absence, of the candidate corrector agent. In some embodiments, the candidate corrector agent is an agent that, upon administration to a subject, or prior to contacting with a cell, expressing a CFTR³⁷⁵ fragment increases CFTR³⁷⁵ fragment accumulation such that the amount of CFTR³⁷⁵ fragment in the subject or cell is at least 2.5%, 5%, 7.5%, 10%, 12.5%, 15%, 17.5%, 20%, 22.5%, 25%, 30%, 35%, 40%, 45%, 50%, 55%, 60%, 65%, 70%, 75%, 80%, 85%, 90%, 95% and 100% of the amount of CFTR³⁷⁵⁺ fragment observed in a healthy control subject or healthy control cell. In some embodiments, the control cell or cell not contacted with the test agent is the same type of cell as the cell treated with the corrector agent.

ii. Increasing the Amount of Mature Mutant CFTR Protein

In some embodiments, the corrector agent useful in the methods of the invention is capable of increasing the amount of mature CFTR protein (e.g., mutant CFTR protein) in a cell or a subject. In certain embodiments, the method of screening for a candidate corrector agent comprises the steps of: a) contacting a test agent with a cell expressing a CFTR protein, b) measuring the amount of mature CFTR protein in the cell, and c) comparing the amount of mature CFTR protein in the cell with the amount of the CFTR protein fragment in a cell not contacted with the test agent, wherein if the amount of the mature CFTR protein in cell contacted with the test agent is greater than the amount of mature CFTR protein in cell not contacted with the test agent, the test agent is a candidate corrector agent. In certain embodiments, the method of screening for a candidate corrector agent comprises the steps of: a) administering a test agent to a subject expressing a CFTR protein, b) measuring the amount of mature CFTR protein in the subject, and c) comparing the amount of mature CFTR protein in the subject with the amount of the CFTR protein fragment in a subject not administered the test agent, wherein if the amount of the mature CFTR protein in cell of the subject administered the test agent is greater than the amount of mature CFTR protein in cell of the subject not administered the test agent, the test agent is a candidate corrector agent. In some embodiments, the amount of mature CFTR protein is measured in a subject by measuring the amount of mature CFTR protein in a sample taken from a subject.

Similar to other integral membrane glycoproteins, the initial stages of CFTR biosynthesis begin with the formation in the endoplasmic reticulum (ER) membrane of a core-glycosylated 135- to 140-kDa “immature” form that is a precursor to the “mature” 150- to 160-kDa CFTR that contains complex, endoH-resistant oligosaccharide chains (Kopito, R R, 1999, Physiol Rev, 79(1): S167-S173). As used herein, the term “mature CFTR” refers to CFTR that migrates as a diffuse, 150- to 160-kDa band that is resistant to digestion with endoH and thus presumed to have matured at least to the cis/medial cisternae of the Golgi apparatus. The term “immature CFTR” refers to the 135- to 140-kDa, endoH-sensitive form corresponding to nascent CFTR that has not been processed by mannosidases in the cis/medial Golgi. As such, the amount of mature CFTR in a cell or in a subject may be determined by performing a routine assay, such as a Western Blot, in order to determine the molecular weight of the CFTR protein present in the cell or sample from the subject. See, e.g., Example 2.

In some embodiments, the candidate corrector agent is an agent that, upon administration to a subject or upon contacting a cell having a CFTR protein (e.g., a mutant CFTR protein), result in an amount of the mature CFTR protein that is at least 50%, 75%, 100%, 200%, 300%, 400%, 500%, 600%, 700%, 800%, 900%, or 1000% greater than the amount of CFTR protein in the subject or cell prior to, or in the absence of, administration of the candidate corrector agent. In some embodiments, the candidate corrector agent is an agent that, upon administration to a subject or upon contacting a cell having a mutant CFTR protein, results in an amount of CFTR protein in the subject or cell that is at least 2.5%, 5%, 7.5%, 10%, 12.5%, 15%, 17.5%, 20%, 22.5%, 25%, 30%, 35%, 40%, 45%, 50%, 55%, 60%, 65%, 70%, 75%, 80%, 85%, 90%, 95% and 100% of the amount of CFTR protein observed in a healthy control subject or healthy control cell.

An alternative method for determining whether a test agent is capable of increasing the amount of mature CFTR protein in a cell or subject is to assess the amount of CFTR present at the cell surface of cells treated with/without the test agent. As immature CFTR is typically unable to be transported to the cell surface, any CFTR present at the cell surface of a cell is presumed to be “mature.” Cell surface CFTR may be isolated by a variety of means well-known in the art, including for example, the commercial Pierce Cell Surface Protein Isolation Kit (Thermo Fisher Scientific, Rockford, Ill.). Following isolation of membrane containing cell surface CFTR, the amount of the cell surface CFTR in the membrane may be assessed by using an anti-CFTR antibody and assays such as, but not limited to, Western Blot or ELISA. Alternatively, cell surface CFTR amounts may be assessed by immunocytochemistry or immunohistochemistry.

In some embodiments, the control cell or cell not contacted with the test agent is the same type of cell as the cell treated with the corrector agent.

iii. Effects on Ubiquitination Machinery

The candidate corrector agents disclosed herein alter the ubiquitination amount and/or pattern of mutant CFTR in a cell. In some embodiments, the candidate corrector agents disclosed herein alter the ubiquitination amount and/or pattern of mutant CFTR in a subject. In certain embodiments, the method of screening for a candidate corrector agent comprises the steps of: a) contacting a test agent with a cell expressing a mutant CFTR protein, b) measuring the amount or pattern of ubiquitination of the mutant CFTR protein in the cell, and c) comparing the amount or pattern of ubiquitination of the mutant CFTR protein in the cell with the ubiquitination pattern or amount of the mutant CFTR protein in a cell not contacted with the test agent, wherein if the amount or pattern of ubiquitination of the mutant CFTR protein in the cell contacted with the test agent are different than the amount or patterns of mutant CFTR protein in the cell not contacted with the test agent, the test agent is a candidate corrector agent. In certain embodiments, the method of screening for a candidate corrector agent comprises the steps of: a) administering a test agent to a subject expressing a mutant CFTR protein, b) measuring the amount or pattern of ubiquitination of the mutant CFTR protein in the subject, and c) comparing the amount or pattern of ubiquitination of the mutant CFTR protein in the subject with the ubiquitination pattern or amount of the mutant CFTR protein in a subject not administered the test agent, wherein if the amount or pattern of ubiquitination of the mutant CFTR protein in the subject administered the test agent is different than amount or pattern of ubiquitination of the of the mutant CFTR protein in a subject not administered the test agent, the test agent is a candidate corrector agent. In some embodiments, the CFTR ubiquitination pattern or amount are measured in a subject by measuring the CFTR ubiquitination pattern or amount in a sample from a subject. To determine whether a test agent affects the ubiquitination pattern of mutant CFTR in a cell or a subject, any one of numerous routine ubiquitination assays may be utilized. For example, TUBE (Tandem Ubiquitin Binding Entity) affinity resin may be used to purify polyubiquitinated proteins from a cell or sample from a subject that has been treated with or without an agent, and then the amount of ubiquitinated proteins can be assessed. See, e.g., Example 4. If lower amount of ubiquitinated mutant CFTR are present in a sample treated with a candidate corrector agent, then the test agent is a candidate corrector agent. In some embodiments, the control cell or cell not contacted with the test agent is the same type of cell as the cell treated with the corrector agent.

iv. Effects on Endoplasmic Reticulum Trafficking

In some embodiments, the candidate corrector agents disclosed herein are capable of increasing trafficking of a CFTR protein (e.g., mutant CFTR protein) out of the ER (i.e., increasing ER export). In certain embodiments, the method of screening for a candidate corrector agent comprises the steps of: a) contacting a test agent with a cell expressing a CFTR protein, b) measuring the ER export of the CFTR protein in the cell, and c) comparing the ER export of the CFTR protein in the cell with the ER export of the CFTR protein in a cell not contacted with the test agent, wherein if the ER export of the CFTR protein in the cell contacted with the test agent is greater than the ER export of the CFTR protein in the cell not contacted with the test agent is a candidate corrector agent. In certain embodiments, the method of screening for a candidate corrector agent comprises the steps of: a) administering a test agent to a subject expressing a CFTR protein, b) measuring the ER export of the CFTR protein in a cell from the subject, and c) comparing the ER export of the CFTR protein in the cell from the subject with the ER export of the CFTR protein in a cell from a subject not administered the test agent, wherein if the ER export of the CFTR protein in the subject administered the test agent is greater than the ER export of the CFTR protein in a subject not administered the test agent, the test agent is a candidate corrector agent. In some embodiments, the ER export of CFTR is measured in a subject by measuring the ER export of CFTR from a sample taken from a subject. The effects of a test agent on ER export of CFTR may be assessed, for example, by utilizing a CFTR metabolic pulse-chase analysis. In such a pulse-chase analysis, cells expressing wildtype or mutant CFTR are treated with, or without, the test agent in the presence or absence of the ER-transport blocker, brefeldin A. At various intervals following treatment with the test agent, cells are harvested and the total amount of immature CFTR is assessed. See, e.g., Example 3. A test agent that induces an increase in the total amount of immature CFTR in a brefeldin A treated cell is a candidate corrector agent. In some embodiments, the control cell or cell not contacted with the test agent is the same type of cell as the cell treated with the corrector agent.

v. Chloride Transport

In some embodiments, the candidate corrector agent is capable of increasing chloride transport of a CFTR (e.g., mutant CFTR protein). In certain embodiments, the method of screening for a candidate corrector agent comprises the steps of: a) contacting a test agent with a cell expressing a CFTR protein, b) measuring the chloride transport of the CFTR protein of the cell, and c) comparing the chloride transport of the CFTR protein of the cell with the chloride transport of the CFTR protein of a cell not contacted with the test agent, wherein if the chloride transport of the CFTR protein in the cell contacted with the test agent is greater than the chloride transport of the CFTR protein in the cell not contacted with the test agent, the test agent is a candidate corrector agent. In certain embodiments, the method of screening for a candidate corrector agent comprises the steps of: a) administering a test agent to a subject expressing a CFTR protein, b) measuring the chloride transport of the CFTR protein in a cell from the subject, and c) comparing the chloride transport of the CFTR protein in the cell from the subject with the chloride transport of the CFTR protein in a cell from a subject not contacted with the test agent, wherein if the chloride transport of the CFTR protein in the subject administered the test agent is greater than the chloride transport of the CFTR protein in a subject not administered the test agent, the test agent is a candidate corrector agent. In some embodiments, chloride transport of CFTR is measured in a subject by measuring chloride transport of CFTR in a sample from a subject. CFTR chloride transport may be determined by utilizing standard assays known in the art, including, but not limited to, the utilization of Ussing chamber recordings. Ussing chamber assays use electrodes to measure ion flow across the membranes of cells grown into a monolayer with tight junctions. See, e.g., Example 5. If the test agent increases ion flow across cell membranes of cells expressing CFTR, the test agent is a candidate corrector agent. In some embodiments, the candidate corrector agent increases ion flow by at least 25%, 50%, 100%, 150%, 200%, 250%, 300%, 350%, 400%, 500%, 600%, 700%, 800%, 900%, or 1000% as compared to control cells that were not treated with the candidate corrector agent. In some embodiments, the control cell or cell not contacted with the test agent is the same type of cell as the cell treated with the corrector agent.

vi. Improvement in Channel Gating

In some embodiments, the corrector agent is capable of improving channel gating of a CFTR protein (e.g., mutant CFTR protein). In certain embodiments, the method of screening for a candidate corrector agent comprises the steps of: a) contacting a test agent with a cell expressing a CFTR protein, b) measuring the CFTR protein channel gating in the cell, and c) comparing the CFTR protein channel gating in the cell with the CFTR protein channel gating in a cell not contacted with the test agent, wherein if the channel gating of the CFTR protein in the cell contacted with the test agent is greater than the channel gating of the CFTR protein in the cell not contacted with the test agent, the test agent is a candidate corrector agent. In certain embodiments, the method of screening for a candidate corrector agent comprises the steps of: a) administering a test agent to a subject expressing a CFTR protein, b) measuring the CFTR protein channel gating in the subject, and c) comparing the CFTR protein channel gating in the subject with the CFTR protein channel gating in a subject not administered the test agent, wherein if the channel gating of the CFTR protein in the subject administered the test agent is greater than the channel gating of the CFTR protein in a subject not administered the test agent, the test agent is a candidate corrector agent. In some embodiments, the CFTR channel gating activity is measured in a subject by measuring the CFTR channel gating activity in a sample from a subject.

As used herein, “improvements in CFTR channel gating” means increasing the open probability of a CFTR channel protein. Improvements in channel gating may be determined by utilizing any one of numerous standard assays known in the art, including, but not limited to, the utilization of single-channel patch-clamp recording assays. Patch clamp recording assays measure the opening and closing rates of single channels, in which patches of the cell membrane are isolated using a micropipette tip and these patches are hooked up to microelectrodes. See, e.g., Example 6 and Devor et al., 2000, Am J Physiol Cell Physiol, 279(2): C461-79 and Dousmanis, et al., 2002, J Gen Physiol, 119(6): 545-59. If the test agent increases the probability of the CFTR protein being open in cells expressing CFTR, the test agent is a candidate corrector agent. In some embodiments, a candidate corrector agent improves channel gating of a CFTR protein by at least 50%, 100%, 150%, 200%, 250%, 300%, 350%, 400%, 500%, 600%, 700%, 800%, 900% or 1000% as compared to a control cell that expressing the CFTR but not treated with the candidate corrector agent. In some embodiments, the control cell or cell not contacted with the test agent is the same type of cell as the cell treated with the corrector agent.

vii. Increasing ATPase Activity

In some embodiments, the corrector agent used in the methods and compositions of the invention is capable of increasing ATPase activity of a CFTR protein (e.g., mutant CFTR protein). In certain embodiments, the method of screening for a candidate corrector agent comprises the steps of: a) contacting a test agent with a cell expressing a CFTR protein, b) measuring the ATPase activity of the CFTR protein in the cell, and c) comparing the ATPase activity of the CFTR protein in the cell with the ATPase activity of the CFTR protein in a cell not contacted with the test agent, wherein if the ATPase activity of the CFTR protein in the cell contacted with the test agent is greater than the ATPase activity of the CFTR protein in the cell not contacted with the test agent, the test agent is a candidate corrector agent. In certain embodiments, the method of screening for a candidate corrector agent comprises the steps of: a) administering a test agent to a subject expressing a CFTR protein, b) measuring the ATPase activity of the CFTR protein in the subject, and c) comparing the ATPase activity of the CFTR protein in the subject with the ATPase activity of the CFTR protein in a subject not administered the test agent, wherein if the ATPase activity of the CFTR protein in the subject administered the test agent is greater than the ATPase activity of the CFTR protein in a subject not administered the test agent, the test agent is a candidate corrector agent. In some embodiments, the CFTR ATPase activity levels are measured in a subject by measuring the ATPase activity levels in a sample from a subject. While CFTR's predominant function is to operate as an anion channel, it also demonstrates enzymatic activity through hydrolysis of ATP. CFTR has a slow turnover rate for its ATPase activity, as it is only needed to regulate the open/closed state in support of channel function. Measuring the ATP-ase activity may be done in order to determine whether the protein is in a functional conformation. Representative ATP-ase assays are routinely done in the art. See, e.g., Wellhauser et al., Mol Pharmacol, 2009. 75(6): 1430-8. In some embodiments, the control cell or cell not contacted with the test agent is the same type of cell as the cell treated with the corrector agent.

viii. Increasing Resistance to Proteolytic Degradation

In some embodiments, the corrector agent is capable of increasing resistance of CFTR protein (e.g., mutant CFTR protein or a CFTR³⁷⁵⁺ fragment) to proteolytic degradation. It has previously been demonstrated that ΔF508 CFTR is more susceptible than wildtype CFTR to proteolytic digestion by proteases such as trypsin. Without being bound by theory, the increased proteolytic sensitivity of ΔF508 CFTR protein may be attributed to an unfolded or partially folded conformation of the ΔF508 CFTR protein in which portions of the polypeptide are exposed to proteases that are otherwise protected from proteases in a more compact wildtype CFTR conformation.

In some embodiments, the corrector agent used in the methods and compositions of the invention is capable of increasing resistance to proteolysis, i.e., reducing proteolysis, of a mutant CFTR protein or a CFTR³⁷⁵⁺ fragment. In certain embodiments, the method of screening for a candidate corrector agent comprises the steps of: a) contacting a test agent with a cell expressing a mutant CFTR protein or a CFTR³⁷⁵⁺ fragment, b) measuring the amount of proteolytic degradation of the mutant CFTR protein or the CFTR³⁷⁵⁺ fragment in the cell, and c) comparing the amount of proteolytic degradation of the mutant CFTR protein or the CFTR³⁷⁵⁺ fragment in the cell with the amount of proteolytic degradation of the mutant CFTR protein or CFTR³⁷⁵⁺ fragment in a cell not contacted with the test agent, wherein if the amount of proteolytic degradation of the mutant CFTR protein or the CFTR³⁷⁵⁺ fragment in the cell contacted with the test agent is greater than the amount of proteolytic degradation of the mutant CFTR protein or the CFTR³⁷⁵⁺ fragment in the cell not contacted with the test agent, the test agent is a candidate corrector agent. In certain embodiments, the method of screening for a candidate corrector agent comprises the steps of: a) administering a test agent to a subject expressing a mutant CFTR protein, b) measuring the amount of proteolytic degradation of the mutant CFTR protein in the subject, and c) comparing the amount of proteolytic degradation of the mutant CFTR protein in the subject with the amount of proteolytic degradation of the mutant CFTR protein in a subject not administered the test agent, wherein if the amount of proteolytic degradation of the mutant CFTR protein in the subject administered the test agent is greater than the amount of proteolytic degradation of the mutant CFTR protein in a subject not administered the test agent, the test agent is a candidate corrector agent. In some embodiments, the amount of proteolytic degradation of CFTR is measured in a subject by measuring the amount of proteolytic degradation of CFTR in a sample from a subject.

In some embodiments, protease resistance/sensitivity is measured by using a proteolysis assay. Such assays are known in the art. In some embodiments, the protease resistance is determined by assessing the amount of proteolytically degraded mutant CFTR or CFTR³⁷⁵⁺ fragment in the presence of carboxypeptidase, trypsin, V8 protease, papain or chymotrypsin and in the presence or absence of a test agent. In some embodiments, the amount of proteolytically degraded mutant CFTR or CFTR³⁷⁵⁺ fragment is determined by Western Blot.

In some embodiments, the candidate corrector agent increases protease resistance of the full-length CFTR protein (e.g., a full-length mutant CFTR protein). In other embodiments, the candidate corrector agent increases protease resistance of a fragment of the full-length CFTR protein. In some embodiments, the fragment of full-length CFTR protein is a fragment comprising at least MSD1. In some embodiments, the fragment of full-length CFTR protein is a CFTR³⁷⁵⁺ fragment. In some embodiments, the CFTR³⁷⁵⁺ fragment is a CFTR³⁷⁵ fragment or a CFTR³⁸⁰ fragment. In some embodiments, a candidate corrector agent increases protease resistance (i.e., reduces protease sensitivity) of a mutant CFTR protein or a CFTR³⁷⁵⁺ fragment by at least 50%, 100%, 150%, 200%, 250%, 300%, 350%, 400%, 500%, 600%, 700%, 800%, 900% or 1000% as compared to a control cell that expressing the mutant CFTR or CFTR³⁷⁵⁺ fragment but not treated with the candidate corrector agent. In some embodiments, the control cell or cell not contacted with the test agent is the same type of cell as the cell treated with the corrector agent.

### E. Corrector Agent Compositions

In another aspect, the invention relates to pharmaceutical compositions comprising any of the corrector agents, described herein, and a pharmaceutically acceptable carrier, adjuvant or vehicle. In certain embodiments, these compositions optionally further comprises one or more additional therapeutic agents.

It will also be appreciated that certain of the corrector agents for use in the present methods can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a subject in need is capable of providing, directly or indirectly, a corrector agent as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a corrector agent of this invention that, upon administration to a recipient, is capable of providing, either directly or indirectly, a corrector agent of this invention or an active metabolite or residue thereof. Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Pharmaceutically acceptable salts of the corrector agents of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. This invention also envisions the quaternization of any basic nitrogen-containing groups of the corrector agents disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the corrector agents of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The amount of corrector agent administered to a subject will vary from subject to subject, depending on the species, age, and general condition of the subject, the severity of CF, the particular agent, its mode of administration, and the like. The corrector agents described herein are preferably formulated in dosage unit form for ease of administration and uniformity of dosage. The expression “dosage unit form” as used herein refers to a physically discrete unit of corrector agent appropriate for the subject to be treated. It will be understood, however, that the total daily usage of the corrector agents and compositions described herein will be decided by the attending physician within the scope of sound medical judgment. The specific effective dose for any particular subject or organism will depend upon a variety of factors including the type of CF being treated (e.g., the mutation causing the CF), the severity of the CF; the activity of the specific corrector agent being employed; the specific composition employed; the age, body weight, general health, sex and diet of the subject; the time of administration, route of administration, and rate of excretion of the specific corrector agent employed; the duration of the treatment; drugs used in combination or coincidental with the specific corrector agent employed, and like factors well known in the medical arts.

The pharmaceutically acceptable compositions of this invention can be administered to humans and other animals using any route of administration effective for treating CF, improving CF, improving the symptoms of CF, lessening the severity of CF or lessening the severity of the symptoms of CF. The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like. In certain embodiments, the corrector agents of the invention may be administered orally or parenterally at dosage amounts of about 0.01 mg/kg to about 50 mg/kg and, in some embodiments, from about 1 mg/kg to about 25 mg/kg, of subject body weight per day, one or more times a day, to obtain the desired therapeutic effect. Effective doses may be extrapolated from dose-response curves derived from in vitro or animal model test systems. Alternatively, the corrector agent is administered once every other day, twice per week, weekly, once every other week or monthly.

Liquid dosage forms for oral administration include, but are not limited to, pharmaceutically acceptable emulsions, microemulsions, solutions, suspensions, syrups and elixirs. In addition to the corrector agents, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of any of the corrector agents described herein, it is often desirable to slow the absorption of the corrector agent from subcutaneous or intramuscular injection. This may be accomplished by the use of a liquid suspension of crystalline or amorphous material with poor water solubility. The rate of absorption of the corrector agent then depends upon its rate of dissolution that, in turn, may depend upon crystal size and crystalline form. Alternatively, delayed absorption of a parenterally administered corrector agent is accomplished by dissolving or suspending the corrector agent in an oil vehicle. Injectable depot forms are made by forming microencapsule matrices of the corrector agent in biodegradable polymers such as polylactide-polyglycolide. Depending upon the ratio of corrector agent to polymer and the nature of the particular polymer employed, the rate of corrector agent release can be controlled. Examples of other biodegradable polymers include poly(orthoesters) and poly(anhydrides). Depot injectable formulations are also prepared by entrapping the corrector agent in liposomes or microemulsions that are compatible with body tissues.

Compositions for rectal or vaginal administration are preferably suppositories which can be prepared by mixing the corrector agents described herein with suitable non-irritating excipients or carriers such as cocoa butter, polyethylene glycol or a suppository wax which are solid at ambient temperature but liquid at body temperature and therefore melt in the rectum or vaginal cavity and release the active corrector agent.

Solid dosage forms for oral administration include capsules, tablets, pills, powders, and granules. In such solid dosage forms, the active corrector agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the corrector agent only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The corrector agents described herein can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the corrector agents may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of any of the corrector agents described herein include ointments, pastes, creams, lotions, gels, powders, solutions, sprays, inhalants or patches. The corrector agent is admixed under sterile conditions with a pharmaceutically acceptable carrier and any needed preservatives or buffers as may be required. The present invention contemplates the use of transdermal patches, which have the added advantage of providing controlled delivery of a corrector agent to the body. Such dosage forms are prepared by dissolving or dispensing the corrector agent in the proper medium. Absorption enhancers can also be used to increase the flux of the corrector agent across the skin. The rate can be controlled by either providing a rate controlling membrane or by dispersing the corrector agent in a polymer matrix or gel.

The corrector agents described herein or pharmaceutically acceptable compositions thereof may also be incorporated into compositions for coating an implantable medical device, such as prostheses, artificial valves, vascular grafts, stents and catheters. Accordingly, the present invention, in another aspect, includes a composition for coating an implantable device comprising any of the corrector agents described herein as described generally above, and in classes and subclasses herein, and a carrier suitable for coating the implantable device. In still another aspect, the present invention includes the use of an implantable device coated with a composition comprising a corrector agent, and a carrier suitable for coating the implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

In certain embodiments, the corrector agents discussed herein, including pharmaceutical preparations, are non-pyrogenic. In other words, in certain embodiments, the compositions are substantially pyrogen free. In one embodiment the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in relatively large dosages and/or over an extended period of time (e.g., such as for the subject's entire life), even small amounts of harmful and dangerous endotoxin could be dangerous. In certain specific embodiments, the endotoxin and pyrogen amounts in the composition are less then 10 EU/mg, or less then 5 EU/mg, or less then 1 EU/mg, or less then 0.1 EU/mg, or less then 0.01 EU/mg, or less then 0.001 EU/mg.

### F. Animal Models of CF

The methods and corrector agents described herein may be tested in any one of several animal models in order to further characterize the corrector agent, or in order to optimize dosing or for the generation of formulations.

At least fourteen different mouse models of CF exist, including mice having null or mutant forms of CFTR. See, e.g., Fisher et al., 2011, Methods Mol Biol, 742:311-34. These mouse models recapitulate various CF-related organ pathologies to varying degrees, and the severity of the phenotypes of these mice are generally based on the amounts of CFTR mRNA present (See, e.g., Fisher et al.). Most of the mouse models display phenotypes such as severe abnormalities of the gastrointestinal tract, failure to thrive, decreased survival and hyperinflammatory responses in the airway (See, e.g., Fisher et al.). These mice also may display defects in cAMP-inducible chloride permeability in the nasal epithelium, decreased mucociliary clearance, reduced fertility, mild pancreatic dysfunction and liver abnormalities (See, e.g., Fisher et al.). However, these mouse models do not display the significant spontaneous lung disease as observed in CF human subjects (See, e.g., Fisher et al.).

Recently, a pig and ferret model of CF have been developed. See, e.g., Keiser, et al., 2011, Curr Opin Pulm Medic, 17: 478-483. These models more closely recapitulate the CF symptoms observed in human subjects. In particular, a pig having a CFTR^{ΔF508/ΔF508} mutation develops lung disease and severe gallbladder disease and displays exocrine pancreatic defects and hepatic lesions. See, e.g., Keiser et al. In some embodiments, the candidate corrector agent is administered to the CFTR^{ΔF508/ΔF508} pig, and effects of the corrector agent on this pig's CF-like symptoms are assessed.

### EXAMPLES

The following examples are included merely to illustrate certain aspects and embodiments of the invention, and are not intended to limit the scope of the invention.

### Example 1

### Fragment Analysis

In order to determine the site of action in CFTR on which a test agent acts, a fragment analysis assay is employed.

CFTR Mutant Construct Transfection:

CFTR constructs representing CF-disease causing point mutants or truncated biogenic intermediates (e.g., the MSD1 domain portion of CFTR) are made in the CFTR-pcDNA3.1(+) plasmid using the QuikChange protocol (Stratagene). HEK293 cells from ATCC are maintained in Dulbecco's Modified Eagle's medium (DMEM, GIBCO) supplemented with 1% fetal bovine serum (Hyclone) and antibiotics (100 units/ml penicillin and 100 μg/ml streptomycin, GIBCO) at 37° C. in an atmosphere of 5% CO₂. Cell transfections are performed using Effectene reagent (Qiagen). The empty pcDNA3.1(+) vector is used to ensure equal microgram quantities of DNA are used in all transfection reactions.

Transfected cells are then left untreated or are treated with varying concentrations of the test agent, a positive control agent (e.g., lumacaftor), or DMSO. The cells are incubated with the test agent for a period of time before the test agent is washed from the cells and the cells are harvested for CFTR maturation analysis.

Western Blot Studies to Monitor CFTR Maturation:

To monitor CFTR maturation, FRT-FlpIn cells stably expressing normal or mutant CFTR forms are harvested in ice-cold Dulbecco's-phosphate buffered saline (without calcium and magnesium) and collected at 1000×g at 4° C. Cell pellets are lysed in 1% NP-40, 0.5% sodium deoxycholate, 200 mM NaCl, 10 mM Tris, pH 7.8 and 1 mM EDTA plus protease inhibitor cocktail (1:250, Roche) for 30 minutes on ice. Nuclei and insoluble material are removed by centrifugation at 10,000×g for 10 minutes at 4° C. to yield cleared lysate. Approximately 12 μg total protein of cleared lysate is heated in Laemmli buffer with 5% β-mercaptoethanol at 37° C. for 5 minutes and subjected to electrophoretic separation on a 3-8% Tris-Acetate gel (Invitrogen), transferred to nitrocellulose and probed for either CFTR protein using monoclonal CFTR antibody 769 (J. Riordan, University of North Carolina) or GAPDH, the gel loading control, using a polyclonal antibody to GAPDH (Santa Cruz Biotech). CFTR and GAPDH are visualized by infrared fluorescence detection (Odyssey IRDye 800, goat anti-mouse secondary) using the Li-Cor, and quantified using Odyssey Analysis Software.

If cells treated with a test agent produce more of a given CFTR fragment (e.g., a CFTR³⁷⁵⁺ fragment such as a CFTR³⁷⁵ fragment or a CFTR³⁸⁰ fragment) than cells treated with a control agent, than this is indicative that the test agent is a candidate corrector agent. If cells treated with a test corrector agent produce more of a given CFTR fragment (e.g., a CFTR³⁷⁵⁺ fragment such as a CFTR³⁷⁵ fragment or a CFTR³⁸⁰ fragment) than cells treated with the positive control agent (e.g., lumacaftor), than this is indicative that a test agent superior to the positive control agent has been identified.

### Example 2

### Profiling Corrector Affinity Using Various CFTR Mutants

In order to measure the EC₅₀ for a test agent against a panel of CFTR mutations, several assays are utilized.

Cell Lines:

To generate a host cell line to express different mutant CFTR forms, a single integration site is introduced into FRT cells (Michael Welsh, University of Iowa, Iowa City, Iowa) by transfecting a construct containing the Flp Recombination Target site (pFRT/lacZeo, Invitrogen, Carlsbad, Calif.). To select stably transfected clones containing pFRT/lacZeo, the cells are grown under 500 μg/ml Zeocin selection in growth media containing Coon's modified Ham's F12, 10% FBS, 1% Pen/Strep, 0.23% Na-Bicarbonate. The clone with the most transcriptionally active genomic locus is selected based on expression of β-galactosidase, which is encoded by the lacZ gene. Single site integration is confirmed by Southern blot.

The normal CFTR coding region is cloned into the pcDNA5/Flp recombination target site vector (Invitrogen, Carlsbad, Calif.) between EcoRV and ApaI sites. The normal CFTR clone (Johanna Rommens, Sick Kids Hospital, Toronto) is obtained from a non-CF subject with a polymorphism at amino acid 1475 (V1475M) compared to the published normal CFTR sequence. QuickChange XL site-directed mutagenesis kit (Stratagene, Cambridge, UK) is used to introduce different CFTR gene mutations into the normal CFTR coding sequence, and each mutation is confirmed by sequencing the CFTR coding, 5′-untranslated, and 3′-untranslated regions.

Cell lines expressing either normal or a single mutant CFTR form are generated by co-transfecting the CFTR cDNA and the Flp recombinase expressed from the plasmid, pOG44 (Invitrogen, Carlsbad, Calif.) into the FlpIn FRT host cell line generated as described above. Transfected cells are selected by growth in the presence of 200 μg/ml hygromycin-B. Surviving cells are pooled and expanded at 37° C. in Coon's modified Ham's F12 containing 10% FBS, 1% Pen/Strep, 0.23% Na-Bicarbonate containing 200 μg/ml hygromycin-B.

Transfected cells are then left untreated or are treated with varying concentrations of the test agent, a positive control agent (e.g., lumacaftor), or DMSO. The cells are incubated with the test agent for a period of time before the agent is washed from the cells and the cells are harvested for RNA analysis or CFTR maturation analysis.

RNA Analysis:

Total RNA is isolated from the treated and untreated cells using RNeasy (Qiagen) and post-treated with DNase I (Ambion, Valencia, Calif.). RNA quantity and quality are assessed by spectrophotometry using a Nanodrop 1000 (Thermo Scientific). Real-time PCR assays are performed using an Applied Biosystems 7900HT sequence detector (Applied Biosystems, Foster City, Calif.). Briefly, 1 μg of total RNA is reverse-transcribed to cDNA using the High-Capacity cDNA RT Kit (Applied Biosystems), according to the manufacturer's instructions. Each amplification mixture (20 μl) contained 25 ng of reverse-transcribed RNA, 8 μM forward primer, 8 μM reverse primer, 2 μM dual-labeled fluorogenic probe (Applied Biosystems), and 10 μl of 2× Taqman Universal PCR Master Mix (Applied Biosystem). Primers and probes are from Applied Biosystems. For human CFTR, the forward primer is 5′-CATTGCAGTGGGCTGTAAACTC-3′, the reverse primer is 5′-CTTCTGTTGGCATGTCAATGAACTT-3′, and the probe is 6FAM-AGATCGCATCAAGCTATC-3′. For rat ribosomal protein L32 (RPL32), the forward primer is 5′-GAGTAACAAGAAAACCAAGCACATG-3′, the reverse primer is 5′-TTGACATTGTGG ACCAGAAACTTC-3′, and the probe is 6FAM-CCTAGCGGCTTCC-3′. PCR thermocycling parameters are 50° C. for 2 min, 95° C. for 10 min, and 40 cycles of 92° C. for 15 s and 60° C. for 1 min. All samples are run in triplicate and normalized to RPL32 run in the same well. Results are expressed as the cycle threshold (Ct) at which the amplified CFTR product is first detected normalized to the Ct of RPL32.

Ussing Chamber Recordings to Monitor CFTR Activity:

Ussing chamber studies are used to measure the forskolin-stimulated short circuit current (I_{T}) in recombinant FRT-Flp-In cells expressing CFTR. Cells grown on Costar® Snapwell™ cell culture inserts are mounted in an Ussing chamber (Physiologic Instruments, Inc., San Diego, Calif.), and the I_{T} is measured in the presence of a basolateral to apical chloride gradient using a voltage-clamp system (Department of Bioengineering, University of Iowa, IA). The basolateral solution contains (in mM) 145 NaCl, 0.83 K₂HPO₄, 3.3 KH₂PO₄, 1.2 MgCl₂, 1.2 CaCl₂, 10 Glucose, 10 HEPES (pH 7.35, NaOH) and the apical solution contained (in mM) 145 NaGluconate, 1.2 MgCl₂, 1.2 CaCl₂, 10 glucose, 10 HEPES (pH 7.35, NaOH). The basolateral membrane is permeabilized with 260 μg/mL nystatin 30 min prior to recording.

To activate CFTR, the adenylate cyclase activator, forskolin (10 μM), is added to the bath to increase the intracellular amounts of cAMP. The forskolin-stimulated I_{T} is abolished by CFTR inhibitors and is absent in FRT cells not expressing CFTR, indicating that the measured current is CFTR-mediated chloride transport. The forskolin-stimulated I_{T} is normalized to the mean forskolin-stimulated I_{T} measured from 4 separate FRT cell lines expressing a normal CFTR (204.5±29.9 μA/cm2) and expressed as % normal CFTR chloride transport. The forskolin-stimulated I_{T} in the absence of Ivacaftor is reported as the baseline level of CFTR-mediated chloride.

If cells treated with a test agent produce more active mutant CFTR protein than cells treated with a negative control agent, than this is indicative that the test agent is a candidate corrector agent. If cells treated with a test agent produce more active mutant CFTR protein than cells treated with the positive control agent (e.g., lumacaftor), than this is indicative that a candidate corrector agent superior to the positive control agent has been identified. Briefly, EC₅₀ of the test agent is determined by applying increasing amounts of the test agent to the transfected cells and then measuring the amounts of mutant CFTR activity for each dose until saturation is reached, i.e., the point at which increasing the concentration of the test agent does not increase the level of mutant CFTR activity achieved.

Western Blot Studies to Monitor CFTR Maturation:

Western Blots were performed as described in Example 1. To quantify CFTR maturation, the relative amount of CFTR protein is normalized to GAPDH measured in the identical protein sample, and these amounts are used for subsequent calculations. CFTR maturation is expressed as a ratio of mature to total (mature plus immature) CFTR forms and as a percentage of the mature form of normal CFTR. CFTR processing is considered to be normal if it is within 3 SD of the mean level of maturation for normal CFTR measured in 5 separate FRT cell lines expressing normal CFTR (0.9±0.04; mean±SD; n=5). The CFTR processing defect is considered to be severe if the ratio of mature to total CFTR was within 3 SD of the mean level for ΔF508-CFTR (0.09±0.05; mean±SD; n=3).

If cells treated with a test agent produce more mature mutant CFTR protein than cells treated with a negative control agent, then this is indicative that the test agent is a corrector agent. If cells treated with a test agent produce more mutant CFTR protein than cells treated with the positive control agent (e.g., lumacaftor), then this is indicative that a candidate corrector agent superior to the positive control agent has been identified. Briefly, EC₅₀ of the test agent is determined by applying increasing amounts of the test agent to the transfected cells and then measuring the mutant CFTR protein amounts for each dose until saturation is reached, i.e., the point at which increasing the concentration of the candidate corrector agent does not increase the total amount of mature mutant CFTR protein amounts achieved.

### Example 3

### ER Export

In order to assess the effects of a test agent on ER export of mutant CFTR, an assay is performed in which mutant CFTR is trapped in the ER by brefeldin A in the presence or absence of the test agent.

CFTR Metabolic Pulse-Chase Analysis:

HEK-293 cells expressing CFTR or ΔF508-CFTR are incubated for 16 hours in assay media (HyQ CCM5 with 1% heat-inactivated FBS) with DMSO, a positive control agent (e.g., lumacaftor) or test agent. For metabolic labeling, cells are starved for 30 min in DMEM without cysteine and methionine with 1% dialyzed FBS in the presence of the candidate corrector agent. Cells are then pulsed with [³⁵S] methionine and cysteine EXPRESS35 label (PerkinElmer) for 15 min. Cells are washed and chased in assay media with test agent or control agent for 0 to 23 hours in the presence and absence of brefeldin A. At each time point, cells are harvested and lysed in RIPA, and CFTR was immunoprecipitated with M3A7 (Millipore). Samples are separated by SDS/PAGE and analyzed by autoradiography. Radioactivity is quantified by PhosphorImager analysis (GE Healthcare). Quantification of immature CFTR at various time points during the 180-min chase in cells pretreated with vehicle or test agent in the presence and absence of brefeldin A. Data are then fitted with exponential functions (GraphPad) to determine the half-life of corrected CFTR at the cell surface.

If cells treated with a test agent produce more mutant CFTR protein than cells treated with a negative control agent, than this is indicative that the test agent is a candidate corrector agent.

### Example 4

### Ubiquitination Assays

In order to assess the effects of a test agent on ubiquitination of mutant CFTR, an assay is performed in which changes in the ubiquitination of mutant CFTR are assessed in the presence or absence of a test agent.

Corrector Effects on CFTR Ubiquitination.

HEK293 expressing ΔF508-CFTR are treated overnight with DMSO, 3 μM lumacaftor, or 5 μM test agent in the presence or absence of 3 μM lumacaftor. Twenty-four hours later whole cell samples are harvested and polyubiquitinated proteins are selectively isolated using TUBE (Tandem Ubiquitin Binding Entity) affinity resin (Lifesensors Inc.). Western blot analysis is carried out with anti-CFTR or anti-polyUb antibodies.

If cells treated with the test agent have altered ubiquitination patterns or amounts of mutant CFTR as compared to cells treated with a negative control agent, then this is indicative that the test agent is a candidate corrector agent.

### Example 5

### Chloride Transport

In order to assess the effects of a test agent on CFTR chloride transport, Ussing chamber recording analysis is performed.

Primary HBE cell cultures during test agent incubation are maintained in DMEM/F12, Ultroser G (2.0%; catalog no. 15950-017; Pall), fetal clone II (2%), insulin (2.5 μg/mL), bovine brain extract (0.25%; kit CC-4133, component CC-4092C; Lonza), hydrocortisone (20 nM), triiodothyronine (500 nM), transferrin (2.5 μg/mL: catalog no. 0030124SA; Invitrogen), ethanolamine (250 nM), epinephrine (1.5 μM), phosphoethanolamine (250 nM), and retinoic acid (10 nM). The primary HBE cell cultures are grown on Snapwell cell culture inserts (Costar) and maintained at 37° C. before recording in the presence or absence of test agent, a positive control (e.g. lumacaftor) or DMSO. The cell culture inserts are mounted into an Ussing chamber (VCC MC8; Physiologic Instruments) to record the transepithelial current IT in the voltage-clamp mode (0 mV). For FRT cells, the basolateral membrane is permeabilized with 270 μg/mL nystatin, and a basolateral-to-apical chloride gradient is established. The basolateral bath solution contains (in mM) 135 NaCl, 1.2 CaCl₂, 1.2 MgCl₂, 2.4 K₂HPO₄, 0.6 KHPO₄, 10 Hepes, and 10 dextrose (titrated to pH 7.4 with NaOH). The apical NaCl is replaced by equimolar sodium gluconate (titrated to pH 7.4 with NaOH). For HBE cells, the IT is measured in the presence of a basolateral to apical chloride gradient. The basolateral solution contains (in mM) 145 NaCl, 3.3 K₂HPO₄, 0.8 KH₂PO₄, 1.2 MgCl₂, 1.2 CaCl₂, 10 glucose, 10 Hepes (adjusted to pH 7.35 with NaOH) and the apical solution contained (in mM) 145 sodium gluconate, 3.3 K₂HPO4, 0.8 KH₂PO4, 1.2 MgCl₂, 1.2 CaCl₂, 10 glucose, 10 Hepes (adjusted to pH 7.35 with NaOH). All recordings are digitally acquired using Acquire and Analyze software (version 2; Physiologic Instruments). Cell surface turnover of ΔF508-CFTR is determined by first incubating ΔF508-HBE for 48 h with 3 μM lumacaftor and then measuring the forskolin-stimulated I_{T} at the indicated times 0 to 48 h after lumacaftor washout (data from single donor lung; n=6). Activity at various time points are then fitted with exponential function (GraphPad) to determine the half-life of corrected CFTR at the cell surface.

If a test agent induces an increase in forskolin-stimulated I_{T} in the cell cultures, then this is indicative that the test agent is a candidate corrector agent.

### Example 6

### Channel Gating

In order to assess the effects of a test agent on the channel gating of a mutant CFTR at the cell surface, single-channel patch clamp recording analysis is utilized.

The single-channel activity of ΔF508-CFTR and CFTR in cells treated with or without a test agent, lumacaftor or DMSO is measured by using excised inside-out membrane patch recordings as previously described using an Axopatch 200B patch-clamp amplifier (Axon Instruments) (1). The pipette contains (in mM) 150 N-methyl-D-glutamine, 150 aspartic acid, 5 CaCl₂, 2 MgCl₂, and 10 Hepes (adjusted to pH 7.35 with Tris base). The bath contains (in mM) 150 N-methyl-D-glucamine-C1, 2 MgCl₂, 5 EGTA, 10 NaF, 10 TES, and 14 Tris base (adjusted to pH 7.35 with HCl). After excision, CFTR is activated by adding 1 mM Mg-ATP and 75 nM PKA (Promega). The pipette potential is maintained at 80 mV. The Pₒ for CFTR and test agent-corrected and uncorrected ΔF508-CFTR is estimated based on the number of channels in the patch following ivacaftor (1 μM) addition.

If a test agent induces an increase in channel gating activity of the CFTR mutants in a cell, then this is indicative that the test agent is a candidate corrector agent.

### Example 7

### Proteolysis Analysis

In order to assess the effects of a test agent on the proteolytic degradation resistance of a mutant CFTR, a proteolytic degradation analysis assay is utilized.

Twenty-four hours before treatment, HEK-293 cells expressing ΔF508-CFTR or CFTR are plated to 60% confluence in six T225 flasks. The next day, three flasks are treated with test agent, a positive control (e.g. lumacaftor) or with DMSO. Cells are incubated for 24 h in 5% CO2 at 37° C. Each flask is washed once with 10 mL PBS solution and then incubated in 10 mL of Versene (cat. no. 15040; Gibco) for 5 min at room temperature. The cells are dissociated by tapping the flask. Three flasks are combined and the cells were pelleted at 1,500 rpm for 5 min in 4° C. The cell pellet is suspended in 20 mL of sucrose buffer (250 mM sucrose, 10 mM Hepes, pH 7.2) with protease inhibitor mixture. The cells are lysed by nitrogen cavitation at 300 psi for 5 min. Cell lysates are spun down at 2,900 rpm to remove the nuclei. The supernatant is then spun at 34,000 rpm in an ultracentrifuge for 1 h. The pellet is washed in sucrose buffer to remove protease inhibitors and resuspended in 100 μL. Protein concentration is determined using the BCA method. All microsomes are stored at −70° C. Stock of proteomics-grade trypsin (cat. no. T6567; Sigma) is made up in trypsin buffer (40 mM Tris, pH 7.4, 2 mM MgCl₂, 0.1 mM EDTA) and diluted to the following concentrations: 960, 480, 240, 120, 60, 30, and 15 μg/mL. Thirty-five micrograms of protein is resuspended in trypsin buffer to a final volume of 10 μL for each trypsin concentration. Ten microliters of trypsin is added to each tube and incubated for 15 min on ice. The reaction is stopped with 5 μL of 5 mM EDTA and 1 mM PMSF. Ten microliters of 2× Tris-glycine SDS buffer containing 10% β-mercaptoethanol is added to the samples and incubated for 5 min at 37° C. Samples are run on 4% to 20% Tris-glycine gel and transferred onto nitrocellulose. The membrane is blocked for 1 h in 5% milk with PBS plus 0.1% Tween. Membrane is treated in primary antibody overnight at 4° C. NBD-1 is probed by using the CFTR antibody 660 and NBD-2 was probed by using the CFTR antibody 596 (provided by John R. Riordan, University of North Carolina, Chapel Hill, N.C.). Blots are developed by enhanced chemiluminescence and quantified by using NIH ImageJ analysis of scanned films.

If a test agent increases CFTR resistance to proteolysis, then this is indicative that the test agent is a candidate corrector agent.

### Example 8

### Ivacaftor Sensitivity

In order to determine whether a CFTR mutant is sensitive to ivacaftor potentiation, an ivacaftor sensitivity assay is utilized. Human CF subjects having ivacaftor sensitive CFTR mutants would be amenable to a combination therapy of ivacaftor and a corrector agent.

FRT or HBE cells expressing a CFTR mutant are grown on Transwell cell culture inserts (Costar) and maintained at 37° C. before recording. Various concentrations of test agents are then added to the basolateral medium for a period of 18-24 hours prior to recording. The cell culture inserts are mounted into an Ussing chamber (MUsE; Vertex Pharmaceuticals Inc.) to record the transepithelial current in the voltage-clamp mode (0 mV). For FRT cells, the basolateral membrane is permeabilized with 270 μg/mL nystatin, and a basolateral-to-apical chloride gradient was established. The basolateral bath solution contains (in mM) 135 NaCl, 1.2 CaCl2, 1.2 MgCl2, 2.4 K2HPO4, 0.6 KHPO4, 10 Hepes, and 10 dextrose (titrated to pH 7.4 with NaOH). The apical NaCl is replaced by equimolar sodium gluconate (titrated to pH 7.4 with NaOH). For HBE cells, the I_{T} is measured in the presence of a basolateral to apical chloride gradient. The basolateral solution contains (in mM) 145 NaCl, 3.3 K2HPO4, 0.8 KH2PO4, 1.2 MgCl2, 1.2 CaCl2, 10 glucose, 10 Hepes (adjusted to pH 7.35 with NaOH) and the apical solution contains (in mM) 145 sodium gluconate, 3.3 K2HPO4, 0.8 KH2PO4, 1.2 MgCl2, 1.2 CaCl2, 10 glucose, 10 Hepes (adjusted to pH 7.35 with NaOH). CFTR chloride channel currents are elicited by addition of 10 μM forskolin and allowed to reach steady-state. To determine whether the current elicited by forskolin could be further potentiated by ivacaftor, 3 μM ivacaftor in the presence of 10 μM forskolin is added. All recordings are digitally acquired using Acquire and Analyze software (version 2; Physiologic Instruments).

If the forskolin-elicited current is potentiated by ivacaftor, the CFTR mutant protein is ivacaftor-sensitive.

### Examples 9-20

### Materials and Methods

Plasmids, Antibodies, and Reagents

CFTR expression plasmids pcDNA3.1(+)-CFTR and pcDNA3.1(+)ΔF508-CFTR have been described elsewhere (Meacham et al., 2001, Nat Cell Biol, 3:100-5; Younger, et al., 2006, Cell, 126:571-82). CFTR constructs representing CF-disease causing point mutants or truncated biogenic intermediates are made using the QuikChange protocol (Stratagene). The CFTR antibody used in this study is MM13-4 (N-terminal tail epitope) from Upstate Biotechnology. Use of lumacaftor in experiments with cultured cells is previously described (Van Goor, et al., 2011, PNAS, 108: 18843-38).

Cell Culture and Transfection

HEK293 cells from ATCC are maintained in Dulbecco's Modified Eagle's medium (DMEM; GIBCO) supplemented with 1% fetal bovine serum (Hyclone) and antibiotics (100 units/ml penicillin and 100 μg/ml streptomycin; GIBCO) at 37° C. in an atmosphere of 5% CO₂. Cell transfections are performed using Effectene reagent (Qiagen). The empty pcDNA3.1(+) vector is used to ensure equal microgram quantities of DNA are used in all transfection reactions.

Analysis of CFTR Biogenesis-CFTR Steady State Levels

Steady-state levels of CFTR and its mutants are determined by western blot analysis. HEK293 cells are transiently transfected with the indicated plasmids (Grove, et al., 2011, Mol Biol Cell, 22: 301-14). The transfected cells are allowed to recover for approximately 18 hrs before addition of DMEM and then supplemented with lumacaftor or DMSO (control). The cells are incubated with the correctors for 24 hrs before isolating the cells for western blot analysis. The harvested cells are diluted with 2×SDS sample buffer (100 mM Tris-HCl (pH 6.8)/4% SDS/0.05% Bromophenol Blue/20% glycerol), sonicated, and heated at 37° C. prior to resolving the proteins on SDS-PAGE gels. The proteins are transferred to nitrocellulose membranes and the membranes are probed with the designated antibodies. Tubulin is used to indicate loading controls.

Analysis of CFTR Biogenesis-CFTR Processing Efficiency

CFTR processing efficiency is measured by pulse chase analysis (Grove, et al., 2011, Mol Biol Cell, 22: 301-14). Transiently transfected HEK293 cells are allowed to recover for 18 hrs. The cells are then incubated with DMEM supplemented with lumacaftor or DMSO for 2 hrs. Next, cells are starved in methionine-free MEM (Sigma) for 30 min, pulse labeled for 30 min with ³⁵S-methionine (100 μCi/6 well; 1200 Ci/mmol; ICN Radiochemicals) and then chased for the indicated amount of time. Lumacaftor is also included in the media during these steps of the pulse chase reaction. Cells are then lysed in PBS buffer supplemented with 1% Triton (PBS-T (1%)), 1 mM PMSF, and Complete protease inhibitor cocktail (Roche). Soluble lysates are obtained by centrifugation at 20,000 rpm for 10 min in a Beckman Allegra 64R centrifuge. Lysates are normalized to contain the same total amount of protein. ³⁵S-labeled CFTR is immunoprecipitated by incubation with a polyclonal anti-CFTR antibody directed against the N-terminus followed by addition of a 50% Protein G bead slurry. The beads are washed with PBS-T (1%) supplemented with 0.2% SDS, the bound CFTR is eluted with 2× sample buffer, and the samples are heated at 55° C. for 10 min. The samples are analyzed by SDS-PAGE and visualized by autoradiography.

Limited Proteolysis of CFTR

The content of 6 wells of a 6 well plate containing HEK293 cells were transfected with 1 μg of the indicated CFTR plasmid (Rosser et al., 2008). 24 hours post-transfection the cells were harvested in citric saline and lysed in PBS-Tr (0.1%) for 1 hour at 4° C. Lysates were cleared by centrifugation at 20,000 rpm for 10 min in a Beckman Allegra 64R centrifuge. Supernatants were removed and total microgram quantities of protein were determined by the DC Bio Rad protein determination assay. Cell lysates were then diluted to a concentration of 2 μg/ml and trypsin was added at the indicated final concentrations. The cleavage reactions incubated on ice for 15 minutes, and were then quenched by addition of Complete Protease Inhibitor (Roche) and Trypsin Inhibitor. Sample Buffer was added to a final 1× concentration, and samples were run on 12.5% SDS PAGE gels. Gels were transferred to nitrocellulose and probed with N-terminal tail CFTR antibody (MM13-4 1:1000 dilution).

Co-Expression of N- and C-Terminal Domains of CFTR

Cells were transfected with pcDNA3.1-CFTR837X (1 μg) and pcDNA3.1-CFTR837-1480 (1 μg), individually or in combination, and were used to evaluate the impact of lumacaftor on the assembly of CFTR's membrane domains (Rosser et al., 2008). Reactions were balanced with pcDNA3.1 such that all transfections were performed with equal microgram quantities of DNA. 24 hours post-transfection cells were harvested with citric saline, diluted in 2× sample buffer, sonicated for 10 seconds, and warmed to 37° C. for 10 minutes prior to loading on 10% SDSPAGE gels. Proteins were transferred to nitrocellulose using a Bio-Rad mini gel wet transfer apparatus. Blots were blocked in blocking buffer containing 10% fat-free milk and 0.1% Triton-X 100 in PBS and probed with anti-CFTR monoclonal N-terminal tail (MM13-4 1:1000 dilution).

Electrophysiology

Ussing chamber techniques with Fisher Rat Thyroid cells that are stably transfected with the indicated form of CFTR are used to record the transepithelial current (IT) resulting from CFTR-mediated chloride transport (Van Goor, et al., 2009, PNAS, 106: 18825-30). Standard conditions for Ussing chamber electrophysiological with different forms of CFTR measured forskolin (10 μM)-stimulated chloride transport that peaked at 205.5 μA/cm2 for normal CFTR.

### Example 9

### Lumacaftor Stabilizes Folding of MSD1 in CFTR Protein

To localize the region in CFTR on which lumacaftor acts to correct ΔF508-CFTR misfolding, immunoblot and pulse-chase studies are used to monitor the impact of the drug on the accumulation of a set of CFTR and ΔF508-CFTR fragments that expose surfaces present on CFTR folding intermediates.

A set of CFTR fragments with domain boundaries that permitted them to accumulate in unstable or stable states are expressed in HEK293 cells, and the effect of 5 μM lumacaftor on the accumulation of different fragments is determined. As a point of reference, the impact of the proteasome inhibitor bortezomib on CFTR fragment accumulation is also measured.

Cells are treated for 4 hours with bortezomib or 18 hours at 37° C. with lumacaftor. The amount of CFTR fragment accumulation is determined by immunoblot.

CFTR³⁷⁰ and CFTR⁵³⁰ fragments accumulate to relatively low amounts and bortezomib increases their accumulation by 10-fold. In contrast, accumulation of CFTR⁴³⁰ and CFTR⁶⁵³ is 10-fold higher than that of CFTR³⁷⁰, and is relatively insensitive to proteasome inhibition. Thus, the region defined as MSD1 by sequence analysis, is unstable when expressed in cultured cells. Information in the region that lies between residues 371-430 is required for TM1-TM6, which is located between residues 83 to 358, to assume a relatively stable conformation.

CFTR⁶⁵³ contains MSD1 and full length NBD1, and may be relatively stable because it possesses the information required for NBD1 to fold and make proper contacts with MSD1. In contrast, CFTR⁵³⁰ is truncated in the middle of NBD1, and thus resembles a misfolded protein that would be expected to be an ERAD substrate.

CFTR³⁷⁰ has a short half-life and its accumulation is increased dramatically by inhibition of the proteasome. Lumacaftor has no effect on CFTR³⁷⁰ steady amounts or half-life. In contrast, lumacaftor has a positive impact on the steady-state accumulation of CFTR⁴³⁰, CFTR⁵³⁰ and CFTR⁶⁵³. In addition, pulse-chase studies show that the increase in the steady-state amounts of CFTR⁴³⁰ and CFTR⁶⁵³ by lumacaftor correlate with an increase in their half-life.

Lumacaftor has similar effects on the accumulation of normal and ΔF508 mutant forms of CFTR⁵³⁰ and CFTR⁶⁵³, and so lumacaftor does not act in a manner that is specific for the presence of ΔF508. In addition, lumacaftor has no impact on the half-life of CFTR³⁷⁰, and so lumacaftor does not cause CFTR⁴³⁰ accumulation via general inhibition of protein quality control. CFTR⁴³⁰ contains MSD1, plus a segment of NBD1 that lies between residues 391 and 430.

Lumacaftor at 5 μM maximally stimulates CFTR escape of CFTR from the ER in HEK293 cells as indicated by accumulation of the C-form. At this concentration, lumacaftor has no effect on the expression of folding and degradation factors that influence that fate of nascent CFTR.

### Example 10

### Lumacaftor Acts Through MSD1 of CFTR

To determine if lumacaftor acts on MSD1 or the MSD1:NBD1 interface the minimal region of CFTR whose conformation is impacted by lumacaftor is analyzed. This process is aided by the analysis of sequence alignments between human CFTR and the bacterial ABC transporters, Sav1866 and MsbA, whose 3D structures are known (Mornon et al., (2009) Cellular and molecular life sciences: CMLS 66, 3469-3486). The homology model of CFTR based in the inward or closed conformation also provides structural information on the TM spans of MSD1 as well as the N-terminal tail and the structure of the regions between residue 370 and the start of NBD1 at position 391 (Mornon et al., 2009). This information is absent from the CFTR homology model based on the Sav1866 structure in the outward or open conformation (Serohijos et al., (2008) Proc Natl Acad Sci USA 105, 3256-3261; Mornon et al., (2008) Cell Mol Life Sci 65, 2594-2612). Thus, the sequence alignments and the homology model for CFTR based on the MsbA inward structure is used as a guide to study basic features of MSD1 folding. This model predicts that the N-terminal tail of CFTR is located between residues 1-82 and TM1-6 of MSD1 is located between residues 83-358.

To evaluate whether the 362-380 helix is critical for MSD1 folding, the accumulation and sensitivity to lumacaftor of CFTR³⁷³, CFTR³⁷⁵, and CFTR³⁸⁰ is analyzed. CFTR³⁷³ accumulation is similar to that of CFTR³⁷⁰ and insensitive to lumacaftor. CFTR³⁷⁵ accumulation is similar to CFTR³⁷⁰, but its amounts are increased around 6-fold by lumacaftor. Accumulation of CFTR³⁸⁰ is 4-fold higher than that of CFTR³⁷⁰ and its accumulation is also increased 6-fold by lumacaftor. Maximal accumulation of CFTR³⁸⁰ occurs at 5 μM lumacaftor.

In addition, pulse-chase studies show that the half-life of CFTR³⁸⁰ is similar to that of CFTR⁴³⁰ and CFTR⁶⁵³ and that it is increased several fold by lumacaftor.

To further examine the role of lumacaftor on residues 371-375, the effects of lumacaftor on residues 371-375 in full-length CFTR folding is explored. An in frame deletion of residues 371-375 is constructed and the accumulation and sensitivity to lumacaftor of Δ371-375 CFTR is determined. Δ371-375 CFTR does not accumulate in the C-form and the presence of lumacaftor does not promote escape of the B-form from the ER.

To further test the role of residues in the 362-380 helix in its interaction with lumacaftor, the amino acid F374 is mutated to an alanine and its effect on CFTR biogenesis is examined. F374 CFTR accumulates in the B-form, but its folded C-form is not detected. Lumacaftor does not stimulate conversion of the B-form of F374A CFTR to the C-form. Mutation or deletion of residues in 362-380 helix cause biogenic defects in CFTR that are not repaired by lumacaftor. In addition, lumacaftor does not stabilize purified NBD1 or ΔF508-NBD1.

### Example 11

### MSD1 is Stabilized in a Protease Resistant Conformation by Lumacaftor

To analyze how the 362-380 helix impacts the conformation of MSD1 to confer its sensitivity to lumacaftor, the conformation of CFTR³⁷⁰ and CFTR³⁸⁰ in the presence and absence of lumacaftor by limited proteolysis with trypsin is probed. In the presence and absence of lumacaftor, CFTR³⁷⁰ is completely digested by low amounts of trypsin. CFTR³⁸⁰ conformation is partially resistant to trypsin digestion. CFTR³⁸⁰ is cleaved by trypsin, but a protease resistant species with an apparent molecular weight of around 22 Kd is detected. Lumacaftor protects around 60% of the CFTR³⁸⁰ from cleavage of the 22 Kd species.

The impact of lumacaftor on the conformation of MSD1 from ΔF508-CFTR is also analyzed. The pattern of low molecular weight trypsin resistant fragments liberated from CFTR 380 is compared to those liberated by ΔF508-CFTR. A 22kd fragment is also liberated from full-length ΔF508-CFTR. Lumacaftor increases the quantity of the protease resistant 22 kd fragments that is liberated from ΔF508-CFTR by trypsin.

CFTR³⁸⁰ contains 48 different trypsin cleavage sites and CFTR³⁷⁰ contains 46. The monoclonal antibody MM13-4 utilized to detect trypsin digested CFTR recognizes the peptide RKGYRQRLELSD located at position 25-36 in the N-terminus. CFTR³⁸⁰ contains trypsin cleavage sites throughout its sequence, but has a cluster of 6 sites between residues 242 and 258. This cluster is located in the coupling helix that extends into the cytosol for TM4. Cleavage of CFTR here generates a CFTR fragment that is detected by the N-terminal tail antibody that has an apparent molecular weight of around 22-23 Kd.

### Example 12

### Lumacaftor Corrects Functional Defects Caused by Missense Mutations in MSD1

A collection of CFTR mutants (E56K and P67L, E92K, L206W and V232D) is containing disease related mutations in N-terminal regions of CFTR that encompass cytosolic and membrane spanning regions of MSD1 are generated. These CFTR mutants are expressed in polarized FTR cells, which is required for electrophysiological analysis of repaired Cl− channel function. The ability of lumacaftor to restore Cl⁻ channel function of different CFTR mutants is determined. Lumacaftor restores Cl⁻ channel function to near or greater than wild type for 4 of the 5 MSD1 mutants tested. E56K and P67L are located in the N-terminal tail of CFTR and are positioned in the model of the CFTR structure near the 362-380 helix. E92K is located in TM1 and L206W is located in TM3. V232D is located in TM4 in a region of MSD1 that is not in the vicinity of the other mutations or the 362-380 helix, and correction of its functional defects by lumacaftor is relatively modest.

As a point of comparison, the impact of lumacaftor on functional defects caused by disease related missense mutations in NBD1 and the ICL4/NBD1 interface is also determined. The functional correction is modest when compared to that with the CFTR MSD1 mutants.

### Example 13

### The Nature of Disease Related Mutations in CFTR Limit Lumacaftor Efficacy

To test whether the differences in efficacy and potency of lumacaftor in functional correction of E92K-CFTR and ΔF508-CFTR is due to these mutations generating different rate limiting steps in CFTR biogenesis, the efficacy of lumacaftor on folding of the double mutant E92K-ΔF508-CFTR is determined. The dose response of E92K-ΔF508-CFTR resembles that of E92K-CFTR, with maximal folding correction occurring at 30 μM of lumacaftor, but the efficacy of folding correction is similar to that for ΔF508-CFTR.

### Example 14

### Stabilization of the NBD1: ICL4 Interface Increases Lumacaftor Efficacy on ΔF508-CFTR

To determine whether defective interactions between ΔF508-NBD1 and ICL4 limit the efficacy of lumacaftor on ΔF508-CFTR, the impact of the intragenic suppressor mutation V510D, which restores contacts between NBD1 and ICL4 (36), on the efficacy of lumacaftor action on ΔF508-CFTR is evaluated. In addition, R1070 in ICL4 is predicted to make backbone contacts with F508 (Thibideau et al., (2010) J Biol Chem 285, 35825-35835). Mutation of R1070W is thought to overcome this folding defect and provide an alternative mode for binding of NBD1 to ICL4 to enhance ΔF508-CFTR folding (Thibideau et al., 2010). Thus, whether the introduction of R1070W or V510D into ΔF508-CFTR increased the efficacy of lumacaftor action is addressed. R1070W and V510D alone corrects ΔF508-CFTR folding to around the same degree as lumacaftor. Lumacaftor has an additive effect with R1070W, as it is able to restore ΔF508-R1070W-CFTR folding to around 35% of control. In pulse-chase studies the addition of lumacaftor stimulates folding of the nascent 35S-ΔF508-V510D-CFTR and 35S-ΔF508-R1070W-CFTR.

The V510D mutation has previously been proposed to generate a salt bridge with R1070 to partially restore contacts between ΔF508-NBD1 and ICL4. Thus, the influence of a R1070A mutation on the action of lumacaftor on ΔF508-V510D-CFTR is examined. R1070A reduces by around 75% the ability of lumacaftor to correct ΔF508-V510D-CFTR folding. V510D is also proposed to improve the folding efficiency of ΔF508-NBD1.

### Example 15

### Lumacaftor Stabilizes N-Terminal CFTR Fragments that Contain MSD1

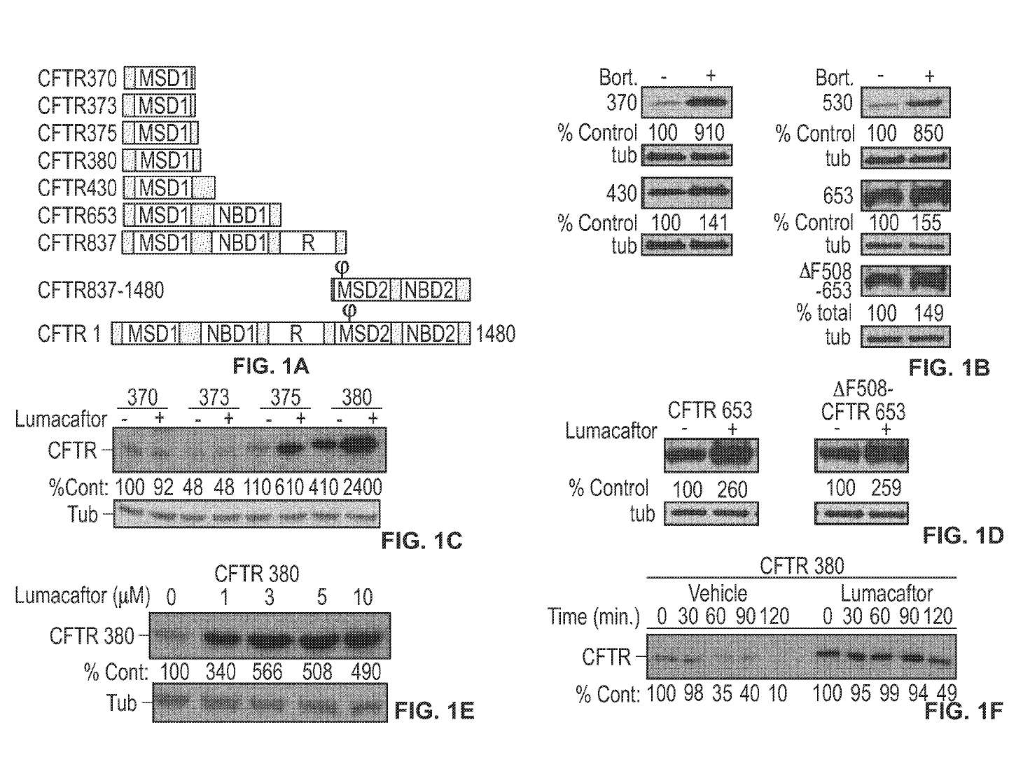

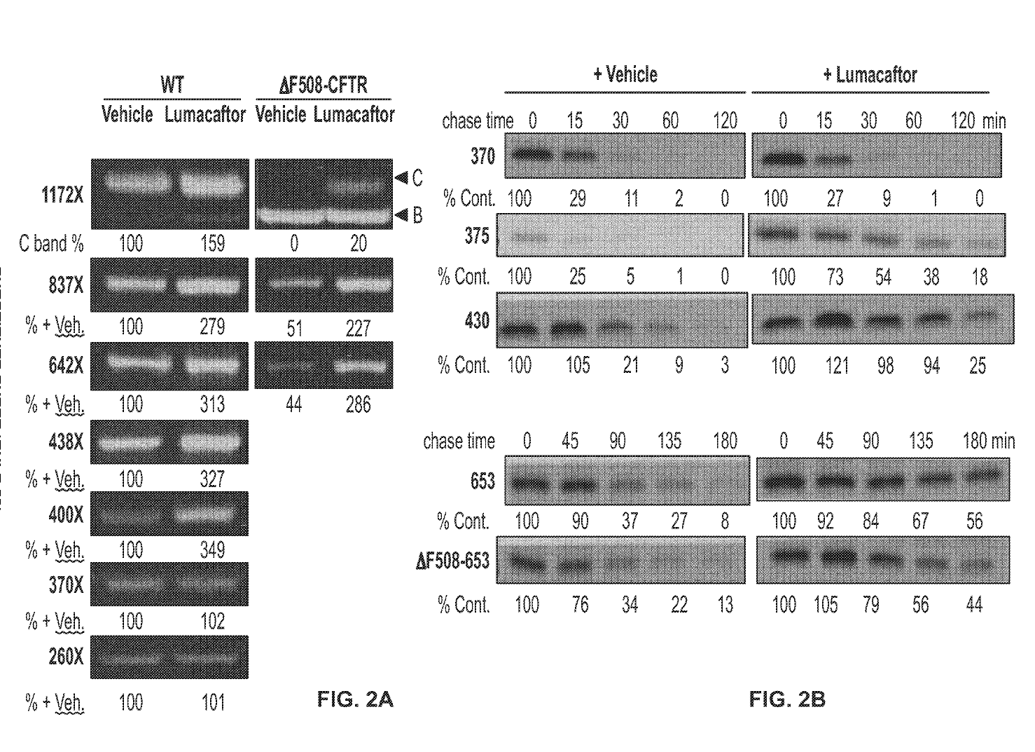

To identify regions of CFTR required for lumacaftor activity, different length CFTR fragments were expressed in HEK-293 cells and their steady-state accumulation and half-life were quantified in biochemical assays (FIGS. 1A and B; FIG. 2). Since CFTR folding initiates cotranslationally and channel assembly is completed post-translationally (Higgins, 1992, Annual Review of Cell Biology, 8:67-113), CFTR fragments which may resemble folding intermediates have been used to study CFTR biogenesis. The underlying rationale is that differences in accumulation or half-life between different length CFTR fragments are believed to reflect differences in the stability of the protein conformation and resistance to endoplasmic reticulum associated degradation (ERAD). Consistent with this, CFTR fragments CFTR³⁷⁰ and CFTR⁵³⁰, which accumulated at markedly lower levels compared to CFTR⁴³⁰ and CFTR⁶⁵³, were more susceptible to proteasome inhibition by bortezomib (FIG. 1B). Differences in accumulation and halflife of CFTR fragments have been used successfully to identify Hsp70- and calnexin-dependent steps in CFTR folding and degradation pathways (Farinha and Amaral, 2005, Mol Cell Biol, 25:5242-52; Lukacs and Verkman, 2012, EMBO Journal, 13:6076-86; Meacham et al., 1999, EMBO Journal, 18:1492-1505; Okiyoneda et al., 2004, Mol Biol Cell, 15:563-574), to identify defective interactions between MSD1, NBD1 and MSD2 as an underlying cause of premature degradation of ΔF508-CFTR (Cui et al., 2007, J Mol Biol, 365:981-994; Du and Lukacs, 2009, Molecular Biology of the Cell, 20:1903-1915; Younger et al., 2006, Cell, 126:571-582), and to identify MSD2 as the site of action of the CFTR corrector, Corr-4a. In addition, co-expression of non-overlapping N- and C-terminal CFTR fragments in cells led to an increase in chloride transport, suggesting that these individual CFTR fragments were able to co-assemble and form a functional CFTR channel (Csanady et al., 2000, J Gen Physiol, 116:477-500).

The shortest length CFTR fragment affected by lumacaftor was CFTR³⁷⁵, which contains only MSD1 (FIG. 1C). Levels of longer length CFTR fragments were also increased by lumacaftor, including CFTR fragments that contained NBD1 with the ΔF508 mutation (FIG. 1D; FIG. 2). Lumacaftor did not increase the stability of CFTR 837-1480, which contains only MSD2 and NBD2 (FIG. 2). In dose response studies using CFTR³⁸⁰, the maximal effective concentration of lumacaftor was 3 μM, which is similar to that observed for full-length ΔF508-CFTR (FIG. 1E) (Van Goor et al., 2011, PNAS, 108:18843-48). In pulse chase studies, the half-lives of CFTR³⁷⁵, CFTR³⁸⁰, CFTR⁴³⁰, and CFTR⁶⁵³ were increased in the presence of lumacaftor as compared to untreated controls (FIG. 1F, and FIG. 2), indicating that the increase in steady-state accumulation caused by lumacaftor is associated with an increase in the stability of the individual CFTR fragments. The inability of lumacaftor to increase the accumulation of CFTR³⁷⁰ suggests that lumacaftor does not act as a proteasome inhibitor, as bortezomib was able to increase the stability of this CFTR fragment (FIG. 1B). Taken together, these data indicate that the shortest-length CFTR requirement for lumacaftor action contains only MSD1. Moreover, these data suggest that lumacaftor increases the stability of the protein conformation of MSD1, and as a consequence, its resistance to ERAD.

### Example 16

### Lumacaftor Alters the Protein Conformation of MSD1

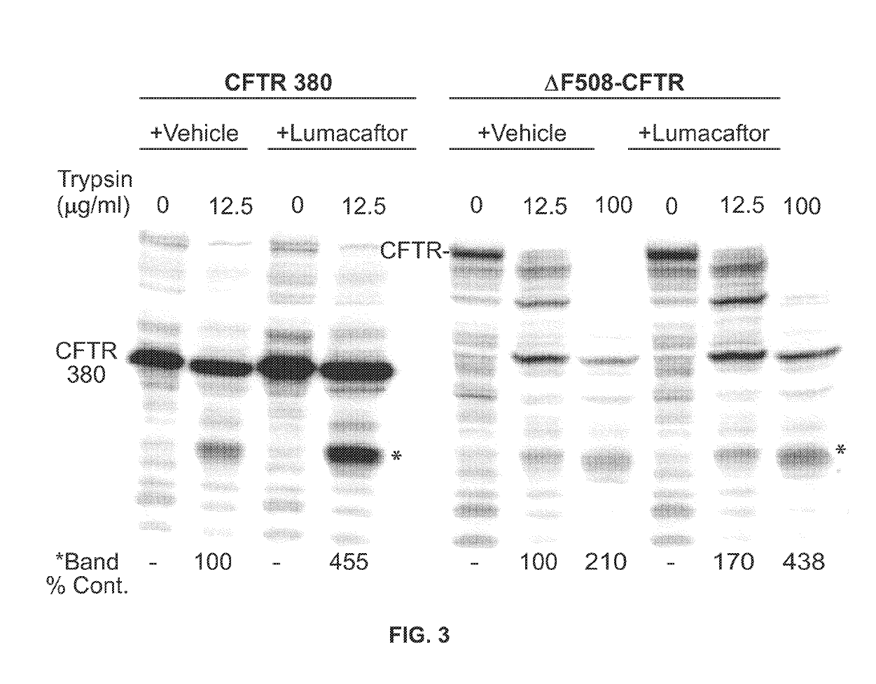

To test if lumacaftor alters the protein conformation of MSD1 to result in a more stable folded form that is resistant to ERAD, the compound's ability to protect a subdomain of MSD1 from proteolytic digestion was tested using limited proteolysis (FIG. 3). This technique is based on the premise that folded proteins are more compact and therefore typically more resistant to proteolytic digestion than unfolded or partially folded proteins and has been used to probe differences in protein folding between wild-type and ΔF508-CFTR as well as between uncorrected and lumacaftor corrected ΔF508-CFTR. For these studies, CFTR³⁸⁰ was used as it was one of the most stable CFTR fragment following treatment with lumacaftor. In cells expressing CFTR³⁸⁰ or full-length ΔF508-CFTR, treatment with lumacaftor increased the liberation of a protease-resistant species with an apparent molecular weight of 22 Kd that was detected with an antibody directed against the N-terminal tail of CFTR. Taken together, data presented in FIGS. 1 and 3 suggest that lumacaftor alters the protein conformation of MSD1 to suppress folding defects in ΔF508-CFTR.

### Example 17

### Residues 371-380 Help MSD1 Fold to a Conformation that May be Acted Upon by Lumacaftor

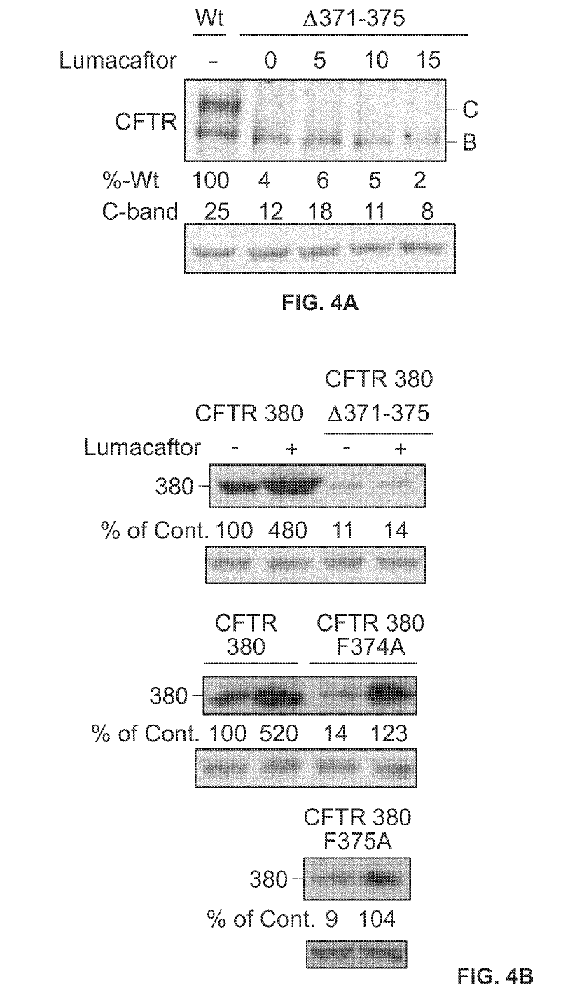

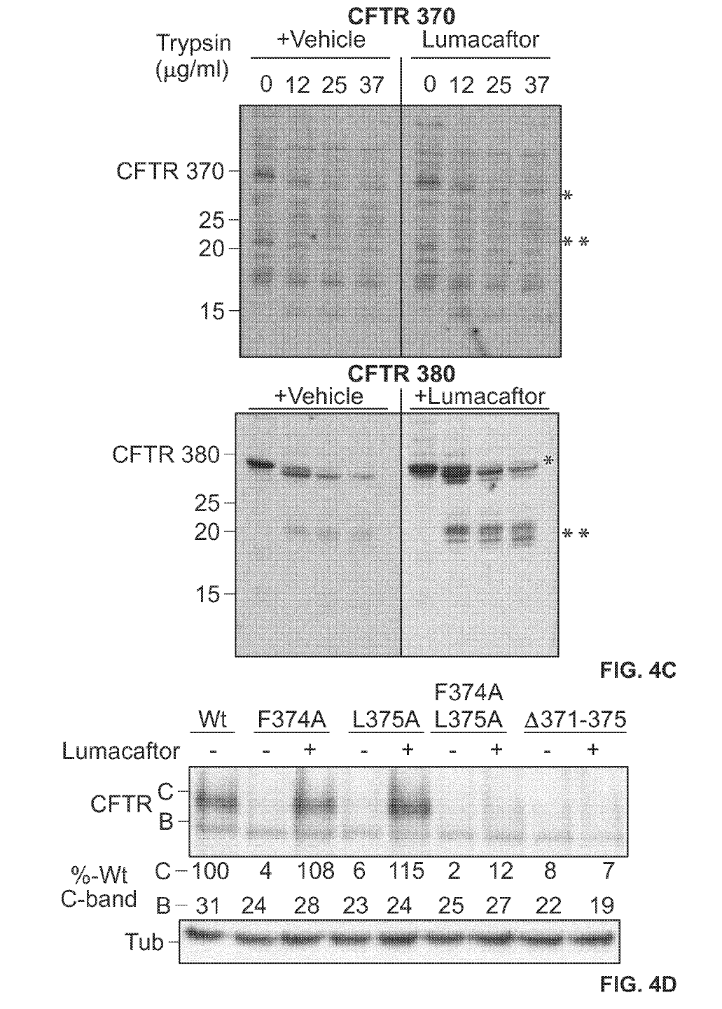

The data above indicated that residues 374-375 are required for sensitivity of MSD1 fragments to lumacaftor, whereas residues 376-380 appear to aid in folding of MSD1 to a more stable form (FIG. 1C). Deletion of residues 371-375 from full length CFTR caused a severe folding defect, resulting in little to no mature form (C-band) and eliminating sensitivity to lumacaftor (FIG. 4A). Similarly, Δ371-375 CFTR³⁸⁰ accumulated at 10% of CFTR³⁸⁰ levels and was insensitive to lumacaftor (FIG. 4B). Thus, residues 371-375 are important for proper folding of CFTR and their deletion caused a biogenic defect that was not corrected by lumacaftor.

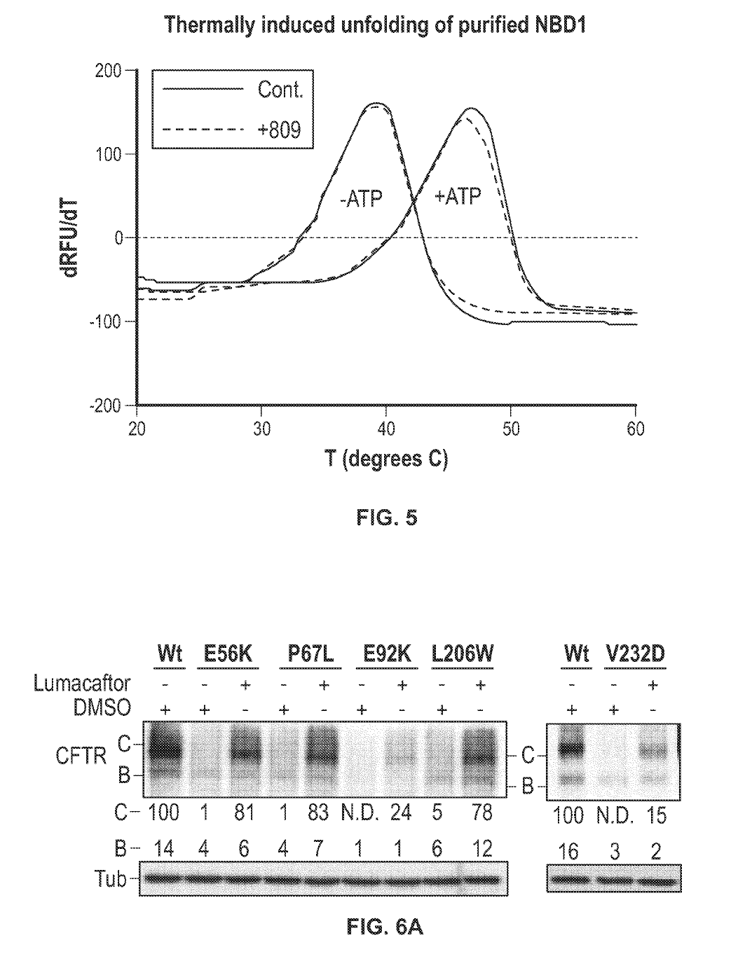

To determine if residues 370-380 are critical for folding of MSD1 to a conformation that is sensitive to lumacaftor, the resistance of CFTR³⁷⁰ and CFTR³⁸⁰ to digestion by trypsin was compared (FIG. 4C). CFTR³⁷⁰ was completely digested by trypsin and was not protected by lumacaftor, whereas CFTR³⁸⁰ adopts a conformation that is partially resistant to trypsin digestion and was protected from digestion by lumacaftor. Thus, residues 371-380 are important for folding of MSD1 to a biochemically stable conformation. Once translation of CFTR past residue 375 occurs, all the forms of CFTR examined are acted upon by lumacaftor (FIG. 1-4). In addition, lumacaftor had no detectable impact on the stability of NBD1, as assessed by its lack of impact on the thermally induced unfolding of purified NBD1 (FIG. 5).

Given that the extension of CFTR³⁷³ by two residues confers sensitivity of MSD1 fragments to lumacaftor, residues F374 and L375 might be involved in binding of lumacaftor. Despite the severe biogenic defects exhibited by F374A-CFTR, L375A-CFTR and the double mutation F374A/L375A-CFTR (FIG. 4A) and F374A CFTR³⁸⁰ and L375A CFTR³⁸⁰ (FIG. 4B), lumacaftor almost completely suppressed the biogenic defects caused by the F374A and L375A mutations at the 5 μM concentration that maximally suppressed folding defects in ΔF508-CFTR (FIGS. 4A and B). Thus, while residues 371-375 are important for CFTR folding, mutation of either F374 and L375 does not alter the potency or efficacy of lumacaftor, suggesting that neither of these specific residues was critical for binding of lumacaftor to CFTR.

### Example 18

### Lumacaftor Suppresses Folding Defects in CFTR Caused by Disease Related Mutations in MSD1

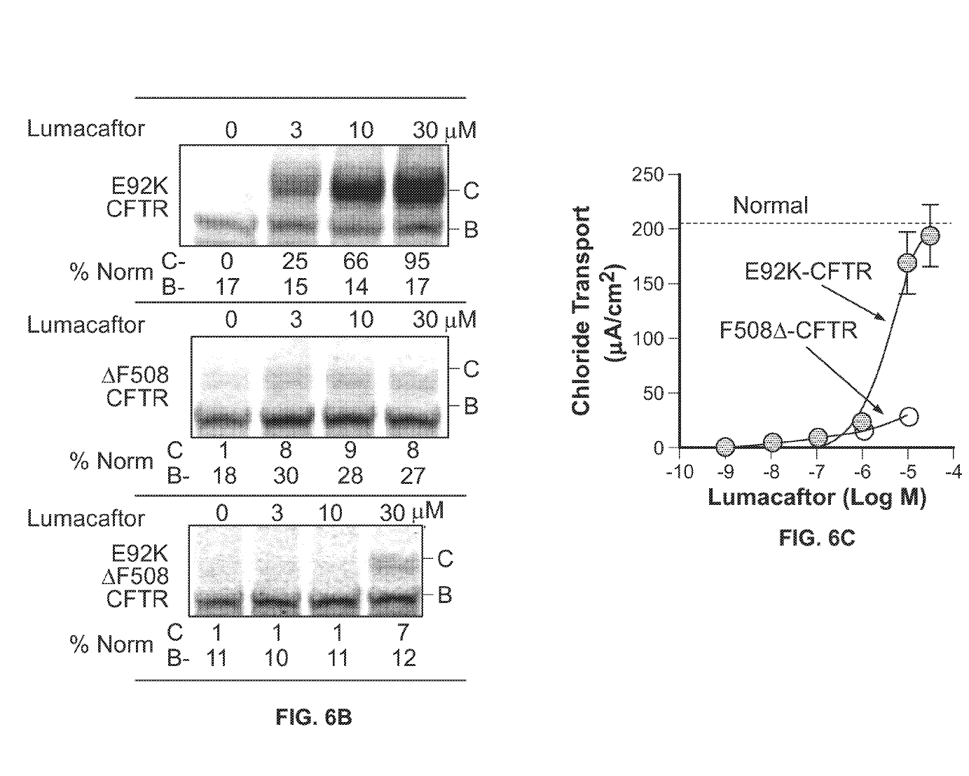

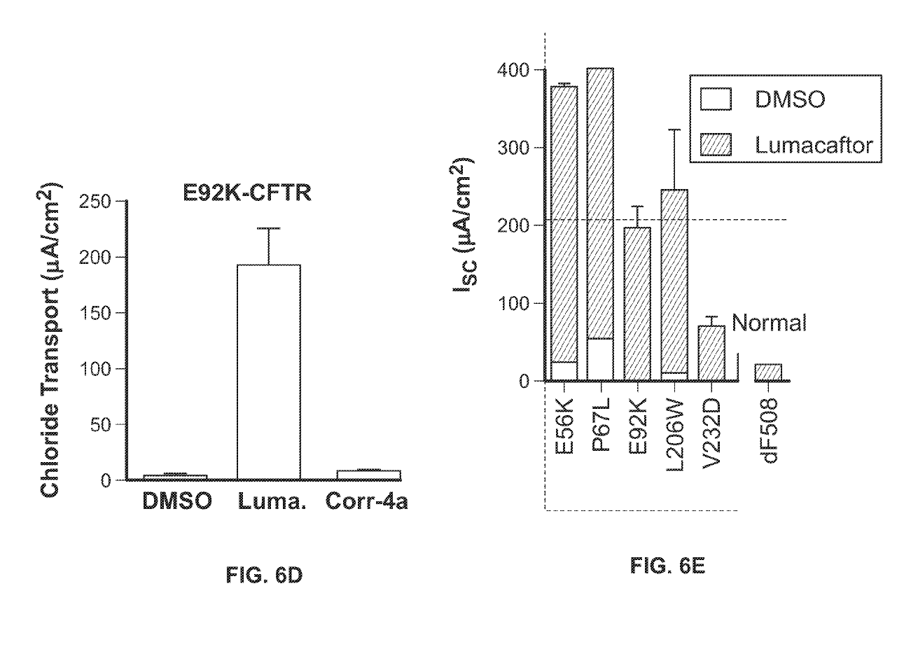

There are several CF-associated mutations in MSD1 that cause defects in CFTR processing and function: N-terminal tail (E56K and P67L), TM1 (E92K), TM2 (L206W) and TM4 (V232D) (FIG. 6A-E). The severe folding (FIG. 6A-B) and functional (FIG. 6E) defects exhibited E56L, P67L and L206W were completely corrected by 5 μM lumacaftor. In contrast, 5 μM lumacaftor only partially restored folding and function to E92K and V232D (FIGS. 6A and E). Lumacaftor demonstrated reduced potency for E92K-CFTR relative to ΔF508-CFTR, both for correcting folding and function (FIGS. 6B and C), yet was able to fully restore E92K-CFTR at 30 μM. However, the corrector Corr-4a could not restore E92K-CFTR function (FIG. 6D).

V232D-CFTR was the least responsive to lumacaftor, and higher concentrations of the compound did not restore function beyond the 25% of normal CFTR observed in the presence of 5 μM lumacaftor. Taken together with the observation that Corr4a restored V232D-CFTR biogenesis and function to normal levels (Caldwell et al., 2011, American Journal of Physiology. Lung cellular and molecular physiology, 301:L346-352), these data suggest that correctors such as lumacaftor and Corr4a can act to selectively suppress folding defects in CFTR caused by different disease related mutations in MSD1.

Since E92K-CFTR was corrected to normal levels of function, but ΔF508 was corrected to about 15% of normal function, the impact of lumacaftor on the double mutation, E92K/ΔF508-CFTR was tested (FIG. 6B). The effect of lumacaftor on accumulation levels of the C-form of E92K/ΔF508-CFTR was consistent with the dose response for E92K-CFTR while the level of efficacy was consistent with that for ΔF508-CFTR. Thus, the E92K mutation causes a folding defect in E92K/ΔF508-CFTR that requires a higher compound concentration, but the folding defects caused by ΔF508 limit the efficacy of lumacaftor. These data support the concept that lumacaftor action on MSD1 aids in suppression of some but not all of the folding defects caused by ΔF508.

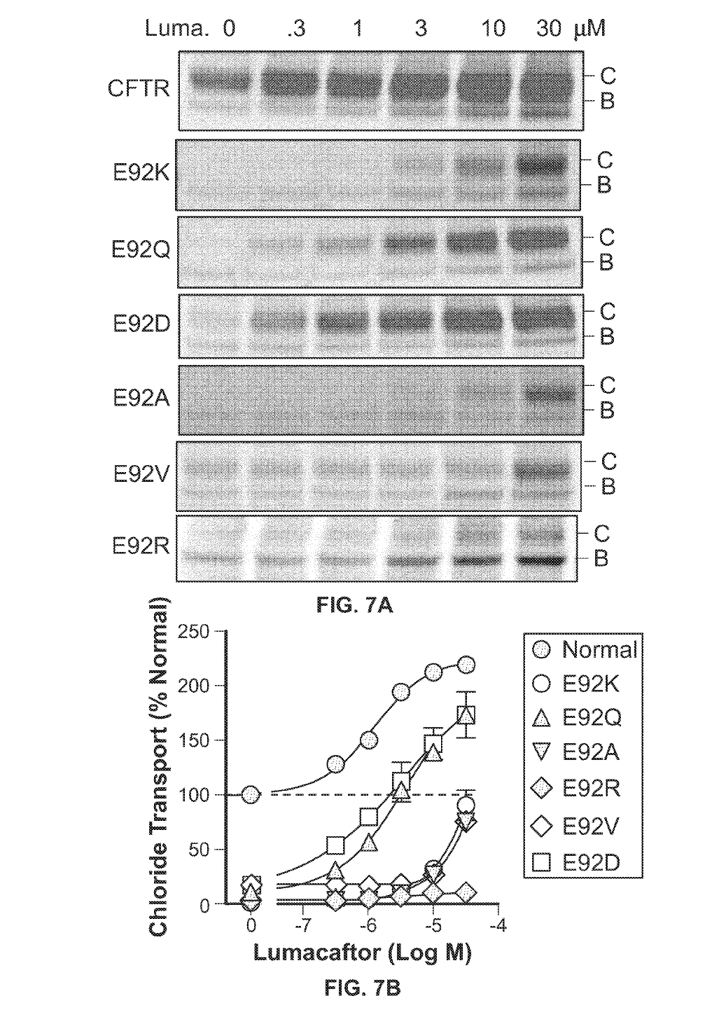

Lumacaftor is highly efficacious at correction of folding defects in CFTR caused by some, but not all of the missense mutations in MSD1 that were evaluated. E92K is unique among these mutants as it alters the potency of lumacaftor, yet the biogenic defects caused by this mutation are completely corrected by lumacaftor. Mutational analysis of E92 suggest that mutation of this residue disrupts a salt bridge in MSD1 that is required for CFTR folding (FIG. 7). E92 may, therefore, not be directly involved in binding lumacaftor, and instead, appears to be required for folding of MSD1 to a conformation that binds lumacaftor with high affinity.

### Example 19

### Interdomain Communication is Required for Lumacaftor to Enhance CFTR Folding

Lumacaftor was efficacious at correcting the folding defects caused by missense mutations in MSD1, and its ability to stabilize MSD1 appears to partially suppress the folding defects caused by ΔF508 from NBD1. Lumacaftor efficacy was limited by folding defects that appear to occur downstream of its effects on MSD1. Since lumacaftor restored the function of some MSD1 CFTR mutants to normal levels in model cells, there is potential that it could act in concert with an additional corrector to restore ΔF508-CFTR function to normal levels (Mendoza et al., 2012, Cell, 148:164-174; Rabeh et al., 2012, Cell, 148:150-163; Van Goor et al., 2011). Understanding the nature of the defective folding step(s) that limit lumacaftor efficacy on ΔF508-CFTR may aid in the development of such corrector combinations. The biogenic defects in ΔF508-CFTR that might limit lumacaftor efficacy include: 1) defective assembly caused by increased thermodynamic instability of ΔF508-NBD1 relative to NBD1 (Wang et al., 2010, Protein Science: a publication of the Protein Society, 19:1932-47), or 2) defective assembly of ΔF508-NBD1 into a complex with ICL4 of MSD2 caused by loss of the F508 side chain (Serohijos et al., 2008, PNAS, 105:3256-61).

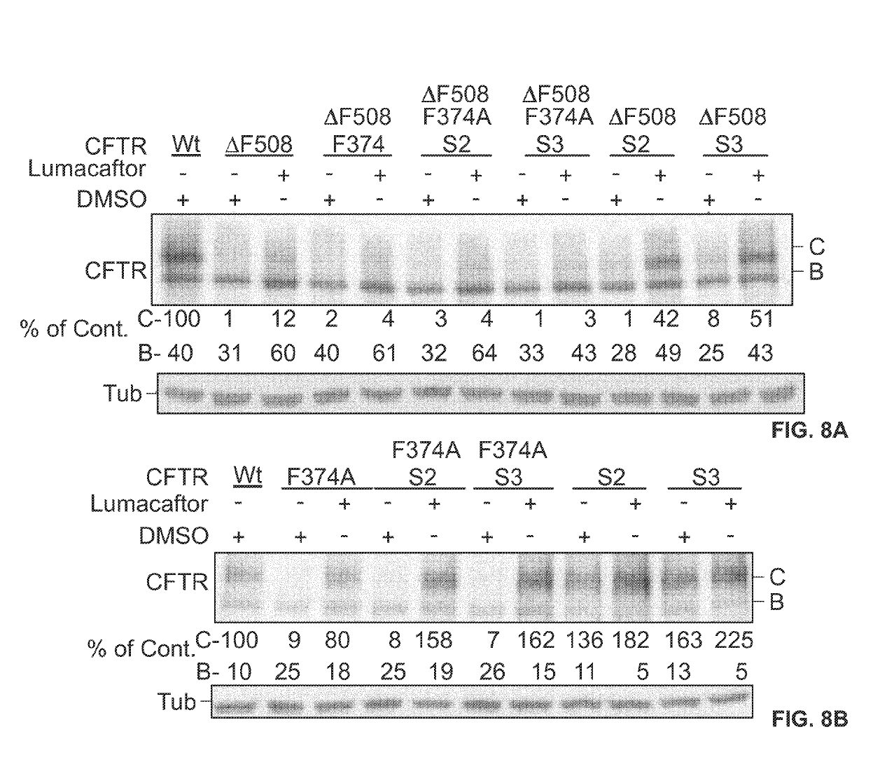

To determine the extent to which thermodynamic instability of ΔF508-NBD1 impacts efficacy of lumacaftor on ΔF508-CFTR, the effect of lumacaftor on a set of intragenic suppressor mutations that increase the solubility of purified NBD1, thereby partially suppressing biogenic defects in ΔF508-CFTR (Amaral and Farinha, 2013, Curr Pharm Des, 19(19):3497-508; Pissarra et al., 2008, Chem Biol, 15:62-69; Teem et al., 1993, Cell, 73:335-46), was examined. These mutations are termed solubilizing (S) mutations and were introduced into NBD1 in different combinations: S2 (F429S, Q637R) and S3 (F429S, F494N, and Q637R). Lumacaftor increased accumulation levels of the C-form of ΔF508-CFTR to around 14% of normal CFTR and the S mutations by themselves have little impact on accumulation of the C-form of ΔF508-CFTR (FIG. 8A). In the presence of lumacaftor, the C-form of S2/ΔF508-CFTR and S3/ΔF508-CFTR accumulated at up to 45% of normal/wildtype levels (FIG. 8A). These data suggest that thermodynamic instability of ΔF508-NBD1 limits the efficacy of lumacaftor on ΔF508-CFTR.

The positive effect of S2 and S3 on ΔF508-CFTR biogenesis was abolished by the F374A mutation, as lumacaftor could not drive high-level accumulation of the C-form of F374A/S2/ΔF508-CFTR or F374A/S3/ΔF508-CFTR. In addition, the F374A mutation hindered lumacaftor from suppressing folding defects in ΔF508-CFTR. In experiments with CFTR, the S2 and S3 mutations by themselves increased C-band accumulation almost 2-fold, and this effect was blocked by F374A (FIG. 8B). In contrast to results with ΔF508-CFTR, lumacaftor restored accumulation of the C-form of F374A/S2-CFTR and F374A/S3-CFTR to levels of S2 CFTR and S3 CFTR under control conditions. F374 is located in the cytosolic linker domain, positioned between TM6 and NBD1, that is required for folding of MSD1 to a conformation that can be modulated by lumacaftor (FIGS. 3 and 4). These data suggest that F374 facilitates inter-domain communication between MSD1 and NBD1 and, further, that allosteric communication of structural information between MSD1 and NBD1 appears critical for lumacaftor correction of ΔF508-CFTR.

### Example 20

### Stabilization of the NBD1:ICL4 Interface Increases Lumacaftor Efficacy on ΔF508-CFTR

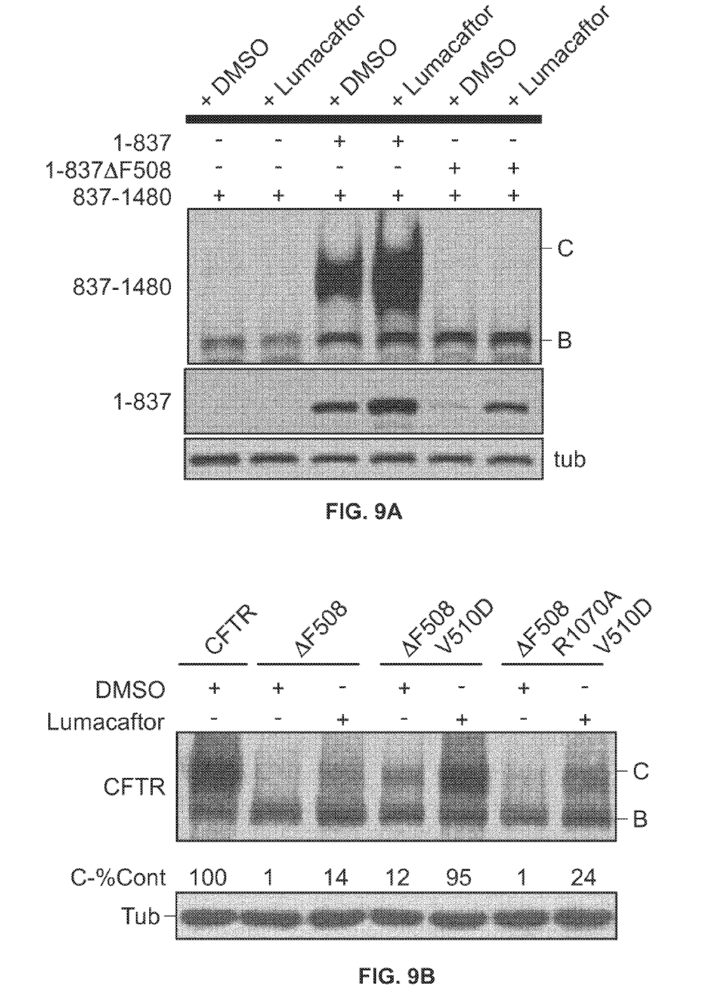

Defective assembly of NBD1 into a complex with solvent-exposed residues on ICL4 hinders ΔF508-CFTR folding (Serohijos, et al., 2008, Proc Natl Acad Sci USA 105:3256-3261). The extent to which this folding defect limits lumacaftor efficacy (FIG. 9) was tested by examining the ability of lumacaftor to enhance the assembly of nonoverlapping N- and C-terminal CFTR fragments into a complex that can escape the ER (FIG. 9A). Fragments of CFTR that contain the N-(1-837) and C-(837-1480) terminus assemble into a complex that folds, escapes the ER, and accumulates as a maturely glycosylated species (Rosser et al., 2008, Mol Biol Cell, 19:4570-79). Thus, if lumacaftor acts on MSD1 to positively impact TM segment assembly it would be expected to increase CFTR fragment assembly. Consistent with previous results (FIGS. 1-3), lumacaftor does not increase accumulation of CFTR⁸³⁷⁻¹⁴⁸⁰ alone (FIG. 9A, lane 1 and 2), but did increase accumulation of CFTR¹⁻⁸³⁷. In addition, lumacaftor stimulated the CFTR¹⁻⁸³⁷-dependent accumulation of the C-form by greater than 2-fold (FIG. 9A, lane 3 vs 4).

Deletion of F508 prevents accumulation of ΔF508-CFTR¹⁻⁸³⁷ (FIG. 9A lane 3 vs 5) and ΔF508-CFTR¹⁻⁸³⁷ does not productively interact with CFTR⁸³⁷⁻¹⁴⁸⁰ (i.e., the C-form does not accumulate in the presence of ΔF508-CFTR¹⁻⁸³⁷). Lumacaftor increased the accumulation of ΔF508-CFTR¹⁻⁸³⁷ by several fold (FIG. 9A, lane 5 vs 6), but did not promote interactions between ΔF508-CFTR¹⁻⁸³⁷ and CFTR⁸³⁷⁻¹⁴⁸⁰ as indicated by a lack of accumulation of a C-form (FIG. 9A, lane 6).

Defective contacts between ΔF508-NBD1 and ICL4 that limit ΔF508-CFTR assembly have been partially restored by introduction of the V510D mutation into NBD1 by permitting the formation of a salt bridge between D510 and R1070 of ICL4 (Wang et al., 2007, J Biol Chem, 282:33247-251) (FIG. 9B). The V510D mutation partially corrects misfolding of ΔF508-CFTR as shown by the C-form for V510D/ΔF508-CFTR (FIG. 9B, lane 4) to levels similar to that for ΔF508-CFTR in the presence of lumacaftor. Lumacaftor stimulated the accumulation of the C-form of V510D/ΔF508-CFTR to the levels observed for normal CFTR.

To determine if formation of a salt bridge between D510 and R1070 was important for this effect, the R1070A mutation was introduced into V510D/ΔF508-CFTR. In the presence of lumacaftor, the accumulation of the C-form of R1070A/V510D/ΔF508-CFTR was reduced by 75% relative to V510D/F508-CFTR (FIG. 9B, lane 7). Lumacaftor was still able to increase folded R1070A/V510D/F508-CFTR to levels that were significantly higher than those for lumacaftor treated ΔF508-CFTR. Since the V510D mutation can modestly improve the thermodynamic stability of purified NBD1 (Lewis et al., 2010, J Mol Biol, 396:406-430; Wang et al., 2010, PNAS, 19:1932-47), the residual lumacaftor corrector function on R1070A/V501D/ΔF508-CFTR could result from the thermodynamic stabilization of NBD1 that would occur in the absence of salt bridge formation between D510 and R1070.

### Example 21

### Lumacaftor Binding to MSD1

To assess the binding of lumacaftor and its analogs to CFTR fragments, photoaffinity labeling experiments were utilized. In these experiments, a tritiated photoactive analog of lumacaftor (“active photoanalog”) was utilized. The active photoanalog was prepared either with a low specific activity (27.2 Ci/mmol) or with a high specific activity (174 Ci/mmol). In the presence of ultraviolet light, the active photoanalog will covalently bind to its binding site on a polypeptide target.

In addition, a photaffinity inactive analog of lumacaftor (“inactive photoanalog”) was also synthesized as a negative control.

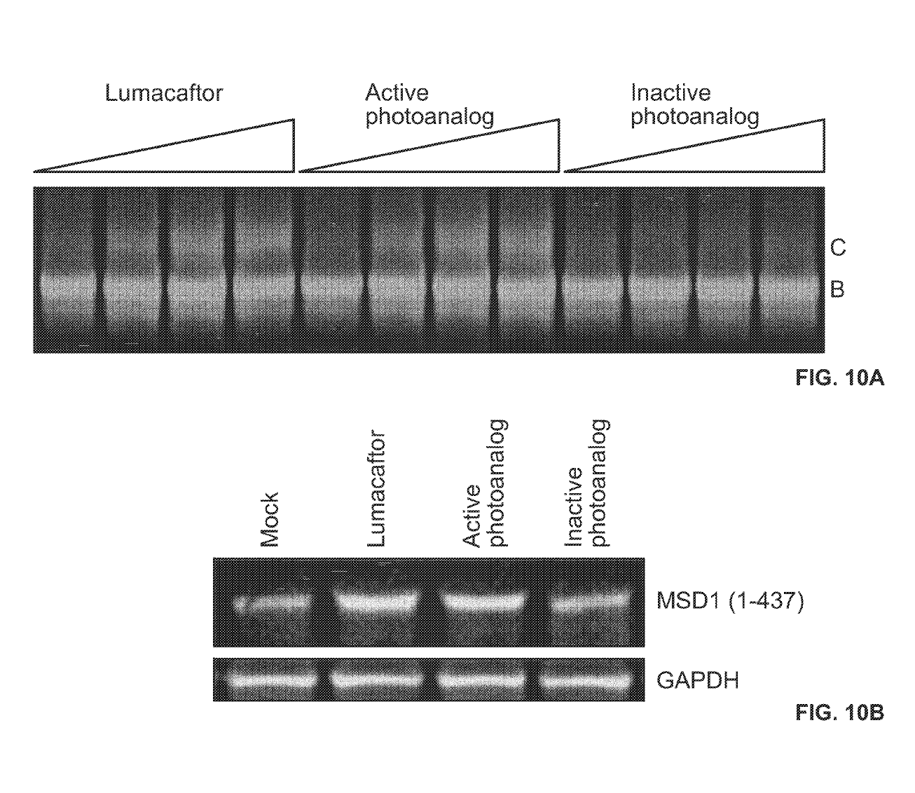

The active photoanalog of lumacaftor corrected the trafficking of ΔF508-CFTR with similar potency and efficacy as lumacaftor (FIG. 10A). In addition, treatment of cells expressing an MSD1 fragment of the CFTR protein (amino acid residues 1-437 of SEQ ID NO: 1) with either lumacaftor or the active photoanalog resulted in an increase in steady state levels of the MSD1 fragment (FIG. 10B). By comparison, the inactive photoanalog of lumacaftor has no apparent effect on ΔF508-CFTR trafficking or on MSD1 fragment steady state levels (FIG. 10).

To determine the specificity of the photoactive analog for binding MSD1 in Sf9 cells, the molecular weight profile of proteins labeled with the photoactive analog was examined. Sf9 cells were cultured in serum free ESF-921 medium (The Expression Systems) supplemented with the surfactant Pluronic F-68 (GIBCO) at 0.1% (vol/vol). Cells were maintained and infected in suspension in a rotary shaker. For infection, cells were seeded at the density of 1.0-1.5×10⁶ cells per ml (>95% viability). Either the sequence for MSD1 (amino acids 1-437 of SEQ ID NO: 1) or MSD2 (amino acids 837-1172 of SEQ ID NO: 1) was amplified by PCR adding 6×His tag at the C-terminus and inserted into pFastBac vector (Invitrogen). Baculovirus were then prepared containing the prepared pFastBac vector. The seeded Sf9 cells were then inoculated with the baculovirus at MOI-5. Forty-eight hours post-infection, the MSD1-transfected or mock-transfected Sf9 cells were seeded at 200 μl of 10⁶ cells/ml into a 96-well plate. The photoactive analog having the low specific activity was then added to each well to a final concentration of 1 μM. The cells were then irradiated for 60 minutes using a handheld ultraviolet lamp self-filtered to 365 nm (Fisherbiotech) suspended 1 cm above the treated cells at room temperature. Irradiated cells were then washed twice with PBS, lysed and processed for SDS-PAGE analysis. Gel areas of specified molecular weight ranges were excised directly from the SDS-PAGE gel, or were transferred onto nitrocellulose membrane first, and membrane slices were counted in a liquid scintillation counter (Beckman, LS 6500).

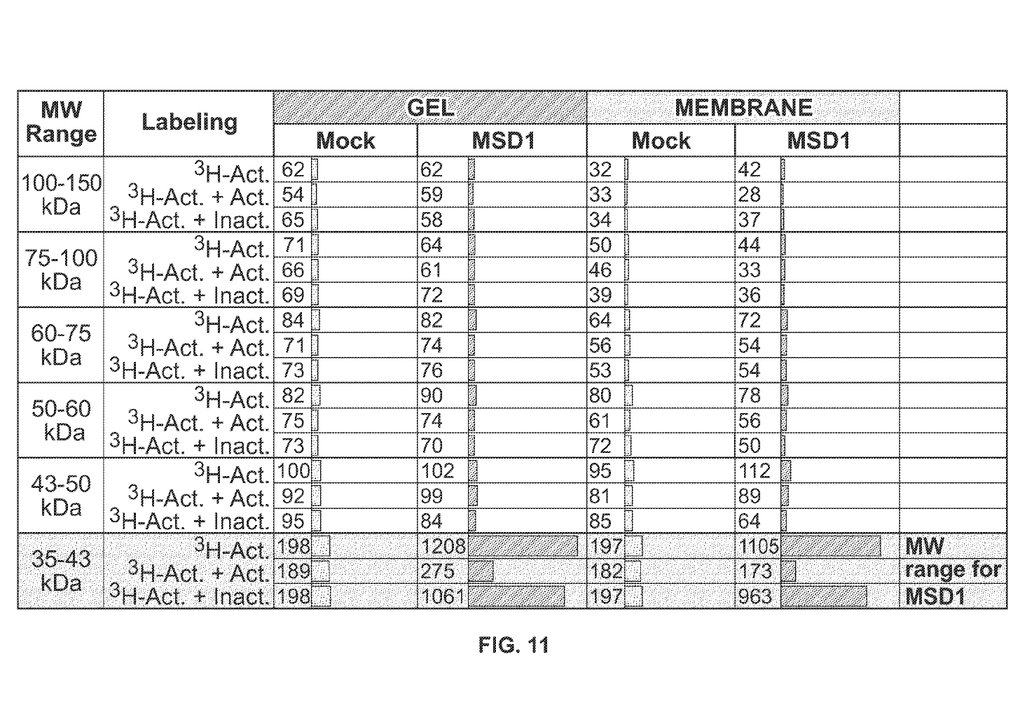

As illustrated in FIG. 11, the tritiated active photoanalog was incorporated predominantly into a peptide having the expected molecular weight of MSD1 protein (i.e., 43 kDa) as compared to control mock-transfected cells. FIG. 11 also illustrates that the addition of 20× excess non-tritiated active photoanalog to the Sf9 cell suspension before UV irradiation greatly reduced the incorporation of tritiated active photoanalog into MSD1 protein. In the absence of UV irradiation, no incorporation of the tritiated active photoanalog was observed (not shown). Moreover, the addition of 20× excess of non-tritiated inactive photoanalog following UV irradiation did not alter the amount of tritiated active photoanalog bound to MSD1 protein (FIG. 11). Tritiated active photoanalog was also bound in a diffuse band with an average molecular weight of 10-15 kDa. However, the binding between the active photoanalog and the 10-15 kDa fragment was deemed to be non-specific, because this binding was similar between mock-transfected and MSD1 expressing cells, and also because this binding was unaffected by the presence of either non-tritiated active photoanalog (20X) or inactive photoanalog (20X) (FIG. 11).

The binding interaction between MSD1 and the active photoanalog was also tested in HEK293 cell lysates. HEK293 cells were cultured in Dulbecco's modified Eagle's medium with 10% fetal bovine serum in a humidified atmosphere of 95% air, 5% CO2 at 37° C. HEK293 cells were stably transfected with a mammalian expression vector encoding an N-terminal fragment of CFTR that contains all of MSD1 tagged with two tandem HA epitopes at the N-terminus and with a hexahistidine tag at the C-terminus (2×HA-438X-6×His) using Polyfect (Qiagen, Inc.). Cells were harvested and lysed in 1% NP-40, 0.5% sodium deoxycholate, 200 mM NaCl, 10 mM Tris, pH 7.8, and 1 mM EDTA plus protease inhibitor cocktail (Roche). Insoluble material was removed by centrifugation (15,000×g, 10 min, 4° C.) before photoaffinity labeling. The active photoanalog (27.2 Ci/mmol) was purchased from Vitrax. Lysates were incubated with 1 μM active photoanalog in a total volume of 100 μl and UV irradiated at 365 nm (Fisher Scientific) for 60 min. From the UV irradiated lysates, the 2×HA-438X-6×His CFTR fragment was immunoprecipitated using the anti-HA antibody. Immunoprecipitates were then separated by SDS-PAGE (4-12% Bis-Tris), transferred onto nitrocellulose, probed with a monoclonal antibody against amino acids 405-438 of CFTR (Univ. North Carolina), then detected using infrared fluorescence (LiCor). Portions of the blot containing the CFTR-MSD1 fragments were excised and measured for radioactivity (LSC, Beckman).

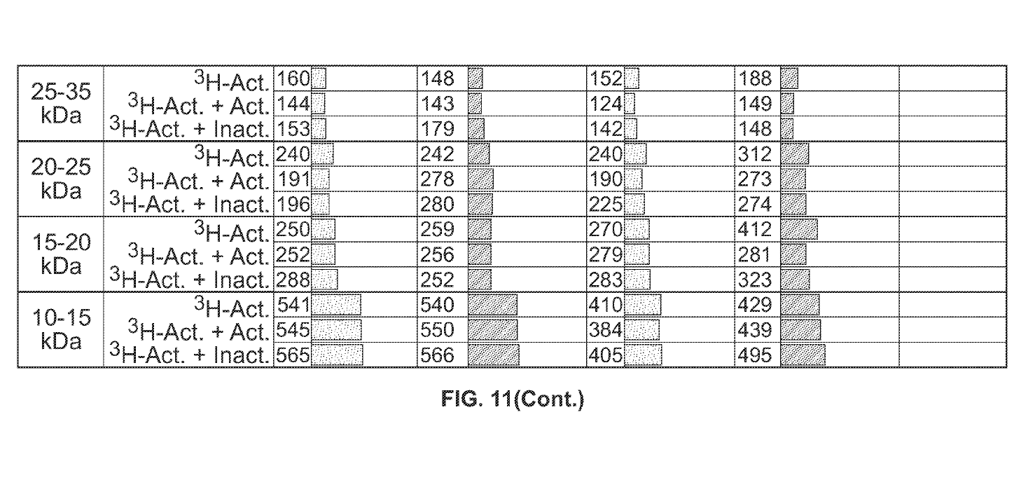

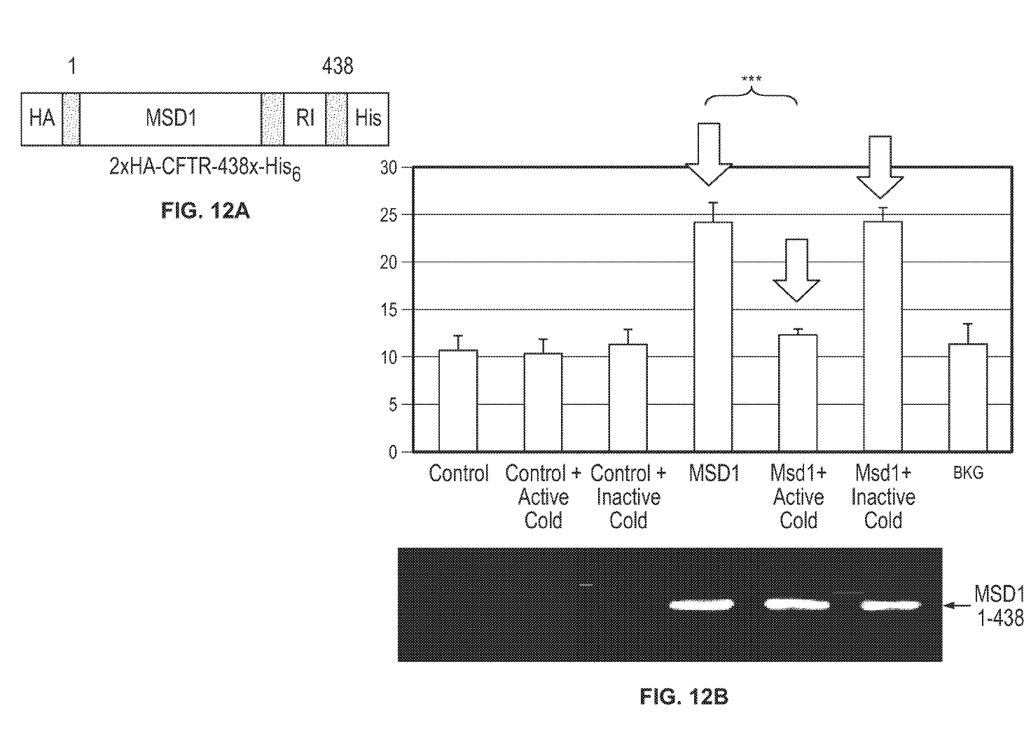

The photoactivatable lumacaftor active analog binds to and crosslinks with MSD1 in HEK293 cell lysates (FIG. 12). This binding is blocked in the presence of 20-fold excess unlabeled active analog, but not in the presence of cold inactive analog (FIG. 12B).

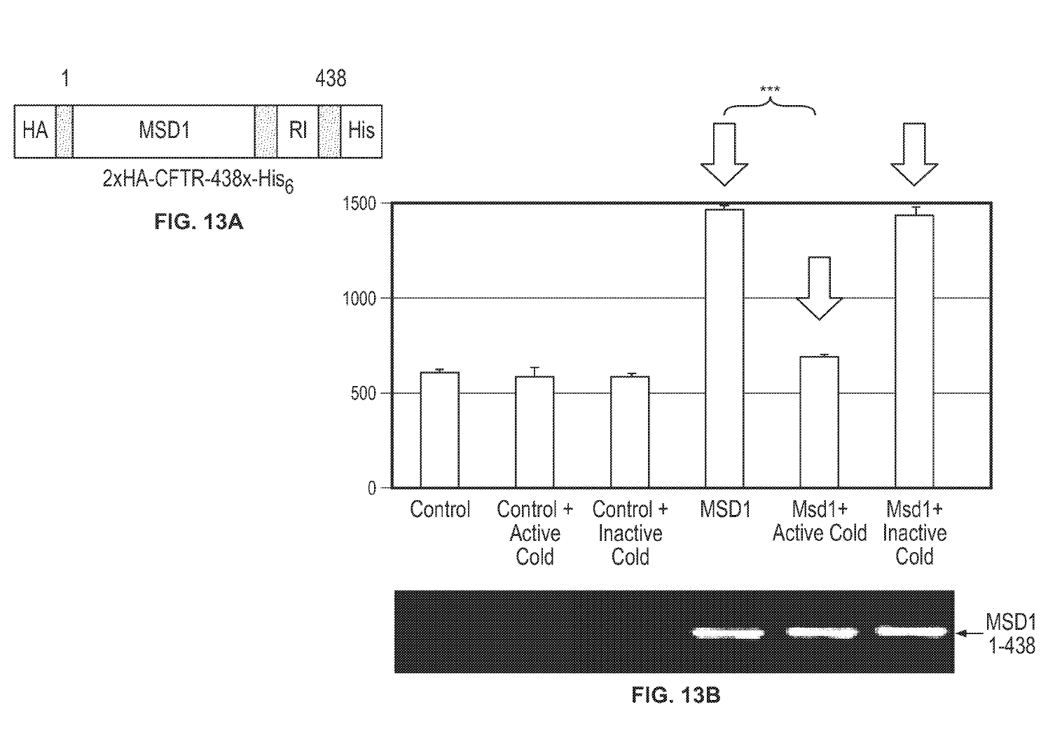

The binding interaction between MSD1 and the active photoanalog was also tested in live Sf9 cells. Sf9 cells were cultured in ESF921 medium (Expression Systems) in a humidified atmosphere of 95% air, 5% CO2 at 27° C. Sf9 cells were infected with a series of baculovirus expression vectors encoding for N-terminal fragments of CFTR that contain all of MSD1 (CFTR³⁷⁶, CFTR³⁹², CFTR³⁸⁵, CFTR⁴³⁸) at an MOI of 5. As a negative control, a fragment containing just MSD2 of CFTR (amino acids 837-1172) was also used. Three days after infection, cells were incubated with 1 μM tritiated photoanalog for 1 hour at 25° C. and then irradiated at 365 nm (Fisher Scientific) for 60 min. Cells were then harvested and lysed in 1% NP-40, 0.5% sodium deoxycholate, 200 mM NaCl, 10 mM Tris, pH 7.8, and 1 mM EDTA plus protease inhibitor cocktail (Roche). Insoluble material was removed by centrifugation (15,000×g, 10 min, 4° C.). Lysates were separated by SDS-PAGE (4-12% Bis-Tris), transferred onto nitrocellulose, probed with a monoclonal antibody against the 6×His Tag, then detected using infrared fluorescence (LiCor). Portions of the blot containing the CFTR-MSD1 fragments were excised and measured for radioactivity (LSC, Beckman). The active photoanalog bound to MSD1 in Sf9 cells (FIG. 13). This binding is blocked in the presence of 20-fold excess unlabeled active analog, but is not blocked in the presence of the cold inactive analog (FIG. 13B).

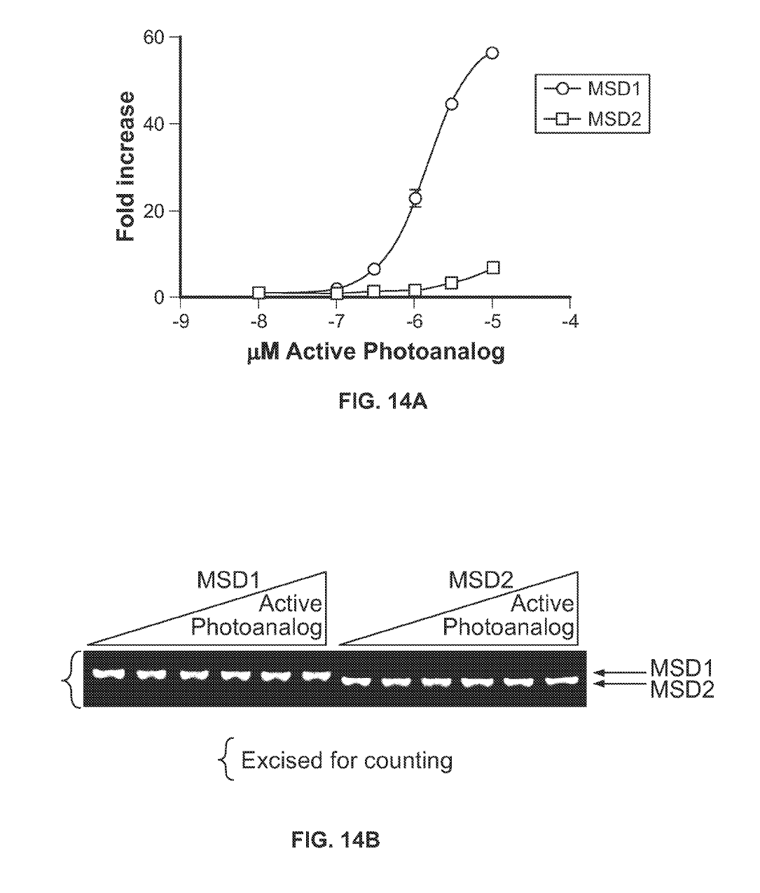

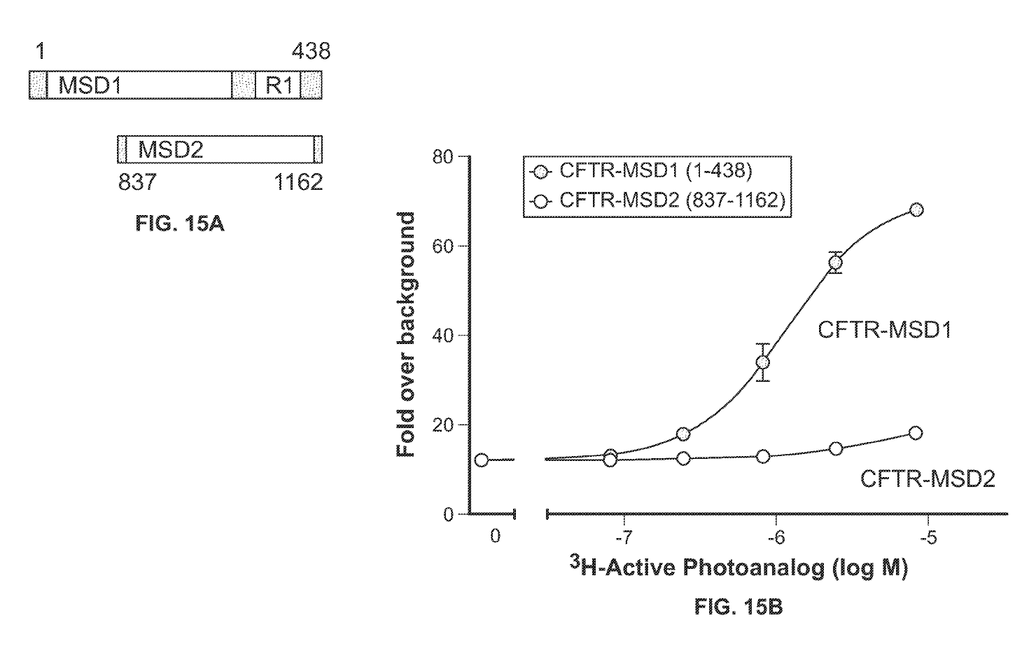

The binding interaction between the active photoanalog and either an MSD1 and an MSD2 fragment was also examined. In one experiment, Sf9 cells expressing either MSD1 or MSD2 fragments of CFTR (as described above) were incubated with different concentrations (0, 0.1, 0.3, 1, 3 or 10 μM) of active photoanalog and then irradiated with ultraviolet light as described above. Photoaffinity labeling of MSD1 increased with increasing concentrations of active photoanalog (FIG. 14). Active photoanalog incorporation into MSD2 was markedly reduced compared to that observed with MSD1 (FIG. 14A). Similar results were observed in a similar experiment in which MSD1 and MSD2 fragments were incubated with different doses of the active photoanalog (FIG. 15).

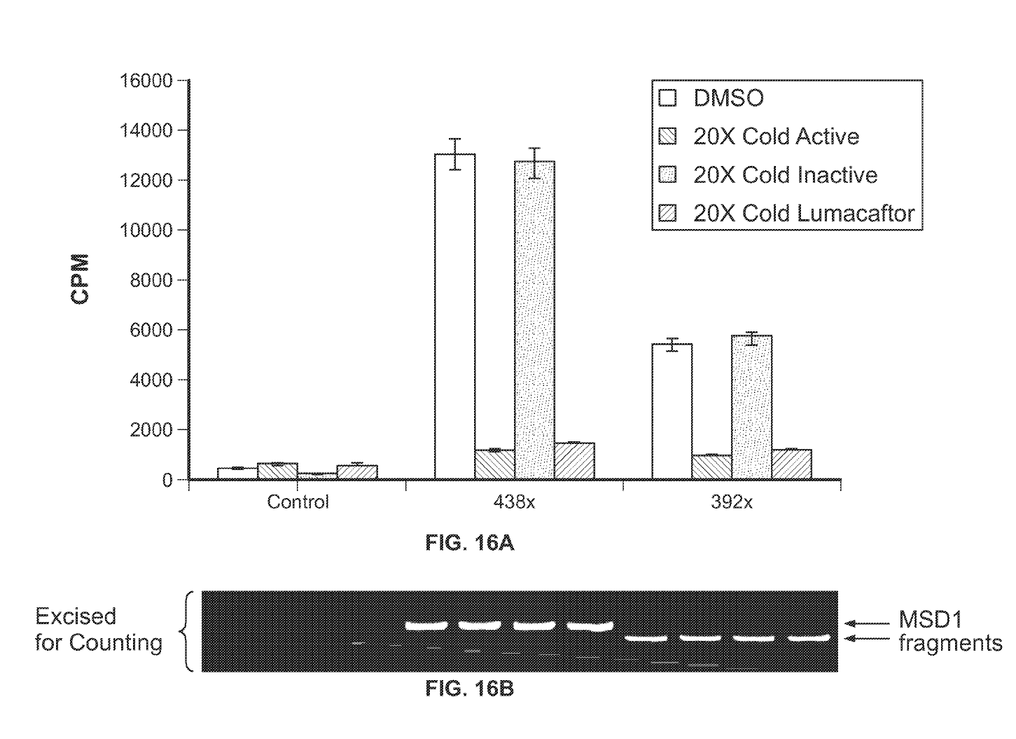

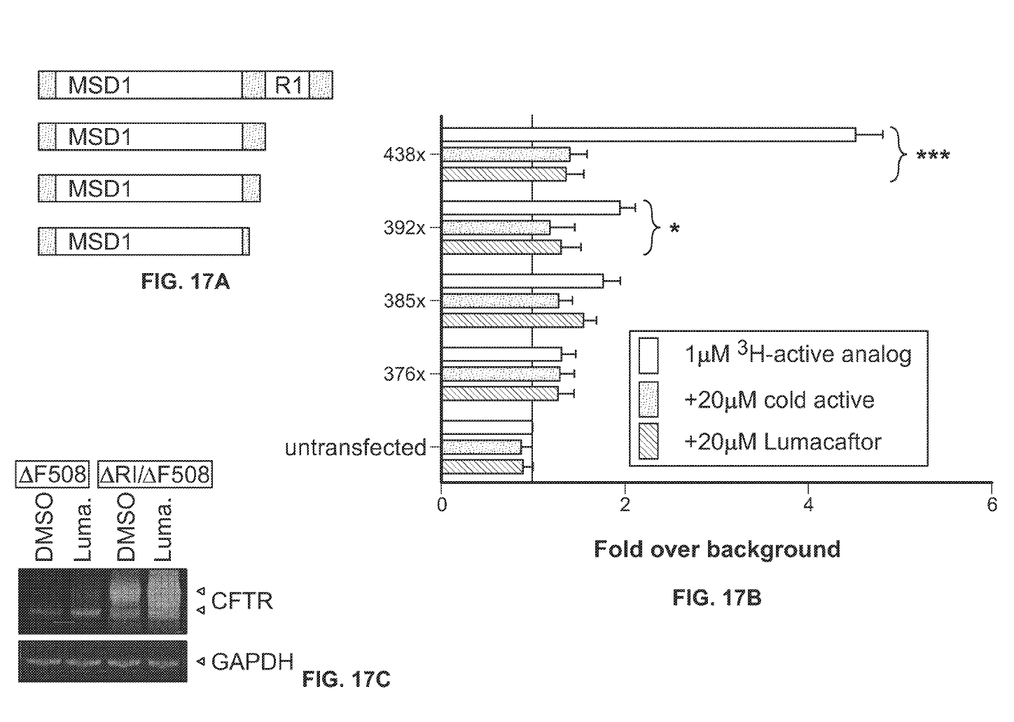

The binding interaction between MSD1 fragments that included or lacked the RI region of NBD1 was also examined. In one experiment, a fragment (CFTR⁴³⁸) including the regulatory insert (“RI”, i.e., a 32-residue segment within the NBD1 domain) region and a fragment (CFTR³⁹²) lacking the RI region were evaluated in the presence of the active photoanalog having higher specific activity (FIG. 16A). In this experiment, the active photoanalog bound to the CFTR⁴³⁸ fragment and to the CFTR³⁹², indicating that the RI region is not required for binding of the active photoanalog to CFTR (FIG. 16B). Similar results were obtained in a separate experiment, in which a fragment including the RI region (CFTR⁴³⁸) and three fragments (CFTR³⁹², CFTR³⁸⁵, and CFTR³⁷⁶) lacking the regulatory insert (RI) region (i.e., a 32-residue segment within the NBD1 domain) were tested (FIG. 17A). In this experiment, the active photoanalog bound to the CFTR⁴³⁸ fragment and to the CFTR³⁹², indicating that the RI region is not required for binding of the active photoanalog to CFTR (FIG. 17B). Consistent with this data, lumacaftor was able to increase CFTR levels of ΔF508-CFTR mutants lacking the RI region (FIG. 17C).

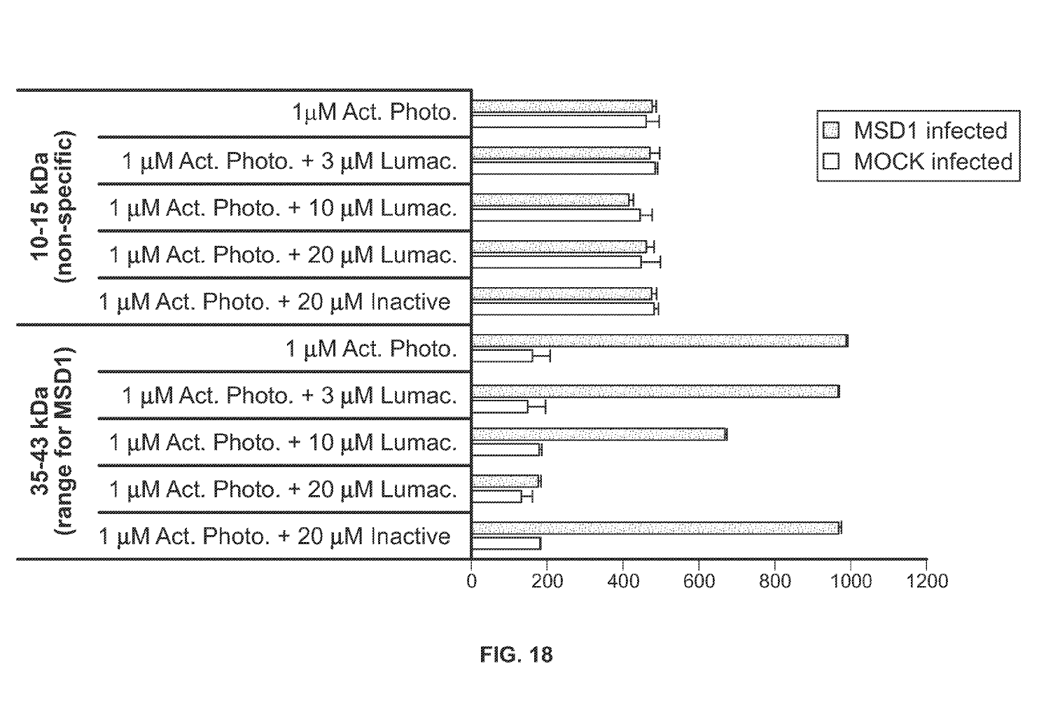

A dose-dependent competition experiments was also performed to assess whether lumacaftor and its active photoanalog interact with MSD1 differently. In this experiment, Sf9 cells that were mock-transfected or MSD1-transfected (generated as described above) were treated with the active photoanalog (having the low specific activity) and with increasing amounts of non-tritiated lumacaftor (3, 10 and 20 μM). Control cells were treated with the active photoanalog and with 20 μM of a non-tritiated, non-photoactive, inactive analog of lumacaftor (“non-tritiated inactive analog”). After labeling, Sf9 whole cell lysates were separated on a 4-12% Bis-Tris gel, and the areas of the gel corresponding to the 10-15 and 35-43 kDa range were cut and analyzed by a liquid scintillation counter. As illustrated in FIG. 18, binding of the active photoanalog to MSD1 from MSD1-transfected cells, but not the mock-transfected control cells, was reduced in the presence of lumacaftor in a concentration-dependent manner. The non-tritiated inactive analog had no effect on the binding between the active photoanalog and MSD1 from the MSD1-transfected cells. Increasing concentrations of non-tritiated lumacaftor and the non-tritiated inactive analog also had no effect on the non-specific binding of the active photoanalog to the polypeptide(s) in the 10-15 kDa gel fragment.

SEQUENCE INFORMATION

Human CFTR protein sequence (GenBank Accession No.
NP)000483.3)
SEQ ID NO: 1
MQRSPLEKASVVSKLFFSWTRPILRKGYRQRLELSDIYQIPSVDSADNL
SEKLEREWDRELASKKNPKLINALRRCFFWRFMFYGIFLYLGEVTKAVQ
PLLLGRIIASYDPDNKEERSIAIYLGIGLCLLFIVRTLLLHPAIFGLHH
IGMQMRIAMFSLIYKKTLKLSSRVLDKISIGQLVSLLSNNLNKFDEGLA
LAHFVWIAPLQVALLMGLIWELLQASAFCGLGFLIVLALFQAGLGRMMM
KYRDQRAGKISERLVITSEMIENIQSVKAYCWEEAMEKMIENLRQTELK
LTRKAAYVRYFNSSAFFFSGFFVVFLSVLPYALIKGIILRKIFTTISFC
IVLRMAVTRQFPWAVQTWYDSLGAINKIQDFLQKQEYKTLEYNLTTTEV
VMENVTAFWEEGFGELFEKAKQNNNNRKTSNGDDSLFFSNFSLLGTPVL
KDINFKIERGQLLAVAGSTGAGKTSLLMVIMGELEPSEGKIKHSGRISF
CSQFSWIMPGTIKENIIFGVSYDEYRYRSVIKACQLEEDISKFAEKDNI
VLGEGGITLSGGQRARISLARAVYKDADLYLLDSPFGYLDVLTEKEIFE
SCVCKLMANKTRILVTSKMEHLKKADKILILHEGSSYFYGTFSELQNLQ
PDFSSKLMGCDSFDQFSAERRNSILTETLHRFSLEGDAPVSWTETKKQS
FKQTGEFGEKRKNSILNPINSIRKFSIVQKTPLQMNGIEEDSDEPLERR
LSLVPDSEQGEAILPRISVISTGPTLQARRRQSVLNLMTHSVNQGQNIH
RKTTASTRKVSLAPQANLTELDIYSRRLSQETGLEISEEINEEDLKECF
FDDMESIPAVTTWNTYLRYITVHKSLIFVLIWCLVIFLAEVAASLVVLW
LLGNTPLQDKGNSTHSRNNSYAVIITSTSSYYVFYIYVGVADTLLAMGF
FRGLPLVHTLITVSKILHHKMLHSVLQAPMSTLNTLKAGGILNRFSKDI
AILDDLLPLTIFDFIQLLLIVIGAIAVVAVLQPYIFVATVPVIVAFIML
RAYFLQTSQQLKQLESEGRSPIFTHLVTSLKGLWTLRAFGRQPYFETLF
HKALNLHTANWFLYLSTLRWFQMRIEMIFVIFFIAVTFISILTTGEGEG
RVGIILTLAMNIMSTLQWAVNSSIDVDSLMRSVSRVFKFIDMPTEGKPT
KSTKPYKNGQLSKVMIIENSHVKKDDIWPSGGQMTVKDLTAKYTEGGNA
ILENISFSISPGQRVGLLGRTGSGKSTLLSAFLRLLNTEGEIQIDGVSW
DSITLQQWRKAFGVIPQKVFIFSGTFRKNLDPYEQWSDQEIWKVADEVG
LRSVIEQFPGKLDFVLVDGGCVLSHGHKQLMCLARSVLSKAKILLLDEP
SAHLDPVTYQIIRRTLKQAFADCTVILCEHRIEAMLECQQFLVIEENKV
RQYDSIQKLLNERSLFRQAISPSDRVKLFPHRNSSKCKSKPQIAALKEE
TEEEVQDTRL

We claim:

1. A method of treating cystic fibrosis in a patient, comprising the step of: administering to said patient a corrector agent capable of acting through the membrane spanning domain 1 (MSD1) during the biosynthesis of a CFTR protein, provided that the corrector agent is not a compound listed in Table 1, wherein said action is characterized in vitro by one or more of the following:
(i) an increase in accumulation of fragment CFTR³⁷⁵ in a cell expressing said fragment the presence of said corrector compared to such accumulation of fragment CFTR³⁷⁵ in a cell expressing said fragment in the absence of said corrector,
(ii) an increase in accumulation of fragment CFTR³⁸⁰ in a cell expressing said fragment in the presence of said corrector compared to such accumulation of fragment CFTR³⁸⁰ in a cell expressing said fragment in the absence of said corrector,
(iii) an increase in the half-life of fragment CFTR³⁷⁵ in a cell expressing said fragment in the presence of said corrector compared to such half-life of fragment CFTR³⁷⁵ in a cell expressing said fragment in the absence of said corrector,
(iv) an increase in the half-life of fragment CFTR³⁸⁰ in a cell expressing said fragment in the presence of said corrector compared to such half-life of fragment CFTR³⁸⁰ in a cell expressing said fragment in the absence of said corrector,
(v) an increase in the half-life of fragment CFTR³⁸⁰, CFTR⁴³⁰, and/or CFTR⁶⁵³ in a cell expressing CFTR³⁸⁰, CFTR⁴³⁰, and/or CFTR⁶⁵³ in the presence of said corrector compared to the half-life of CFTR³⁸⁰, CFTR⁴³⁰, and/or CFTR⁶⁵³, respectively, in a cell expressing said fragment in the absence of said corrector, or, or
(vi) an enhanced resistance of fragment CFTR³⁸⁰ to proteolysis with trypsin in the presence of said corrector compared to such proteolysis in the absence of said corrector.

2. The method of claim 1, wherein said corrector agent is capable of acting through the membrane spanning domain 1 (MSD1) during the biosynthesis of a mutant CFTR protein.

3. The method of claim 1 or 2, wherein the concentration of said corrector agent needed to achieve the maximal accumulation of fragment CFTR³⁸⁰ in a cell expressing said fragment is about the same concentration of said corrector agent needed to achieve the maximal accumulation of full-length CFTR in a cell expressing said full-length CFTR.

4. The method of claim 1 or 2, wherein said corrector agent action is characterized by one characteristic selected from characteristics (i)-(vi).

5. The method of claim 1 or 2, wherein said corrector agent action is characterized by two characteristics selected from characteristics (i)-(vi).

6. The method of claim 1 or 2, wherein said corrector agent action is characterized by three characteristics selected from characteristics (i)-(vi).

7. The method of claim 1 or 2, wherein said corrector agent action is characterized by four characteristics selected from characteristics (i)-(vi).

8. The method of claim 1 or 2, wherein said corrector agent action is characterized by five characteristics selected from characteristics (i)-(vi).

9. The method of claim 1 or 2, wherein said corrector agent action is characterized by six characteristics selected from characteristics (i)-(vii).

10. The method of claim 1 or 2, wherein said action of said corrector agent is characterized in vitro by: the concentration of said corrector agent needed to achieve the maximal accumulation of fragment CFTR³⁸⁰ in a cell expressing said fragment is about the same concentration of said corrector agent needed to achieve the maximal accumulation of full-length CFTR in a cell expressing said full-length CFTR.

11. The method of any one of claims 1-10, wherein said corrector acts through at least one amino acid residue selected from an amino acid residue corresponding to amino acid residues 362-380 of CFTR (SEQ ID NO: 1).

12. The method of claim 11, wherein said corrector acts through at least one amino acid residue selected from an amino acid residue corresponding to amino acid residues 371-375 of CFTR (SEQ ID NO: 1).

13. The method of any one of claims 1-12, wherein said increase in accumulation of fragment CFTR³⁷⁵ or fragment CFTR³⁸⁰ is an at least 2-fold increase in accumulation.

14. The method of claim 13, wherein said increase in accumulation of fragment CFTR³⁷⁵ or fragment CFTR³⁸⁰ is an at least 4-fold increase in accumulation.

15. The method of claim 13, wherein said increase in accumulation of fragment CFTR³⁷⁵ or fragment CFTR³⁸⁰ is an at least 6-fold increase in accumulation.

16. The method of any one of claims 1-12, wherein said increase in half-life of fragment CFTR³⁷⁵ or fragment CFTR³⁸⁰ is an at least 2-fold increase in half-life.

17. The method of claim 16, wherein said increase in half-life of fragment CFTR³⁷⁵ or fragment CFTR³⁸⁰ is an at least 4-fold increase in half-life.

18. The method of claim 17, wherein said increase in half-life of fragment CFTR³⁷⁵ or fragment CFTR³⁸⁰ is an at least 6-fold increase in half-life.

19. The method of any one of claims 1-18, wherein said corrector agent action is further characterized in vitro by an ability to increase chloride transport in the presence of said corrector in one or more of the following CFTR mutations: E56K, P67L, E92K, L206W and/or ΔF508

20. The method of any one of claims 1-19, wherein said corrector agent action is further characterized in vitro by a similar increase in accumulation of fragment CFTR³⁷⁰ or half-life of fragment CFTR³⁷⁰ in the presence of said corrector compared to such accumulation of fragment CFTR³⁷⁰ or half-life of fragment CFTR³⁷⁰, respectively, in the absence of said corrector.

21. The method of any one of claims 1-20, wherein said corrector agent does not increase accumulation of a fragment CFTR³⁸⁰ containing a mutation or deletion between residues 362-380.

22. The method of any one of claims 1-12, wherein said proteolysis of fragment CFTR³⁸⁰ by trypsin in the presence of said corrector produces an increased amount of a 22 kD protease resistant fragment.

23. The method of any one of claim 1-12, wherein said corrector agent is capable of increasing the amount of a protease resistant 22 kD fragment produced by the proteolysis of ΔF508 CFTR in the presence of said corrector.

24. The method of any one of claims 1-23, wherein said corrector agent is capable of promoting interaction between MSD1 and nuclear binding domain 1 (NBD1) in a CFTR protein.

25. The method of claim 24, wherein the interaction between MSD1 and NBD1 is between intracellular loop 1 (ICL1) and NBD1.

26. The method of any one of claims 1-25, wherein said corrector agent is capable of selectively interacting with CFTR protein.

27. The method of claim 26, wherein said corrector agent is not capable of interacting with any of an ion channel other than CFTR, an ABC transporter other than CFTR, a misfolded protein other than mutant CFTR, a G-protein coupled receptor, a kinase, a molecular chaperone, an ER stress marker and activation marker.

28. The method of any one of claims 1-27, wherein said corrector agent is capable of interacting with MSD1 of CFTR prior to the synthesis of NBD1.

29. The method of any one of claims 2-28, wherein said mutant CFTR protein in the presence of the corrector agent in vitro is less susceptible to ER associated degradation (ERAD) than is the mutant CFTR protein in the absence of the corrector agent in vitro.

30. The method of any one of claims 2-29, wherein said mutant CFTR protein in the presence of the corrector agent in vitro is less susceptible to degradation by a proteasome than is the mutant CFTR protein in the absence of the corrector agent in vitro.

31. The method of any one of claims 1-30, wherein the susceptibility to ER associated degradation (ERAD) of said mutant CFTR protein in the presence of the corrector agent in vitro is more similar to the susceptibility to ERAD of a wildtype CFTR than to the susceptibility to ERAD of the mutant CFTR protein in the absence of the corrector agent in vitro.

32. The method of any one of claims 1-31, wherein the susceptibility to degradation by a proteasome of said mutant CFTR protein in the presence of the corrector agent in vitro is more similar to the susceptibility to degradation by a proteasome of a wildtype CFTR protein than to the susceptibility to degradation by a proteasome of the mutant CFTR protein in the absence of the corrector agent in vitro.

33. The method of any one of claims 2-32, wherein said mutant CFTR protein in the presence of the corrector agent in vitro is at least 100% more resistant to proteolysis than the mutant CFTR protein in the absence of the corrector agent in vitro.

34. The method of claim 31, wherein said mutant CFTR protein in the presence of the corrector agent in vitro is at least 200% more resistant to proteolysis than the mutant CFTR protein in the absence of the corrector agent in vitro.

35. The method of claim 32, wherein said mutant CFTR protein in the presence of the corrector agent in vitro is at least 250% more resistant to proteolysis than the mutant CFTR protein in the absence of the corrector agent in vitro.

36. The method of any one of claims 33-35, wherein said proteolysis resistance is proteolysis resistance of NBD2 in said mutant CFTR protein.

37. The method of any one of claims 33-35, wherein the proteolysis resistance is trypsin resistance.

38. The method of any one of claims 33-35, wherein the proteolysis resistance is V8 protease resistance.

39. The method of any one of claims 1-38, wherein said accumulation of said NBD1 fragment, ΔF508-NBD1 fragment, fragment CFTR 375 and/or fragment CFTR 380 is determined by Western Blot.

40. The method of any one of claims 1-39, wherein said corrector agent does not bind MSD2.

41. The method of any one of claims 1-40, wherein said CFTR protein is capable of being potentiated by ivacaftor.

42. The method of any one of claims 1-41, wherein said method further comprises the step of administering to said patient one or more additional therapeutic agents, wherein said additional therapeutic agent is a CFTR potentiator.

43. The method of claim 42, wherein said CFTR potentiator is ivacaftor or a pharmaceutically acceptable salt thereof.

44. The method of any one of claims 1-43, wherein said method further comprises the step of administering to said patient one or more additional therapeutic agents, wherein said additional therapeutic agent is selected from the group consisting of a bronchodilator, an antibiotic, a mucolytic agent, a nutritional agent and an agent that blocks ubiquitin-mediated proteolysis.

45. The method of claim 44, wherein said additional therapeutic agent is an agent that blocks ubiquitin-mediated proteolysis.

46. The method of claim 45, wherein said agent that blocks ubiquitin-mediated proteolysis is a proteasome inhibitor.

47. The method of claim 46, wherein said agent that blocks ubiquitin-mediated proteolysis is selected from the group consisting of a peptide aldehyde, a peptide boronate, a peptide α′β′-epoxyketone, a peptide ketoaldehyde or a β-lactone.

48. The method of claim 47, wherein said agent that blocks ubiquitin-mediated proteolysis is selected from the group consisting of bortezomib, carfilzomib, marizomib, CEP-18770, MLN-9708 and ONX-0912.

49. The method of any one of claims 1-48, wherein said patient has a mutant CFTR protein and wherein said mutant CFTR protein comprises a mutation in the MSD1 domain of the CFTR protein.

50. The method of claim 49, wherein said mutant CFTR protein comprises a mutation in the transmembrane 1 (TM1).

51. The method of claim 50, wherein said mutant CFTR protein comprises a mutation at an amino acid position corresponding to amino acid residue 92 of SEQ ID NO: 1.

52. The method of claim 51, wherein said mutant CFTR protein comprises a mutation selected from the group consisting of a substitution of lysine, glutamine, arginine, valine or aspartic acid for glutamic acid at amino acid residue 92 of SEQ ID NO: 1.

53. The method of claim 49, wherein said mutant CFTR protein comprises a mutation in the transmembrane 2 (TM2) region.

54. The method of claim 53, wherein said mutant CFTR protein comprises a mutation at an amino acid position corresponding to amino acid residue 139 of SEQ ID NO: 1.

55. The method of claim 54, wherein said mutant CFTR protein comprises a substitution of arginine for histidine at amino acid residue 139 of SEQ ID NO: 1.

56. The method of claim 49, wherein said mutant CFTR protein comprises mutation is in the transmembrane 3 (TM3) region.

57. The method of claim 56, wherein said mutant CFTR protein comprises a mutation at the amino acid position corresponding to amino acid residue 206 of SEQ ID NO: 1.

58. The method of claim 57, wherein said mutant CFTR protein comprises a substitution of leucine for tryptophan at amino acid residue 206 of SEQ ID NO:1.

59. The method of claim 49, wherein said mutant CFTR protein comprises a mutation in the transmembrane 4 (TM4) region.

60. The method of claim 49, wherein said mutant CFTR protein comprises a mutation in the transmembrane 5 (TM5) region of the CFTR protein.

61. The method of claim 49, wherein said mutant CFTR protein comprises a mutation in the transmembrane 6 (TM6) region of the CFTR protein.

62. The method of any one of claims 1-61, wherein said patient has a mutant CFTR protein and wherein said mutant CFTR protein comprises a mutation in a coupling helix extending from transmembrane 2 (TM2) region or transmembrane 3 (TM3) region of the CFTR protein.

63. The method of claim 62, wherein said mutant CFTR protein comprises a mutation at an amino acid position corresponding to amino acid residue 149 or 192 of SEQ ID NO: 1.

64. The method of any one of claims 1-63, wherein said patient has a mutant CFTR protein and wherein said mutant CFTR protein comprises a mutation in the nuclear binding domain 1 (NBD1) domain of CFTR protein.

65. The method of claim 64, wherein said mutant CFTR protein comprises a deletion of phenylalanine at amino acid residue 508 of SEQ ID NO: 1.

66. The method of claim 28, wherein said corrector agent is capable of promoting interaction between ICL4 and NBD1 in the CFTR protein.

67. The method of claim 28 or 66, wherein said corrector agent is capable promoting said interaction in vitro.

68. The method of any one of claims 1-67, wherein said corrector agent is a non-naturally occurring agent.

69. The method of claim 68, wherein said corrector agent is a non-naturally occurring polypeptide corrector agent.

70. The method of claim 68, wherein said corrector agent is a non-naturally occurring antibody or antibody fragment.

71. The method of claim 68, wherein said corrector agent is a small molecule.

72. The method of any one of claims 1-71, wherein said corrector is formulated with a pharmaceutically acceptable carrier.

73. The method of any one of claims 1-72, wherein said corrector agent is administered to said patient orally, sublingually, intravenously, intranasally, subcutaneously or intra-muscularly.

74. The method of any one of claims 1-73, wherein said corrector agent is orally administered to said patient.

75. The method of claim 43, wherein said corrector agent and ivacaftor are orally administered to said patient.

76. The method of any one of claims 42-48, wherein said corrector agent and said one or more additional therapeutic agents are concurrently administered to said patient.

77. The method of any one of claims 42-48, wherein said corrector agent and said one or more additional therapeutic agents are administered consecutively to said patient.

78. The method of any one of claims 42-48, wherein said corrector agent and said one or more additional therapeutic agents are administered to said patient in a single formulation.

79. The method of any one of claims 42-48, wherein said corrector agent and said one or more additional therapeutic agents are administered to said patient in separate formulations.

80. A method of screening for a candidate corrector agent comprising the steps of:
a) contacting a test agent with a cell expressing a CFTR fragment, wherein the CFTR fragment is a fragment CFTR³⁷⁵ or a fragment CFTR³⁸⁰,
b) measuring the accumulation of the CFTR protein fragment in the cell, and
c) comparing the accumulation of the CFTR protein fragment in the cell with the accumulation of the CFTR protein fragment in a cell not contacted with the test agent,
wherein if the accumulation of CFTR protein fragment in the cell contacted with the test agent is greater than the accumulation of CFTR protein fragment in the cell not contacted with the test agent, the test agent is a candidate corrector agent.

81. A method of screening for a candidate corrector agent comprising the steps of:
a) contacting a test agent with a cell expressing a CFTR fragment, wherein the CFTR fragment is an NBD1 fragment, a ΔF508-NBD1 fragment or a CFTR³⁷⁰ fragment,
b) measuring the accumulation of the CFTR protein fragment in the cell, and
c) comparing the accumulation of the CFTR protein fragment in the cell with the accumulation of the CFTR protein fragment in a cell not contacted with the test agent,
wherein if the accumulation of CFTR protein fragment in the cell contacted with the test agent is greater than the accumulation of CFTR protein fragment in the cell not contacted with the test agent, the test agent is a candidate corrector agent.

82. The method of claim 80 or 81, wherein said accumulation of CFTR protein fragment is determined by Western Blot.

83. A method of screening for a candidate corrector agent comprising the steps of:
a) contacting a test agent with a cell expressing a CFTR protein,
b) measuring the amounts of mature CFTR protein in the cell,
c) comparing the amounts of mature CFTR protein in the cell with the amounts of the CFTR protein fragment in a cell not contacted with the test agent, and,
wherein if the amounts of mature CFTR protein in the cell contacted with the test agent is greater than the amounts of mature CFTR protein in the cell not contacted with the test agent, the test agent is a candidate corrector agent.

84. The method of claim 83, wherein the amounts of said mature CFTR protein is determined by Western Blot.

85. A method of screening for a candidate corrector agent comprising the steps of:
a) contacting a test agent with a cell expressing a mutant CFTR protein,
b) measuring the amounts or patterns of ubiquitination of the mutant CFTR protein in the cell, and
c) comparing the amounts or patterns of ubiquitination of the mutant CFTR protein in the cell with the ubiquitination patterns or amounts of the mutant CFTR protein in a cell not contacted with the test agent,
wherein if the amounts or patterns of ubiquitination of the mutant CFTR protein in the cell contacted with the test agent are different than the amounts or patterns of mutant CFTR protein in the cell not contacted with the test agent, the test agent is a candidate corrector agent.

86. A method of screening for a candidate corrector agent comprising the steps of:
a) contacting a test agent with a cell expressing a CFTR protein,
b) measuring the ER export of the CFTR protein in the cell, and
c) comparing the ER export of the CFTR protein in the cell contacted with the test agent with the ER export of the CFTR in a cell not contacted with the test agent,
wherein if the ER export of the CFTR protein in the cell contacted with the test agent is greater than the ER export of the CFTR protein in the cell not contacted with the test agent, the test agent is a candidate corrector agent.

87. The method of claim 86, wherein ER export is determined by a utilizing pulse-chase assay.

88. A method of screening for a candidate corrector agent comprising the steps of:
a) contacting a test agent with a cell expressing a CFTR protein,
b) measuring the chloride transport of the CFTR protein in the cell, and
c) comparing the chloride transport of the CFTR protein in the cell with the chloride transport of the CFTR protein in a cell not contacted with the test agent,
wherein if the chloride transport of the CFTR protein in the cell contacted with the test agent is greater than the chloride transport of the CFTR protein in the cell not contacted with the test agent, the test agent is a candidate corrector agent.

89. The method of claim 88, wherein said chloride transport is determined by measuring ion flow across cell membranes of cells expressing said CFTR protein.

90. The method of claim 89, wherein said measurement of ion flow is performed by utilizing Ussing chamber recording analysis.

91. A method of screening for a candidate corrector agent comprising the steps of:
a) contacting a test agent with a cell expressing a CFTR protein,
b) measuring the CFTR protein channel gating in the cell, and
c) comparing the CFTR protein channel gating in the cell with the CFTR protein channel gating in a cell not contacted with the test agent,
wherein if the channel gating of the CFTR protein in the cell contacted with the test agent is greater than the channel gating of the CFTR protein in the cell not contacted with the test agent, the test agent is a candidate corrector agent.

92. The method of claim 91, wherein the amount of channel gating is determined by single-channel patch clamp recording analysis.

93. A method of screening for a candidate corrector agent comprising the steps of:
a) contacting a test agent with a cell expressing a CFTR protein,
b) measuring the ATPase activity of the CFTR protein in the cell, and
c) comparing the ATPase activity of the CFTR protein in the cell with the ATPase activity of the CFTR protein in a cell not contacted with the test agent,
wherein if the ATPase activity of the CFTR protein in the cell contacted with the test agent is greater than the ATPase activity of the CFTR protein in the cell not contacted with the test agent, the test agent is a candidate corrector agent.

94. The method of any one of claims 80-93, wherein the candidate corrector agent is a corrector agent.

95. A pharmaceutical composition comprising:
a) a corrector agent as defined in any one of claims 1-79, and
b) a pharmaceutically acceptable acceptable carrier, adjuvant or vehicle.
